(19) United States
(12) Patent Application Publication
(10) Pub. No.: US 2026/0272944 A1
(43) Pub. Date: Sep. 17, 2026

(54) PYRIDO[3,4-D]PYRIMIDINONE AND PYRIMDLLNE ARYL HYDROCARBON RECEPTOR ANTAGONISTS AND USES THEREOF

(71) Applicants: ALLIANTHERA (SUZHOU) BIOPHARMACEUTICAL CO., LTD., Suzhou, Jiangsu (CN); ALLIANTHERA BOSTON INC., Natick Massachusetts, MA (US)

(72) Inventors: Jiamin GU, Suzhou (CN); Lei ZHANG, Suzhou (CN); Chengzhe WU, Suzhou (CN); Xianchao DU, Suzhou (CN); Meng DU, Suzhou (CN); Jinshan CHEN, Natick Massachusetts, MA (US)

(21) Appl. No.: 19/471,448

(22) PCT Filed: Apr. 12, 2024
(86) PCT No.: PCT/US2024/024177

§ 371 Date: Oct. 1, 2025

(30) Foreign Application Priority Data
Apr. 13, 2023 (WO) PCT/CN2023/087979

(51) Int. Cl. A61K 31/5386; A61K 31/506; A61K 31/519; A61K 31/5377; A61K 31/551; A61K 45/06; C07D 239/42; C07D 401/14; C07D 403/04; C07D 403/14; C07D 405/14; C07D 413/14; C07D 471/04; C07D 498/08; C07D 519/00

(52) U.S. Cl. CPC A61K 31/5386; A61K 31/506; A61K 31/519; A61K 31/5377; A61K 31/551; A61K 45/06; C07D 239/42; C07D 401/14; C07D 403/04; C07D 403/14; C07D 405/14; C07D 413/14; C07D 471/04; C07D 498/08; C07D 519/00

(57) ABSTRACT

Disclosed herein are compounds which can act as modulators of the aryl hydrocarbon receptor (AHR). Further disclosed herein are methods for treating diseases and disorders, such as cancer and viral infections, using the compounds disclosed herein.

### FIELD

This disclosure relates to compounds that are useful as aryl hydrocarbon receptor (AHR) activity antagonists, pharmaceutical formulations thereof, and methods of using the compounds to treat diseases and disorders, such as cancer.

### BACKGROUND

The aryl hydrocarbon receptor (AHR) is a ligand-dependent transcription factor that regulates gene expression in a variety of cells, such as epithelial and immune cells. Emerging evidence suggests that AHR plays a role in the initiation, promotion, progression, invasion, and metastasis of cancer cells. Various tumor types and tumor cell lines show high AHR expression, suggesting that AHR is activated in tumors and facilitates their growth. Immune evasion is gaining recognition as a hallmark feature of cancer. A connection between the AHR and immune system has been identified, and AHR has been suggested as an immunosuppressive effector on different types of immune cells. Certain cancers can escape immune recognition via AHR signaling pathways. Overall, modulating AHR activity in cancer cells, immune cells, stromal cells, fibroblasts, and endothelial cells within a tumor can slowdown the progression of the disease.

AHR is expressed at high levels and is chronically active in blood tumors, such as T-cell leukemia and lymphoma, as well as in solid tumors, such as glioblastoma, ovarian cancer, lung cancer, liver cancer, and head and neck carcinomas. It has been suggested that detection of AHR activity in the tumor microenvironment can serve as a potent diagnostic indicator for tumor aggressiveness. Depending on the cancer type, two types of results are associated with AHR activity and the prognosis. There is evidence that, in hormone-dependent breast cancers, AHR activation is associated with attenuated aggressiveness and a better prognosis. In contrast, higher AHR activity has been suggested as being correlated with increased aggressiveness and a poor prognosis in non-small-cell lung cancer.

Recent reports have also demonstrated the role of AHR as a modulator of the intrinsic, innate and adaptive immune response to viral infections. The role of AHR activation has been recognized as influencing host resistance to infections by a variety of viruses, including influenza, coronaviruses (such as SARS-CoV-1, SARS-CoV-2, and MERS-CoV), flaviviruses (such as Zika virus), retroviruses (such as HIV), and herpesviruses.

As such, there is a need for novel compounds that act as antagonists to AHR to provide new and effective therapies for diseases and disorders, such as cancer and viral infections.

### SUMMARY

Provided herein are compounds of Formula (I) or Formula (II):

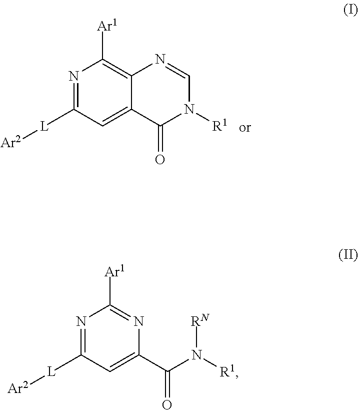

wherein substituents are as discussed below. These compounds are useful as aryl hydrocarbon receptor modulators.

### DETAILED DESCRIPTION

Provided herein are compounds of Formula (I):

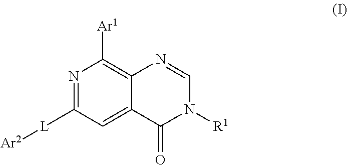

### or Formula (II):

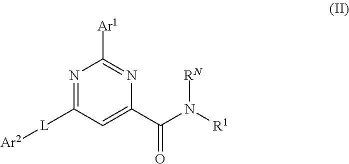

wherein L, R¹, R^{N}, Ar¹, and Ar² are as described herein, which can function as modulators of AHR activity. For example, compounds as disclosed herein can increase or decrease AHR activity.

### Compounds of the Disclosure

Provided herein are compounds, or pharmaceutically acceptable salts thereof, having a structure of Formula (I) or Formula (II):

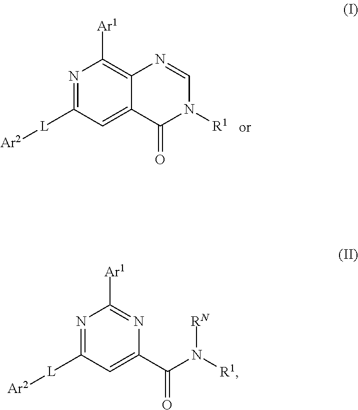

wherein
L is a bond, C(O), C₂₋₃alkenylene, or C₂₋₃alkynylene;
Ar¹ is phenyl, C₃₋₈cycloalkyl, or 4-8-membered aromatic or non-aromatic heterocyclyl having 1-3 ring heteroatoms selected from O, N, and S, and Ar¹ is optionally substituted with 1 or R^{A}; 2
each R^{A} is independently halo, OH, ═O, CN, C₁₋₆alkyl, C₁₋₆haloalkyl, C₁₋₆hydroxyalkyl, C₁₋₆alkoxy, C₁₋₆haloalkoxy, cyclopropyl, NR^{N}R^{N}, C(O)NR^{N}R^{N}, CO₂R^{N}, C(O)R^{N}, or SO₂—C₁₋₆alkyl;
each R^{N} is independently H or C₁₋₆alkyl;
Ar² is 5-10 membered aromatic or non-aromatic ring having 0-3 ring heteroatoms selected from O, N, and S, and Ar² is optionally substituted with 1, 2, or 3 R^{B};
each R^{B} is independently halo, CN, C₁₋₆alkyl, OH, O, C₁₋₆hydroxyalkyl, C(O)R^{N}, CO₂R^{N}, C(O)NR^{N}R^{N}, Het, C(O)—C₀₋₂alkylene-Het, or N(R^{N})—C₀₋₃alkylene-C(O)—C₀₋₃alkylene-Het;
Het is a 4-8-membered aromatic or non-aromatic ring having 0-3 ring heteroatoms selected from N, O, and S, and is optionally substituted with 1, 2, or 3 R^{C};
each R^{C} is independently halo, OH, ═O, CN, C₁₋₆alkyl, C₁₋₆haloalkyl, C₁₋₆hydroxyalkyl, C₁₋₆alkoxy, C₁₋₆haloalkoxy, NR^{N}R^{N}, C(O)NR^{N}R^{N}, CO₂R^{N}, C(O)R^{N}, or SO₂—C₁₋₆alkyl;
R¹ is C₁₋₆alkyl, C₁₋₆alkylene-R^{D}, C₁₋₆alkylene-C(O)R^{D}, C₁₋₆alkylene-NHSO₂cyclopropyl, or C₁₋₆alkylene-NR^{N}—C(O)R^{N}; and
R^{D} is OR^{N} or NR^{N}R^{N}, with the proviso that
(i) for compounds of Formula (I), when L is a bond, then (a) Ar² is substituted with at least one R^{B} selected from Het, C(O)—C₀₋₂alkylene-Het, and N(R^{N})—C₀₋₃alkylene-C(O)—C₀₋₃alkylene-Het or (b) Ar² comprises a 8-10-membered bicyclic aromatic or non-aromatic ring having 1-3 ring heteroatoms and is substituted with 0-3 R^{B}; and
(ii) for compounds of Formula (II), when L is a bond, Ar² is phenyl or a 6-membered aromatic heterocyclyl, Ar² is substituted with 1 R^{B}, and R^{B} is Het, then (a) Het is attached to Ar² via a carbon ring atom of Het or (b) Het is substituted with 1, 2, or 3 R^{C}.

In some cases, each R^{N} independently is H or CH₃. In some cases, R^{N} is H. In some cases, R^{N} is C₁₋₆alkyl. In some cases, R^{N} is methyl.

In some cases, R¹ is C₁₋₆alkyl or C₁₋₆alkylene-R^{D}. In some cases, R^{D} is OR^{N}. In some cases, R^{D} is OH. In some cases, R¹ is R¹ is

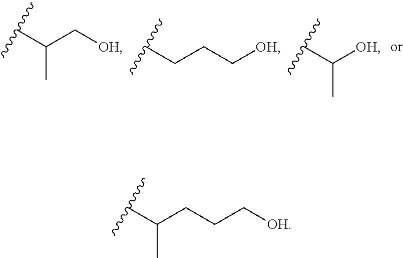

In some cases, R¹ is

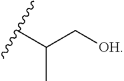 [SMILES: *C(C)CO]

In some cases, R¹ is

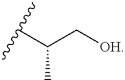 [SMILES: *[C@@H](C)CO]

In some cases, the compound has a structure of Formula (Ia) or (IIa):

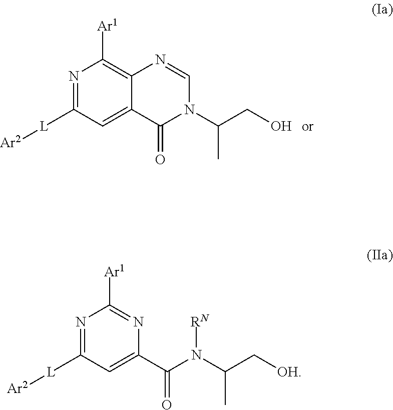

In various cases, Ar¹ can be heterocyclyl comprising 4-8 total ring atoms, wherein 1-3 of the ring atoms are selected from O, N, and S. In various cases, Ar¹ is phenyl, C₃₋₈cycloalkyl, or pyrazole. In some cases, Ar¹ is unsubstituted. In some cases, the Ar¹ is substituted with 1 or 2 R^{A}, e.g., at least one R^{A} is C₁₋₆alkyl, C₁₋₆haloalkyl, or C₁₋₆alkoxy. In some cases, at least one R^{A} is methyl.

In some cases, the compound has a structure of Formula (Ib) or (IIb):

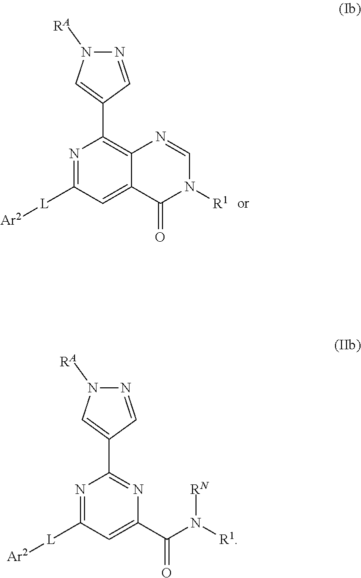

In some cases, the compound has a structure of Formula (Ic) or (IIc):

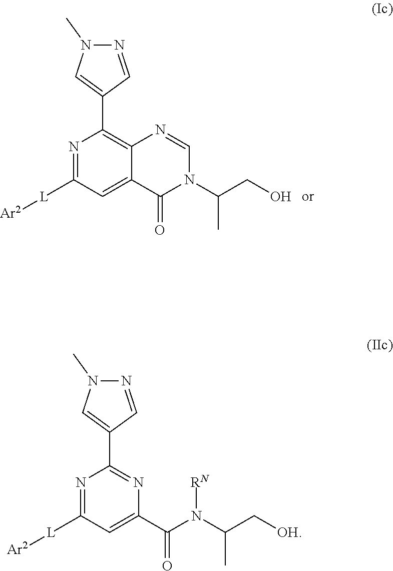

In various cases, L is a bond. In some cases, L is C₂₋₃alkenylene. In some cases, L is C₂₋₃alkynylene.

In various embodiments, Ar² is phenyl or naphthyl. In various embodiments, Ar² is a 5-10 membered aromatic or non-aromatic ring having 1-3 ring heteroatoms selected from O, N, and S. In some cases, Ar² is isoindoline, tetrahydroisoquinoline, tetrahydroquinoline, indazole, or pyrazolo[4,3-c]pyridine. In various embodiments, Ar² is unsubstituted. In some cases, Ar² is substituted with 1, 2, or 3 R^{B}. In some cases, at least one R^{B} is halo, CN, C₁₋₆alkyl, OH, ═O, C₁₋₆hydroxyalkyl, C(O)R^{N}, CO₂R^{N}, or C(O)NR^{N}R^{N}. In various cases, at least one R^{B} is Het, C(O)—C₀₋₂alkylene-Het, or N(R^{N})—C₀₋₃alkylene-C(O)—C₀₋₃alkylene-Het. In some cases, Het comprises phenyl, pyridine, pyridine-2-one, pyrimidine, piperidine, piperazine, morpholine, azepane, 1,4-diazepane, pyrrolidine, tetrahydropyran, dihydropyran, or 8-oxa-3λ²-azabicyclo[3.2.1]octane. In some cases, Het is unsubstituted. In various cases, Het is substituted with 1 or 2 R^{C}. In some cases, at least one R^{C} is halo, OH, ═O, C₁₋₆alkyl, CO₂R^{N}, NR^{N}R^{N}, or SO₂—C₁₋₆alkyl. In some cases, Het is

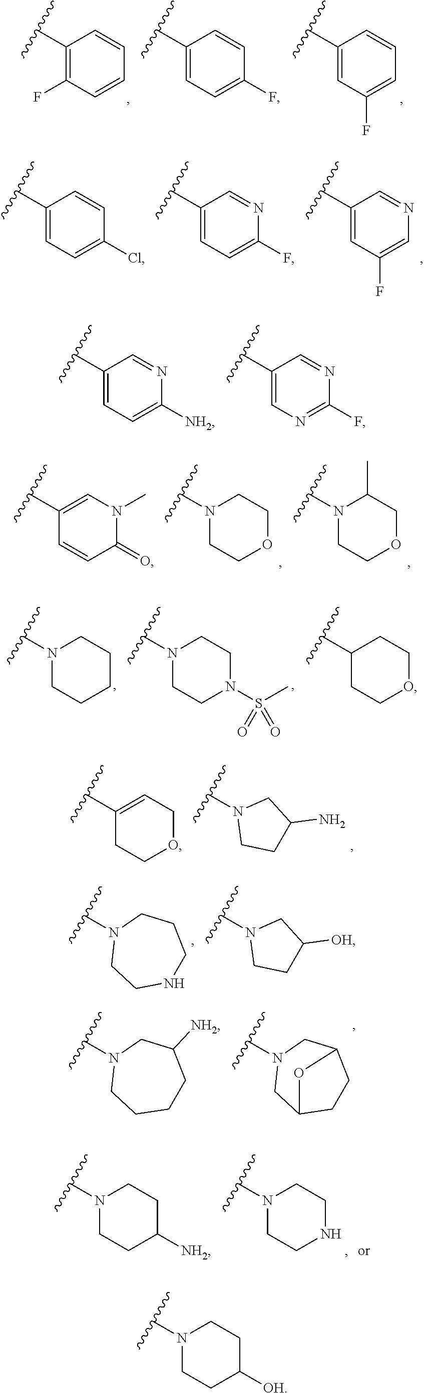

In various cases for compounds of Formula (I), Ar² is

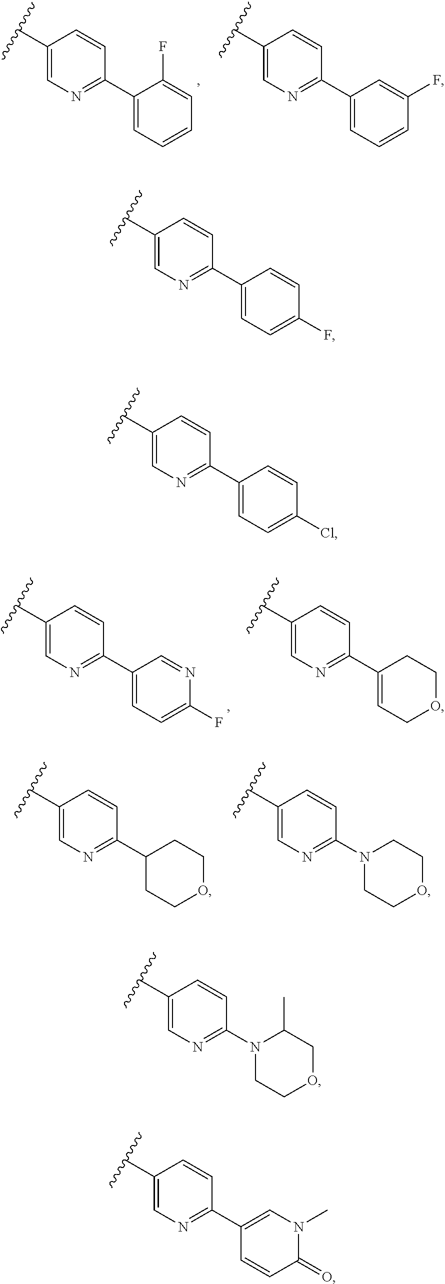

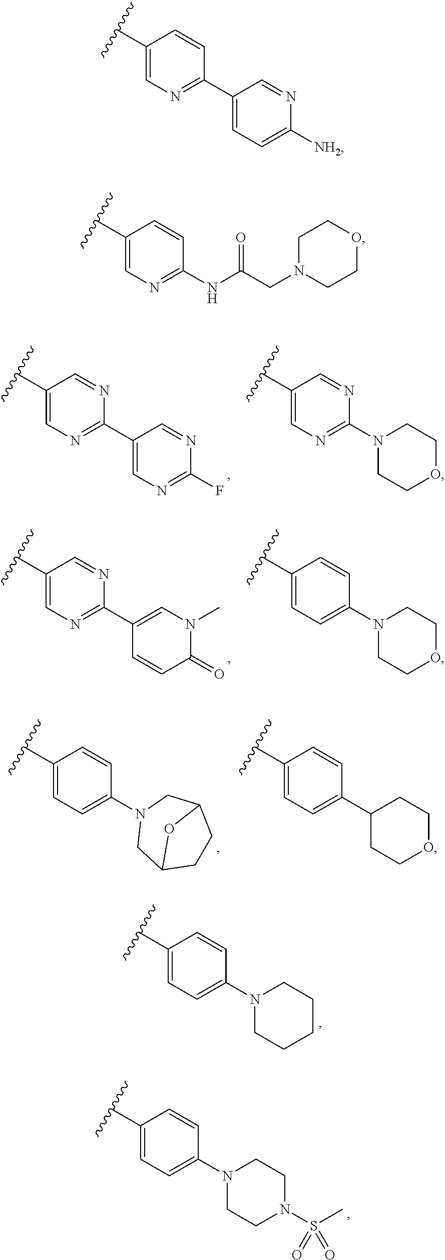

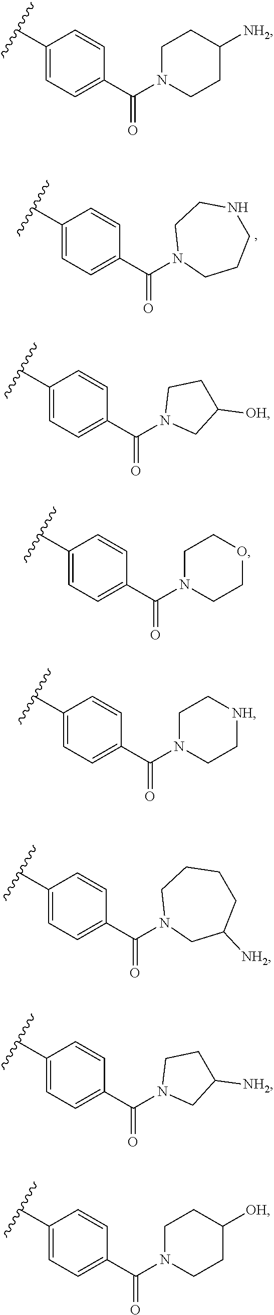

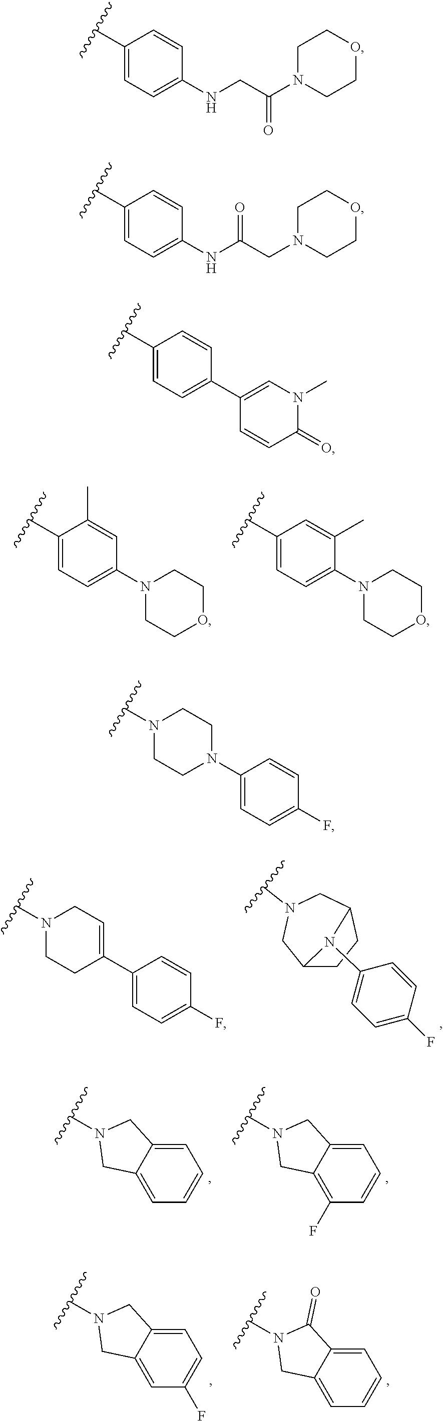

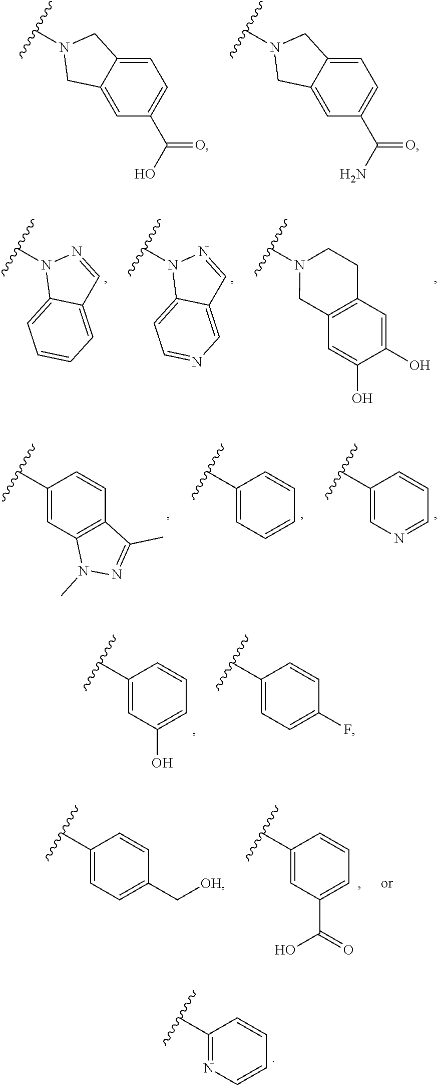

In various cases for compounds of Formula (II), Ar² is

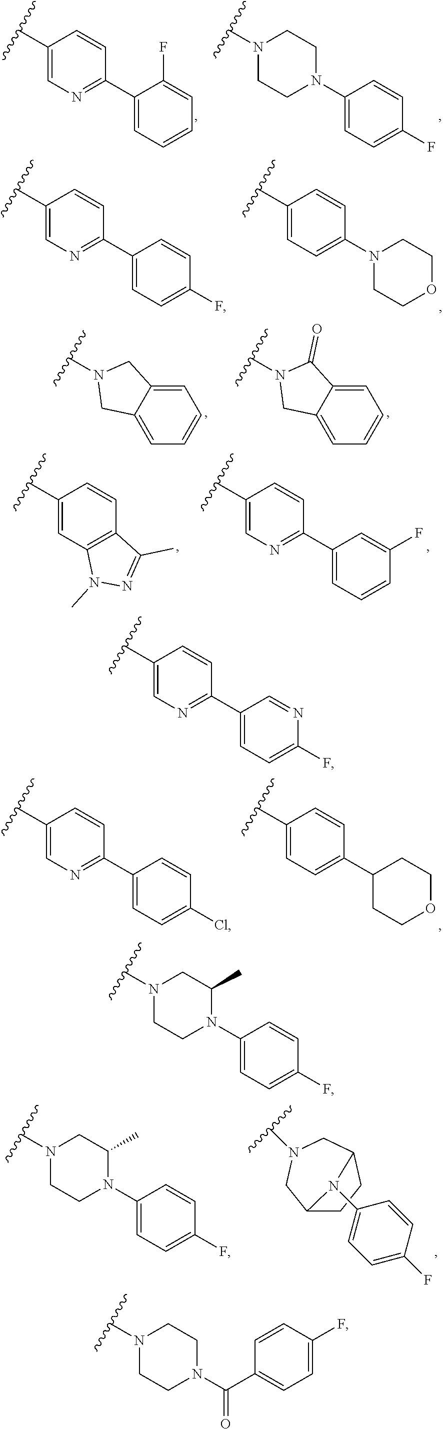

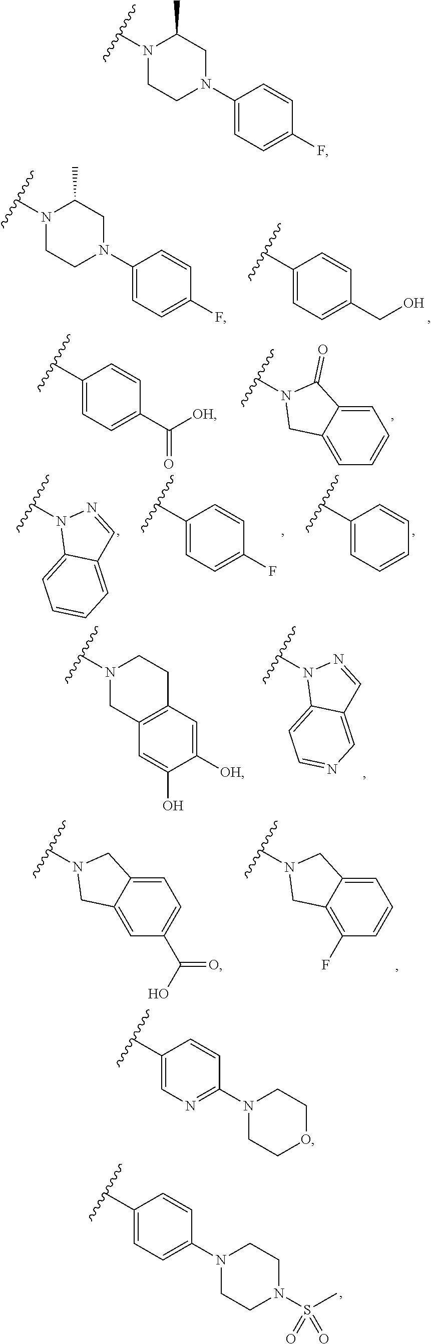

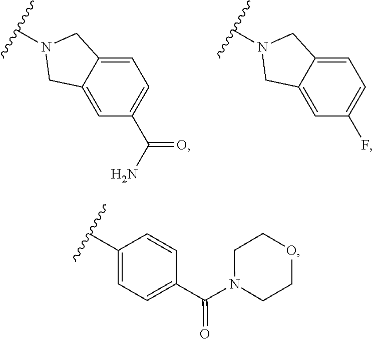 [SMILES: *N1Cc2ccc(C(N)=O)cc2C1 ; *N1Cc2ccc(F)cc2C1 ; *c1ccc(C(=O)N2CCOCC2)cc1]

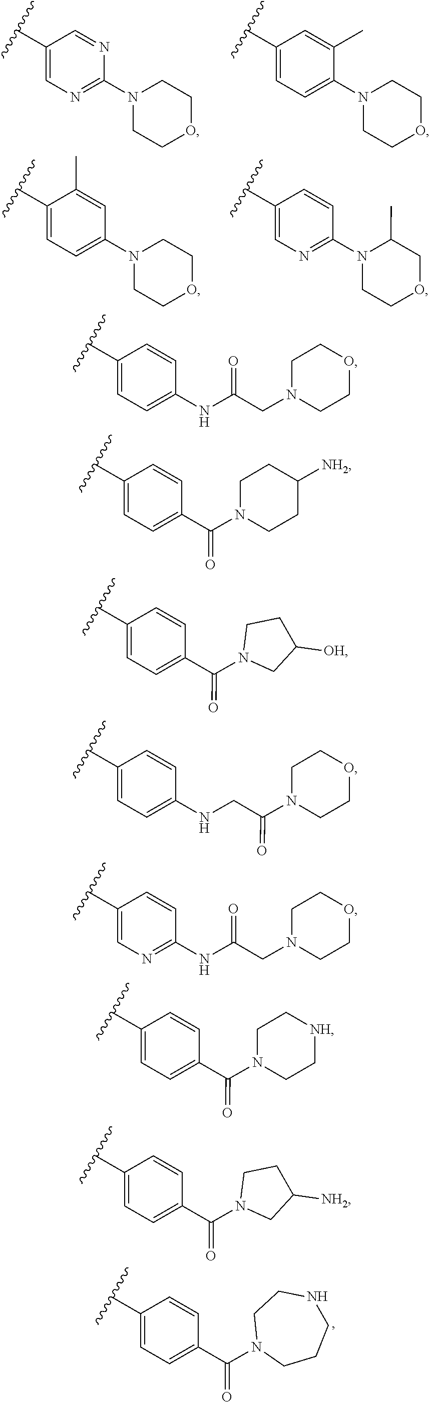

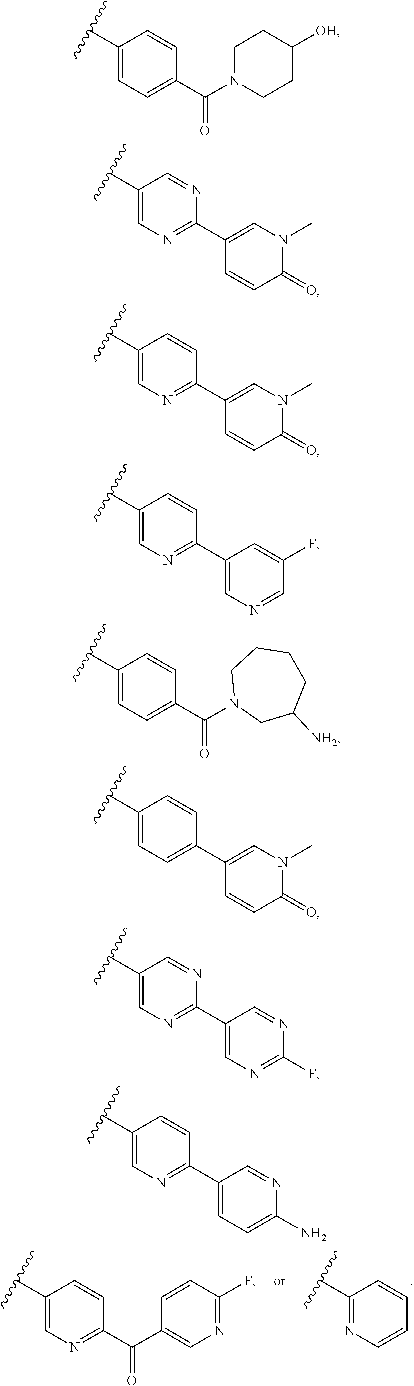

Specific compounds disclosed herein include those listed in Table 1, or a pharmaceutically acceptable salt thereof.

TABLE 1

| # | Structure |
|---|---|
| 1 | 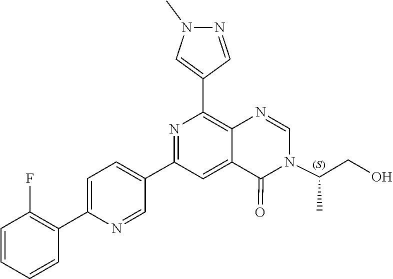 |
| 2 | 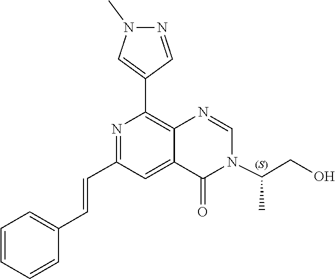 |
| 3 | 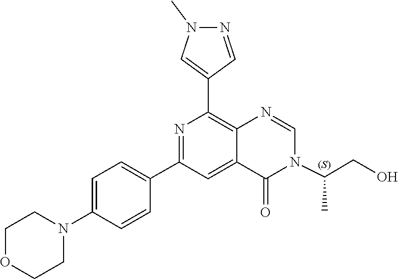 |
| 4 | 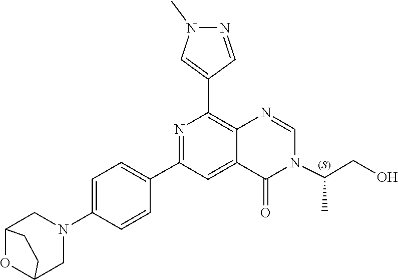 |
| 5 | 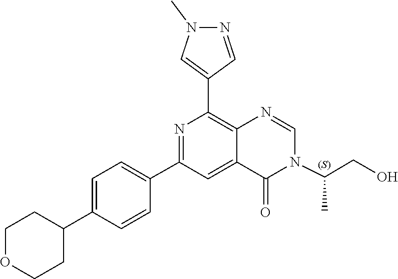 |
| 6 | 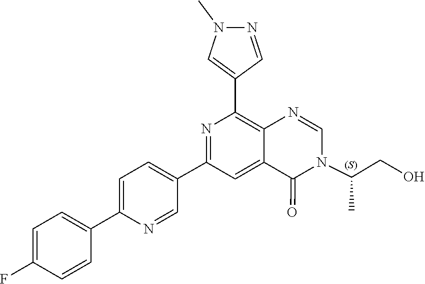 |
| 7 | 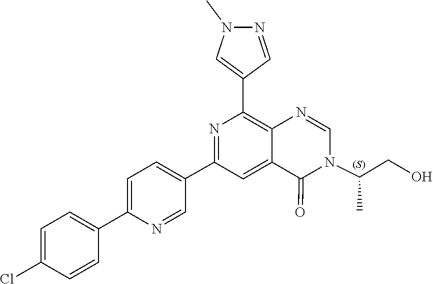 |
| 8 | 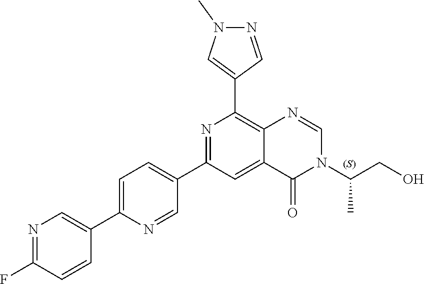 |
| 9 | 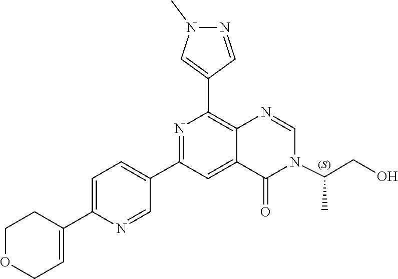 |
| 10 | 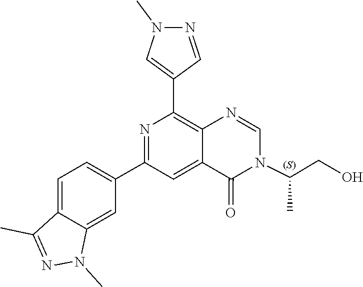 |
| 11 | 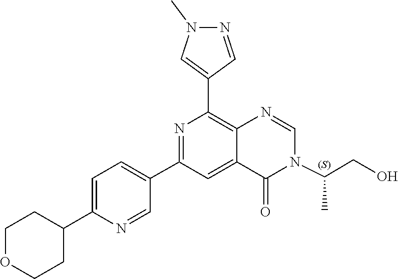 |
| 12 | 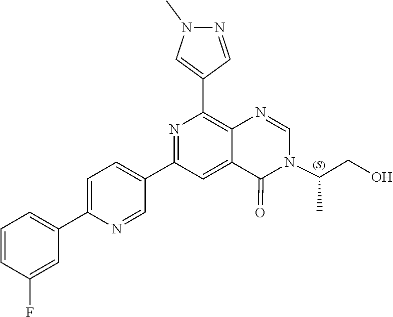 |
| 13 | 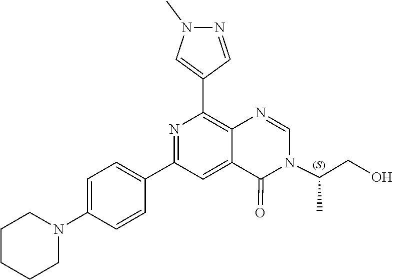 |
| 14 | 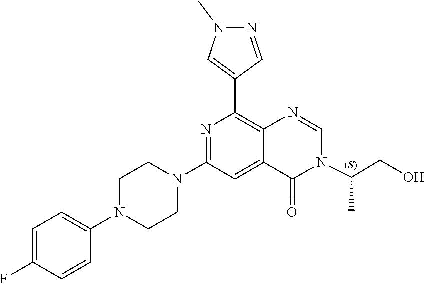 |
| 15 | 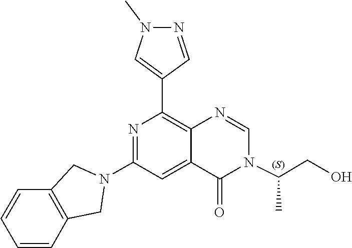 |
| 16 | 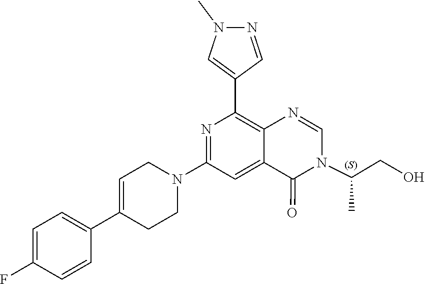 |
| 17 | 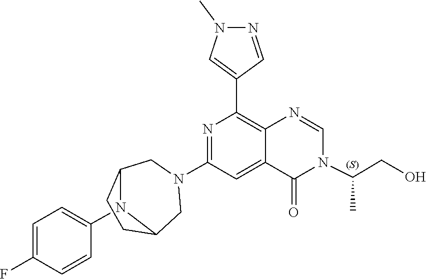 |
| 18 | 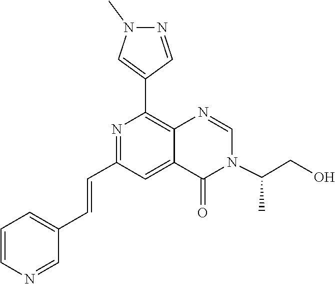 [SMILES: C[C@@H](CO)n1cnc2c(-c3cnn(C)c3)nc(/C=C/c3cccnc3)cc2c1=O] |
| 19 | 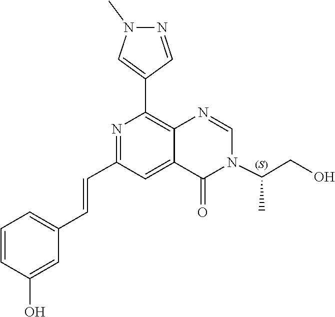 |
| 20 | 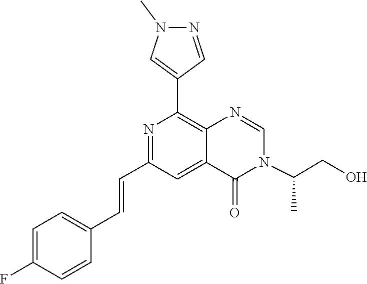 [SMILES: C[C@@H](CO)n1cnc2c(-c3cnn(C)c3)nc(/C=C/c3ccc(F)cc3)cc2c1=O] |
| 21 | 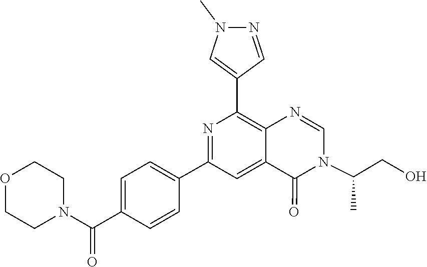 [SMILES: C[C@@H](CO)n1cnc2c(-c3cnn(C)c3)nc(-c3ccc(C(=O)N4CCOCC4)cc3)cc2c1=O] |
| 22 | 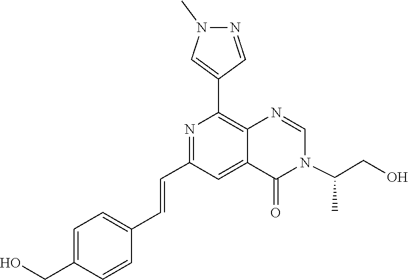 [SMILES: C[C@@H](CO)n1cnc2c(-c3cnn(C)c3)nc(/C=C/c3ccc(CO)cc3)cc2c1=O] |
| 23 | 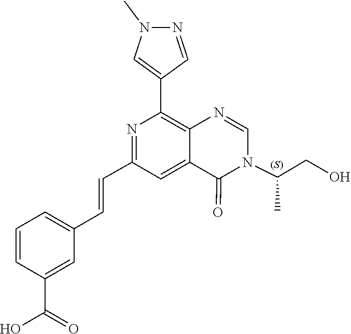 |
| 24 | 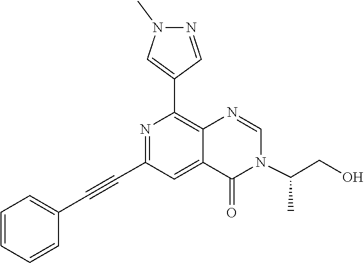 [SMILES: C[C@@H](CO)n1cnc2c(-c3cnn(C)c3)nc(C#Cc3ccccc3)cc2c1=O] |
| 25 | 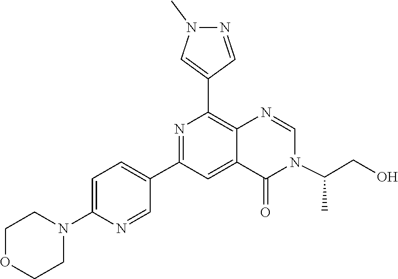 [SMILES: C[C@@H](CO)n1cnc2c(-c3cnn(C)c3)nc(-c3ccc(N4CCOCC4)nc3)cc2c1=O] |
| 26 | 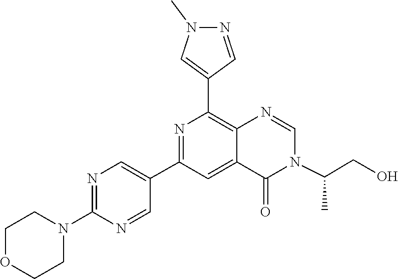 [SMILES: C[C@@H](CO)n1cnc2c(-c3cnn(C)c3)nc(-c3cnc(N4CCOCC4)nc3)cc2c1=O] |
| 27 | 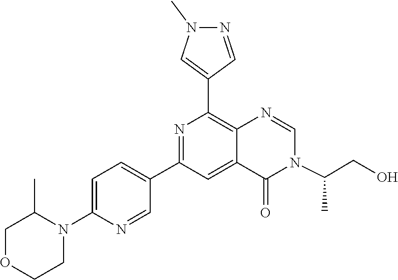 [SMILES: CC1COCCN1c1ccc(-c2cc3c(=O)n([C@@H](C)CO)cnc3c(-c3cnn(C)c3)n2)cn1] |
| 28 | 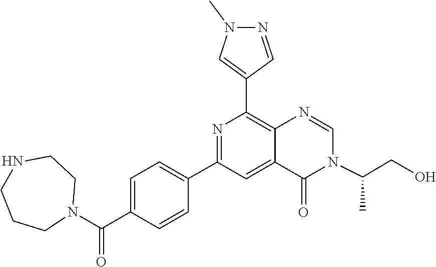 [SMILES: C[C@@H](CO)n1cnc2c(-c3cnn(C)c3)nc(-c3ccc(C(=O)N4CCCNCC4)cc3)cc2c1=O] |
| 29 | 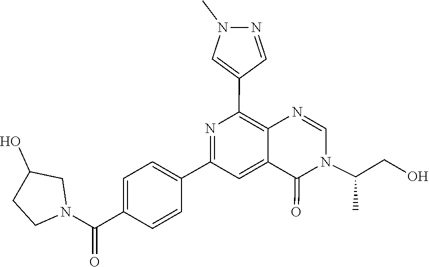 [SMILES: C[C@@H](CO)n1cnc2c(-c3cnn(C)c3)nc(-c3ccc(C(=O)N4CCC(O)C4)cc3)cc2c1=O] |
| 30 | 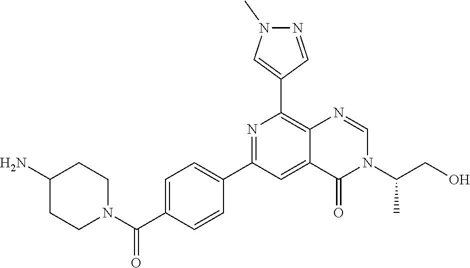 [SMILES: C[C@@H](CO)n1cnc2c(-c3cnn(C)c3)nc(-c3ccc(C(=O)N4CCC(N)CC4)cc3)cc2c1=O] |
| 31 | 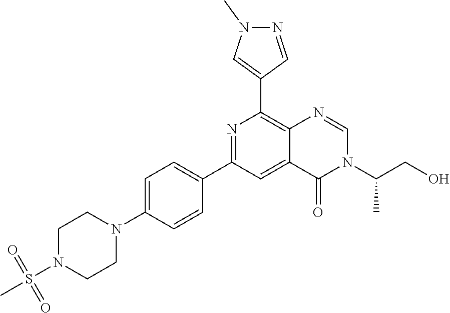 [SMILES: C[C@@H](CO)n1cnc2c(-c3cnn(C)c3)nc(-c3ccc(N4CCN(S(C)(=O)=O)CC4)cc3)cc2c1=O] |
| 32 | 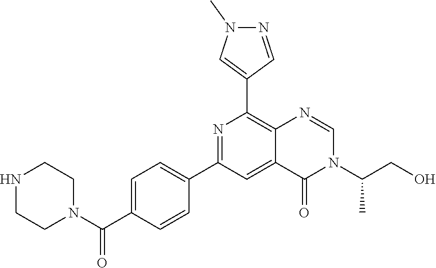 [SMILES: C[C@@H](CO)n1cnc2c(-c3cnn(C)c3)nc(-c3ccc(C(=O)N4CCNCC4)cc3)cc2c1=O] |
| 33 | 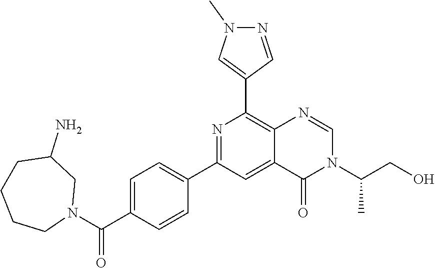 [SMILES: C[C@@H](CO)n1cnc2c(-c3cnn(C)c3)nc(-c3ccc(C(=O)N4CCCCC(N)C4)cc3)cc2c1=O] |
| 34 | 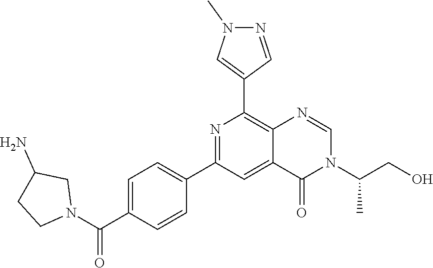 [SMILES: C[C@@H](CO)n1cnc2c(-c3cnn(C)c3)nc(-c3ccc(C(=O)N4CCC(N)C4)cc3)cc2c1=O] |
| 35 | 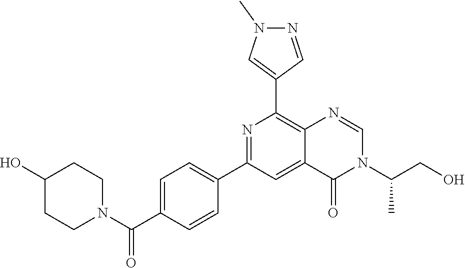 [SMILES: C[C@@H](CO)n1cnc2c(-c3cnn(C)c3)nc(-c3ccc(C(=O)N4CCC(O)CC4)cc3)cc2c1=O] |
| 36 | 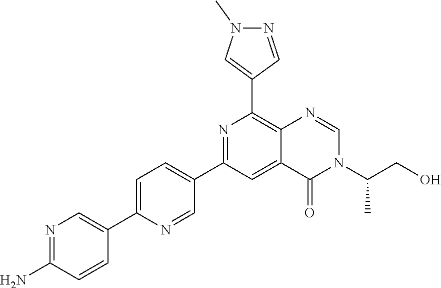 [SMILES: C[C@@H](CO)n1cnc2c(-c3cnn(C)c3)nc(-c3ccc(-c4ccc(N)nc4)nc3)cc2c1=O] |
| 37 | 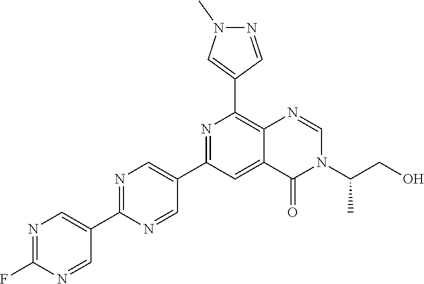 [SMILES: C[C@@H](CO)n1cnc2c(-c3cnn(C)c3)nc(-c3cnc(-c4cnc(F)nc4)nc3)cc2c1=O] |
| 38 | 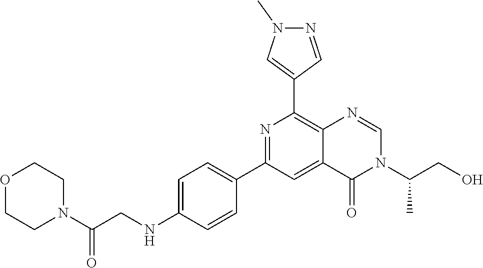 [SMILES: C[C@@H](CO)n1cnc2c(-c3cnn(C)c3)nc(-c3ccc(NCC(=O)N4CCOCC4)cc3)cc2c1=O] |
| 39 | 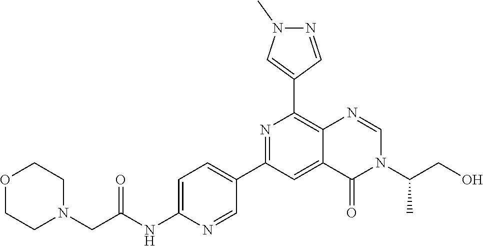 [SMILES: C[C@@H](CO)n1cnc2c(-c3cnn(C)c3)nc(-c3ccc(NC(=O)CN4CCOCC4)nc3)cc2c1=O] |
| 40 | 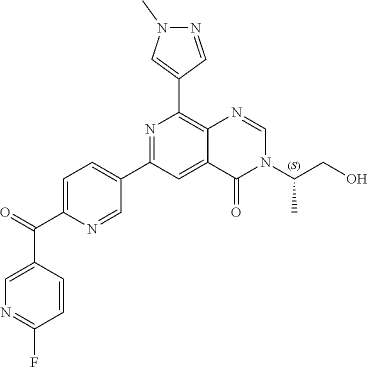 |
| 41 | 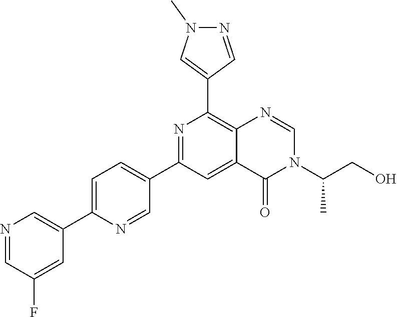 [SMILES: C[C@@H](CO)n1cnc2c(-c3cnn(C)c3)nc(-c3ccc(-c4cncc(F)c4)nc3)cc2c1=O] |
| 42 | 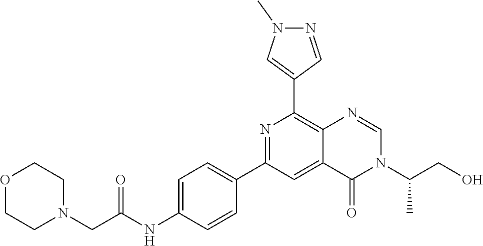 [SMILES: C[C@@H](CO)n1cnc2c(-c3cnn(C)c3)nc(-c3ccc(NC(=O)CN4CCOCC4)cc3)cc2c1=O] |
| 43 | 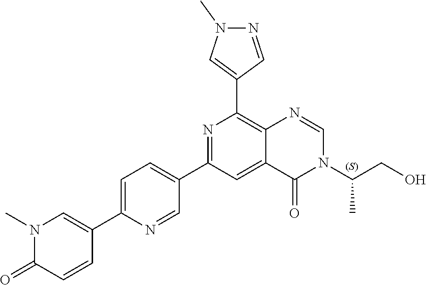 |
| 44 | 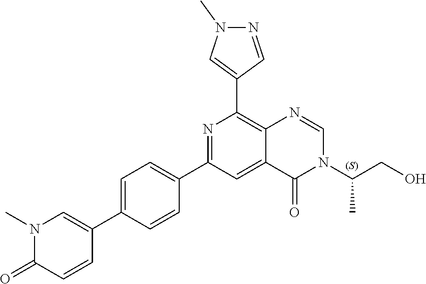 |
| 45 | 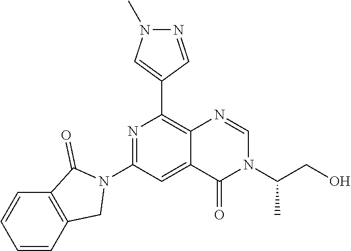 [SMILES: C[C@@H](CO)n1cnc2c(-c3cnn(C)c3)nc(N3Cc4ccccc4C3=O)cc2c1=O] |
| 46 | 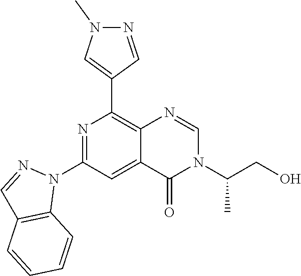 [SMILES: C[C@@H](CO)n1cnc2c(-c3cnn(C)c3)nc(-n3ncc4ccccc43)cc2c1=O] |
| 47 | 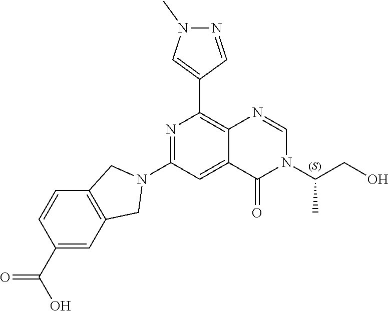 |
| 48 | 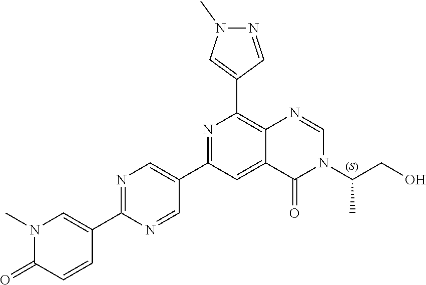 |
| 49 | 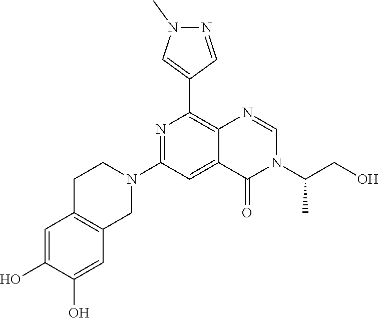 [SMILES: C[C@@H](CO)n1cnc2c(-c3cnn(C)c3)nc(N3CCc4cc(O)c(O)cc4C3)cc2c1=O] |
| 50 | 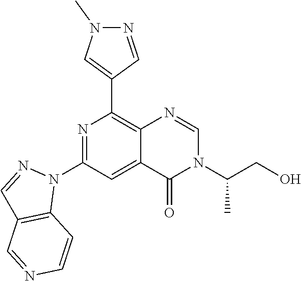 [SMILES: C[C@@H](CO)n1cnc2c(-c3cnn(C)c3)nc(-n3ncc4cnccc43)cc2c1=O] |
| 51 | 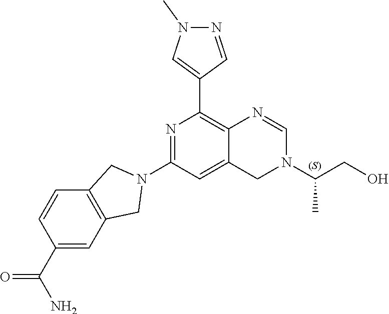 |
| 52 | 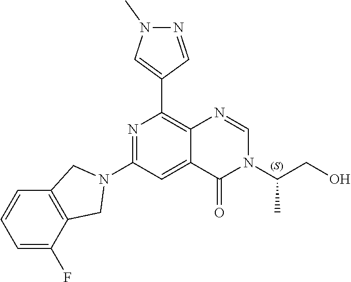 |
| 53 | 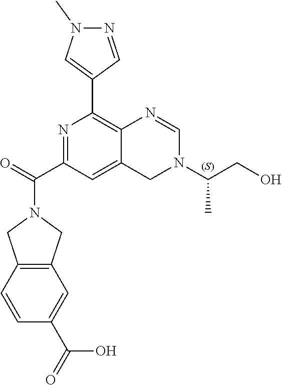 |
| 54 | 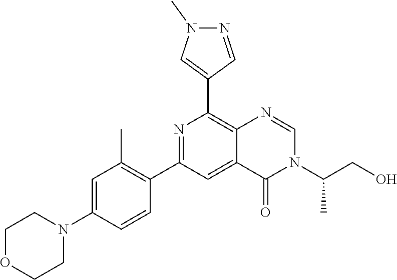 [SMILES: Cc1cc(N2CCOCC2)ccc1-c1cc2c(=O)n([C@@H](C)CO)cnc2c(-c2cnn(C)c2)n1] |
| 55 | 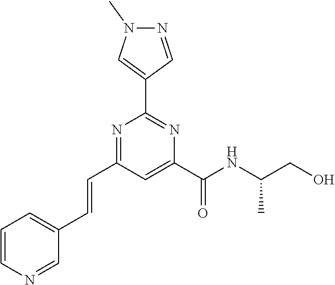 [SMILES: C[C@@H](CO)NC(=O)c1cc(/C=C/c2cccnc2)nc(-c2cnn(C)c2)n1] |
| 56 | 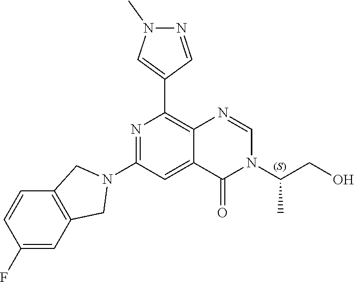 |
| 57 | 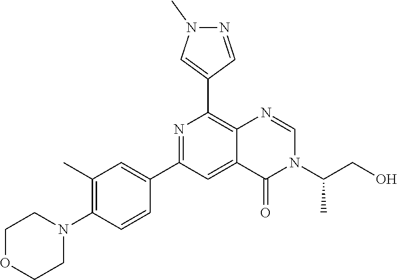 [SMILES: Cc1cc(-c2cc3c(=O)n([C@@H](C)CO)cnc3c(-c3cnn(C)c3)n2)ccc1N1CCOCC1] |
| 58 | 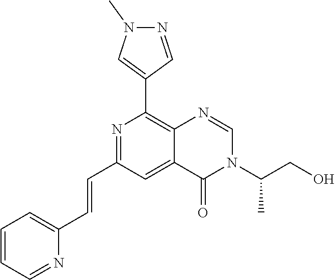 [SMILES: C[C@@H](CO)n1cnc2c(-c3cnn(C)c3)nc(/C=C/c3ccccn3)cc2c1=O] |
| 59 | 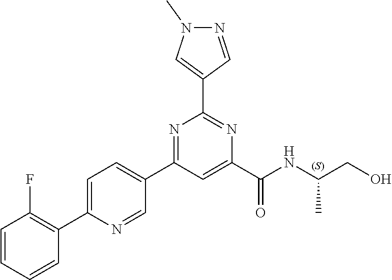 |
| 60 | 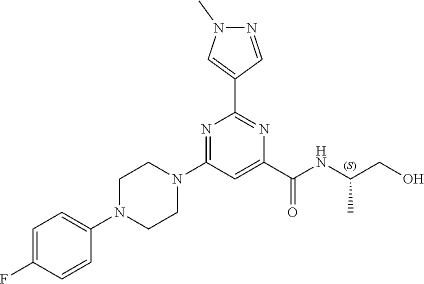 |
| 61 | 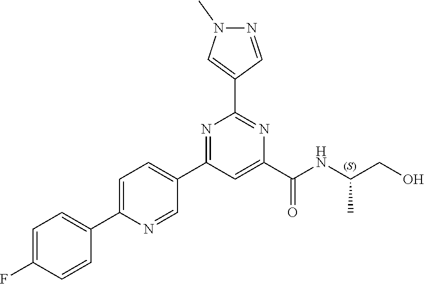 |
| 62 | 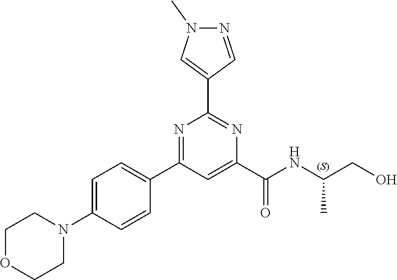 |
| 63 | 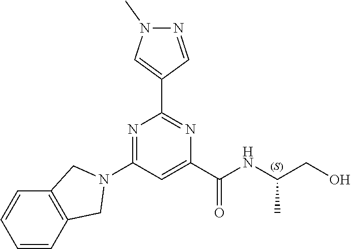 |
| 64 | 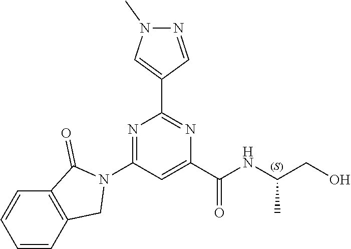 |
| 65 | 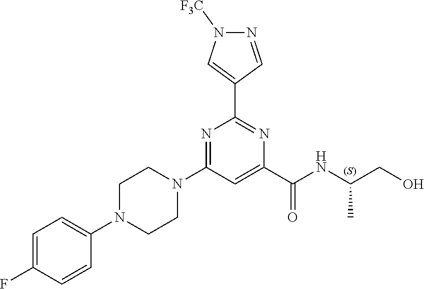 |
| 66 | 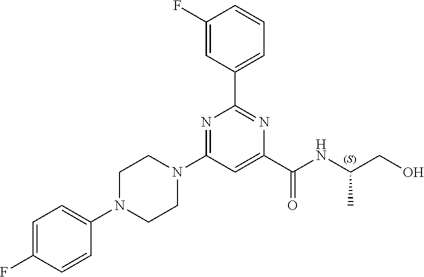 |
| 67 | 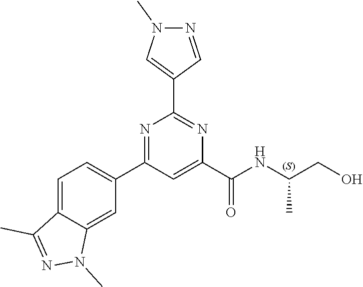 |
| 68 | 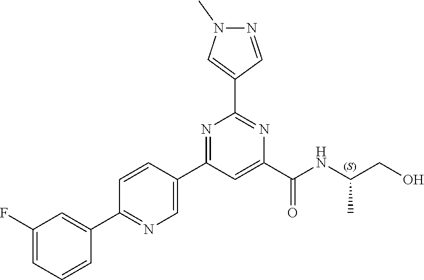 |
| 69 | 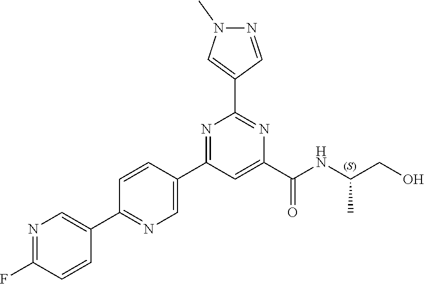 |
| 70 | 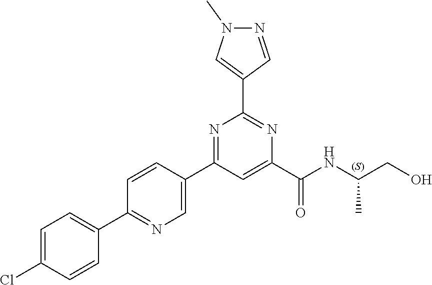 |
| 71 | 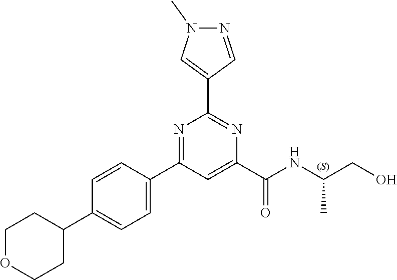 |
| 72 | 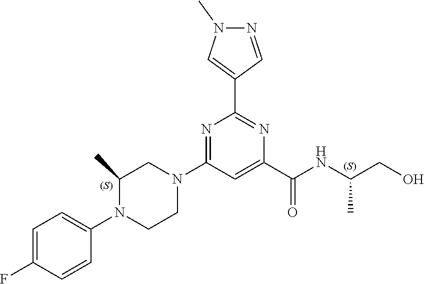 |
| 73 | 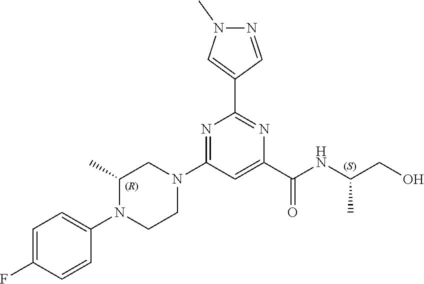 |
| 74 | 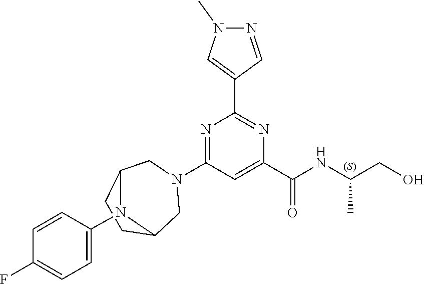 |
| 75 | 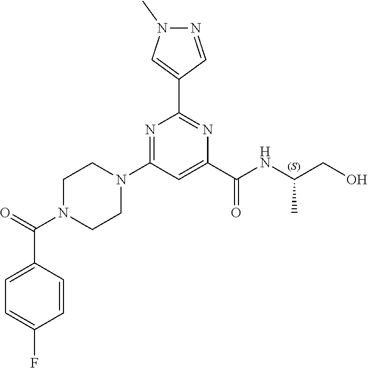 |
| 76 | 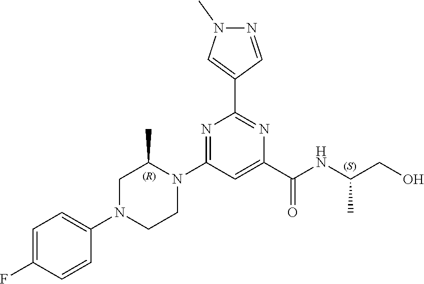 |
| 77 | 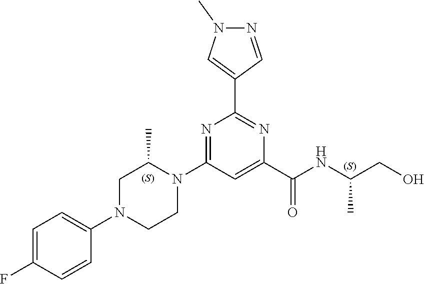 |
| 78 | 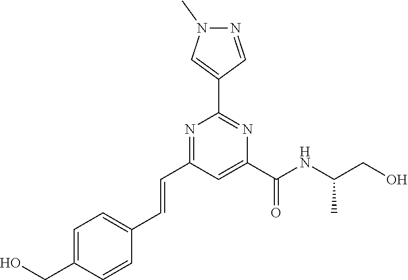 [SMILES: C[C@@H](CO)NC(=O)c1cc(/C=C/c2ccc(CO)cc2)nc(-c2cnn(C)c2)n1] |
| 79 | 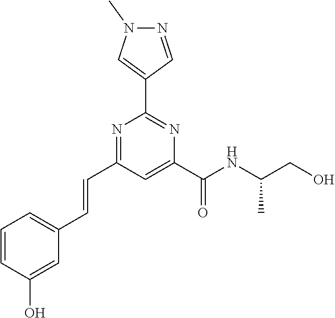 [SMILES: C[C@@H](CO)NC(=O)c1cc(/C=C/c2cccc(O)c2)nc(-c2cnn(C)c2)n1] |
| 80 | 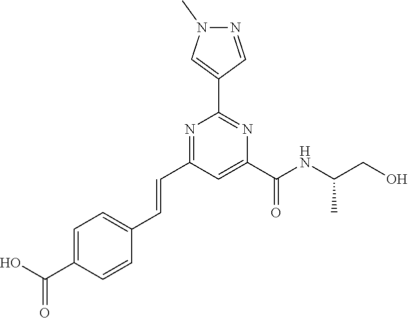 [SMILES: C[C@@H](CO)NC(=O)c1cc(/C=C/c2ccc(C(=O)O)cc2)nc(-c2cnn(C)c2)n1] |
| 81 | 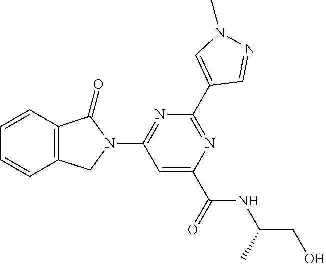 [SMILES: C[C@@H](CO)NC(=O)c1cc(N2Cc3ccccc3C2=O)nc(-c2cnn(C)c2)n1] |
| 82 | 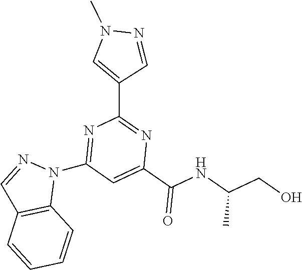 [SMILES: C[C@@H](CO)NC(=O)c1cc(-n2ncc3ccccc32)nc(-c2cnn(C)c2)n1] |
| 83 | 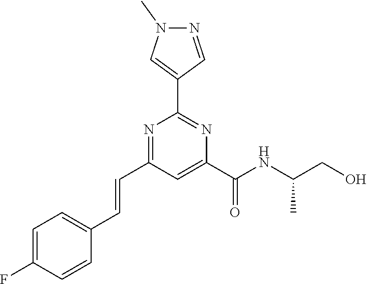 [SMILES: C[C@@H](CO)NC(=O)c1cc(/C=C/c2ccc(F)cc2)nc(-c2cnn(C)c2)n1] |
| 84 | 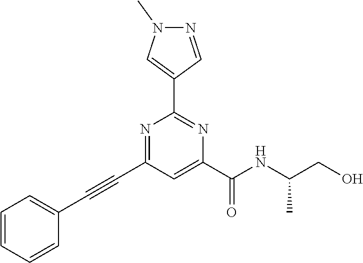 [SMILES: C[C@@H](CO)NC(=O)c1cc(C#Cc2ccccc2)nc(-c2cnn(C)c2)n1] |
| 85 | 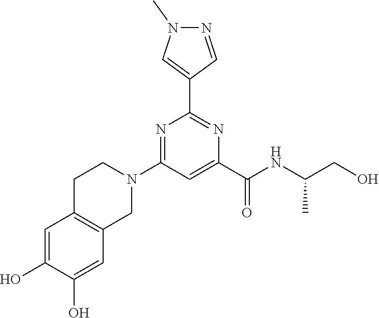 [SMILES: C[C@@H](CO)NC(=O)c1cc(N2CCc3cc(O)c(O)cc3C2)nc(-c2cnn(C)c2)n1] |
| 86 | 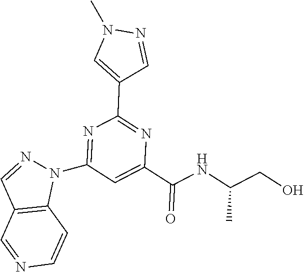 [SMILES: C[C@@H](CO)NC(=O)c1cc(-n2ncc3cnccc32)nc(-c2cnn(C)c2)n1] |
| 87 | 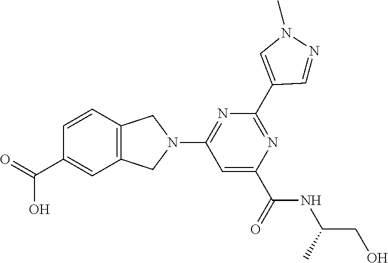 [SMILES: C[C@@H](CO)NC(=O)c1cc(N2Cc3ccc(C(=O)O)cc3C2)nc(-c2cnn(C)c2)n1] |
| 88 | 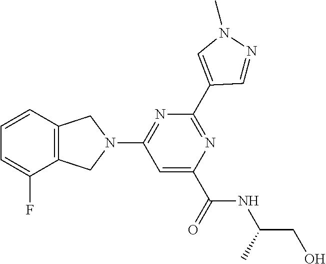 [SMILES: C[C@@H](CO)NC(=O)c1cc(N2Cc3cccc(F)c3C2)nc(-c2cnn(C)c2)n1] |
| 89 | 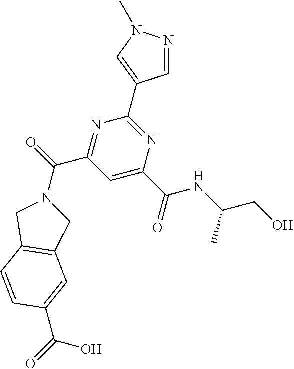 [SMILES: C[C@@H](CO)NC(=O)c1cc(C(=O)N2Cc3ccc(C(=O)O)cc3C2)nc(-c2cnn(C)c2)n1] |
| 90 | 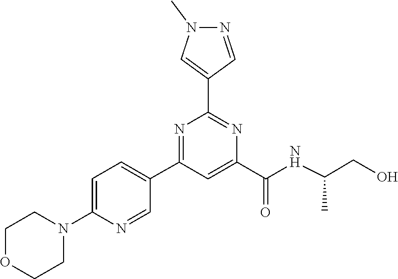 [SMILES: C[C@@H](CO)NC(=O)c1cc(-c2ccc(N3CCOCC3)nc2)nc(-c2cnn(C)c2)n1] |
| 91 | 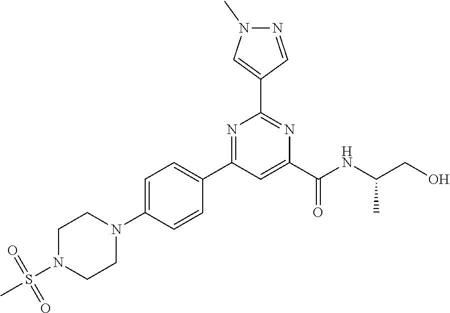 [SMILES: C[C@@H](CO)NC(=O)c1cc(-c2ccc(N3CCN(S(C)(=O)=O)CC3)cc2)nc(-c2cnn(C)c2)n1] |
| 92 | 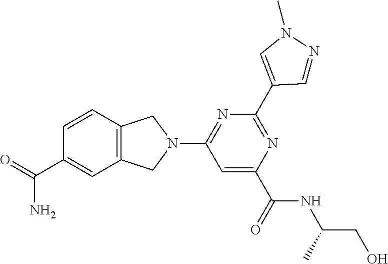 [SMILES: C[C@@H](CO)NC(=O)c1cc(N2Cc3ccc(C(N)=O)cc3C2)nc(-c2cnn(C)c2)n1] |
| 93 | 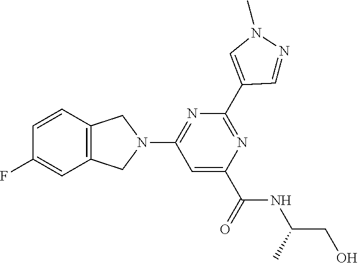 [SMILES: C[C@@H](CO)NC(=O)c1cc(N2Cc3ccc(F)cc3C2)nc(-c2cnn(C)c2)n1] |
| 94 | 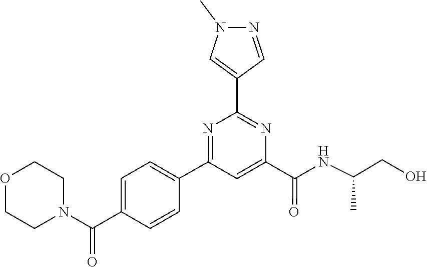 [SMILES: C[C@@H](CO)NC(=O)c1cc(-c2ccc(C(=O)N3CCOCC3)cc2)nc(-c2cnn(C)c2)n1] |
| 95 | 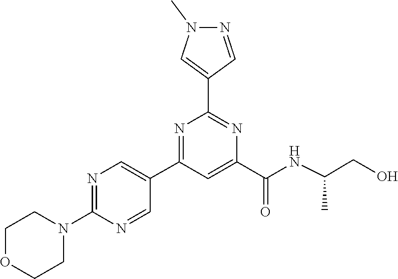 [SMILES: C[C@@H](CO)NC(=O)c1cc(-c2cnc(N3CCOCC3)nc2)nc(-c2cnn(C)c2)n1] |
| 96 | 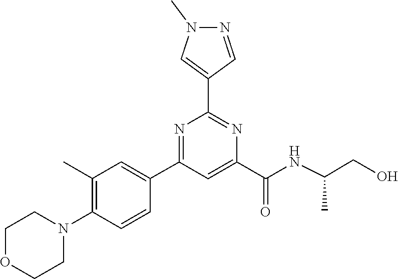 [SMILES: Cc1cc(-c2cc(C(=O)N[C@@H](C)CO)nc(-c3cnn(C)c3)n2)ccc1N1CCOCC1] |
| 97 | 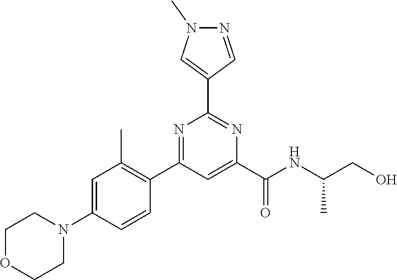 [SMILES: Cc1cc(N2CCOCC2)ccc1-c1cc(C(=O)N[C@@H](C)CO)nc(-c2cnn(C)c2)n1] |
| 98 | 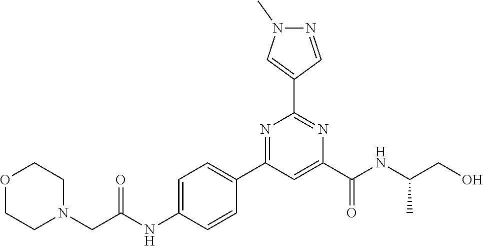 [SMILES: C[C@@H](CO)NC(=O)c1cc(-c2ccc(NC(=O)CN3CCOCC3)cc2)nc(-c2cnn(C)c2)n1] |
| 99 | 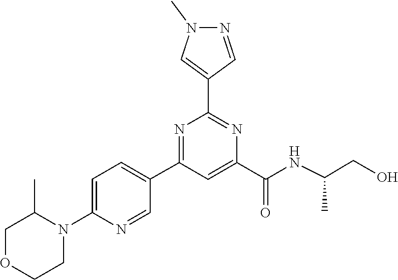 [SMILES: CC1COCCN1c1ccc(-c2cc(C(=O)N[C@@H](C)CO)nc(-c3cnn(C)c3)n2)cn1] |
| 100 | 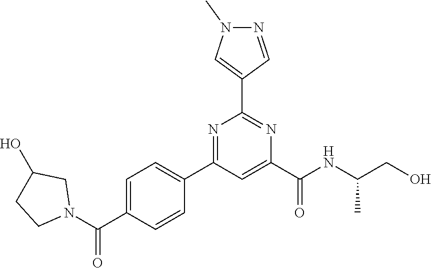 [SMILES: C[C@@H](CO)NC(=O)c1cc(-c2ccc(C(=O)N3CCC(O)C3)cc2)nc(-c2cnn(C)c2)n1] |
| 101 | 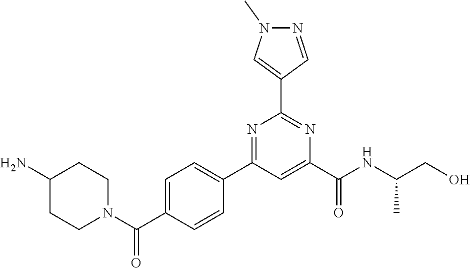 [SMILES: C[C@@H](CO)NC(=O)c1cc(-c2ccc(C(=O)N3CCC(N)CC3)cc2)nc(-c2cnn(C)c2)n1] |
| 102 | 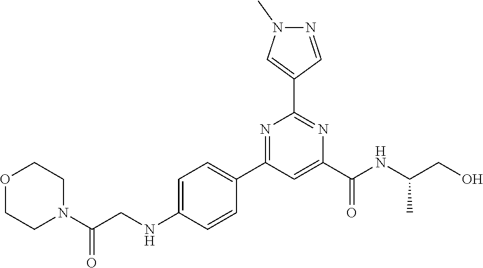 [SMILES: C[C@@H](CO)NC(=O)c1cc(-c2ccc(NCC(=O)N3CCOCC3)cc2)nc(-c2cnn(C)c2)n1] |
| 103 | 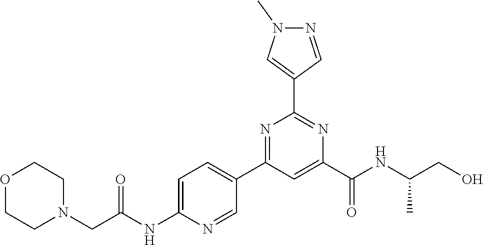 [SMILES: C[C@@H](CO)NC(=O)c1cc(-c2ccc(NC(=O)CN3CCOCC3)nc2)nc(-c2cnn(C)c2)n1] |
| 104 | 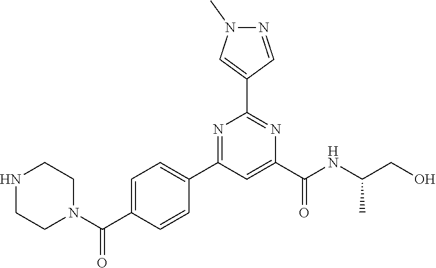 [SMILES: C[C@@H](CO)NC(=O)c1cc(-c2ccc(C(=O)N3CCNCC3)cc2)nc(-c2cnn(C)c2)n1] |
| 105 | 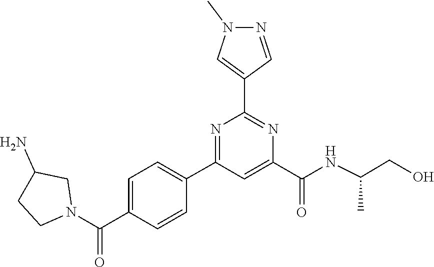 [SMILES: C[C@@H](CO)NC(=O)c1cc(-c2ccc(C(=O)N3CCC(N)C3)cc2)nc(-c2cnn(C)c2)n1] |
| 106 | 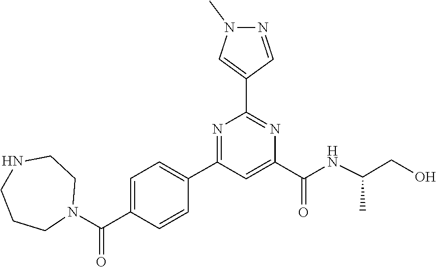 [SMILES: C[C@@H](CO)NC(=O)c1cc(-c2ccc(C(=O)N3CCCNCC3)cc2)nc(-c2cnn(C)c2)n1] |
| 107 | 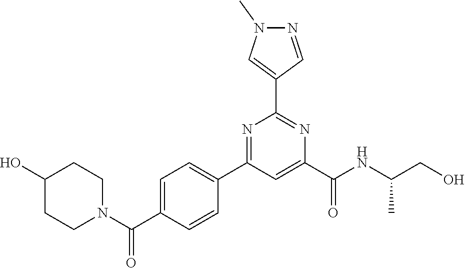 [SMILES: C[C@@H](CO)NC(=O)c1cc(-c2ccc(C(=O)N3CCC(O)CC3)cc2)nc(-c2cnn(C)c2)n1] |
| 108 | 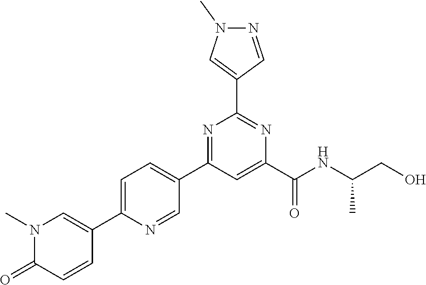 [SMILES: C[C@@H](CO)NC(=O)c1cc(-c2ccc(-c3ccc(=O)n(C)c3)nc2)nc(-c2cnn(C)c2)n1] |
| 109 | 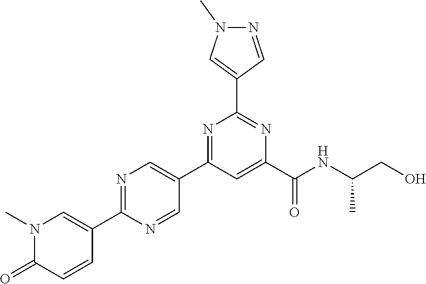 [SMILES: C[C@@H](CO)NC(=O)c1cc(-c2cnc(-c3ccc(=O)n(C)c3)nc2)nc(-c2cnn(C)c2)n1] |
| 110 | 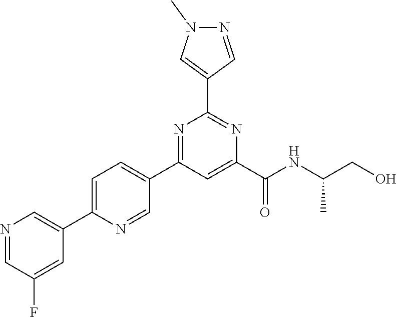 [SMILES: C[C@@H](CO)NC(=O)c1cc(-c2ccc(-c3cncc(F)c3)nc2)nc(-c2cnn(C)c2)n1] |
| 111 | 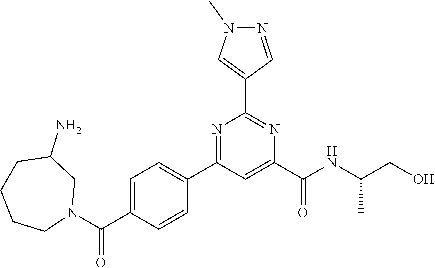 [SMILES: C[C@@H](CO)NC(=O)c1cc(-c2ccc(C(=O)N3CCCCC(N)C3)cc2)nc(-c2cnn(C)c2)n1] |
| 112 | 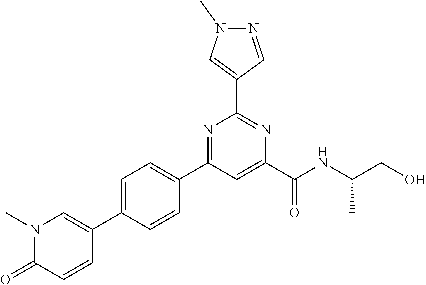 [SMILES: C[C@@H](CO)NC(=O)c1cc(-c2ccc(-c3ccc(=O)n(C)c3)cc2)nc(-c2cnn(C)c2)n1] |
| 113 | 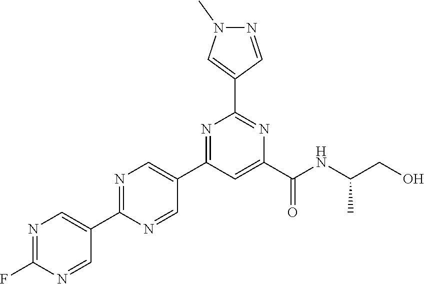 [SMILES: C[C@@H](CO)NC(=O)c1cc(-c2cnc(-c3cnc(F)nc3)nc2)nc(-c2cnn(C)c2)n1] |
| 114 | 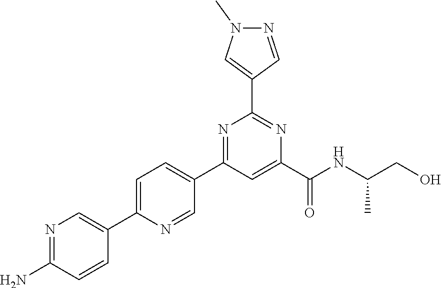 [SMILES: C[C@@H](CO)NC(=O)c1cc(-c2ccc(-c3ccc(N)nc3)nc2)nc(-c2cnn(C)c2)n1] |
| 115 | 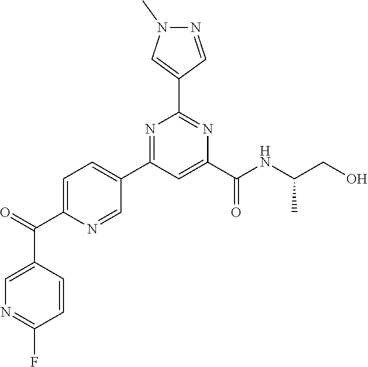 [SMILES: C[C@@H](CO)NC(=O)c1cc(-c2ccc(C(=O)c3ccc(F)nc3)nc2)nc(-c2cnn(C)c2)n1] |
| 116 | 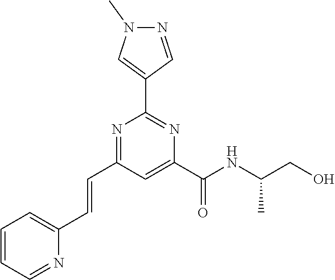 [SMILES: C[C@@H](CO)NC(=O)c1cc(/C=C/c2ccccn2)nc(-c2cnn(C)c2)n1] |
| 117 | 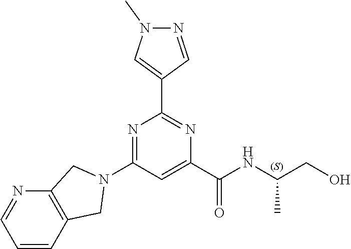 |
| 118 | 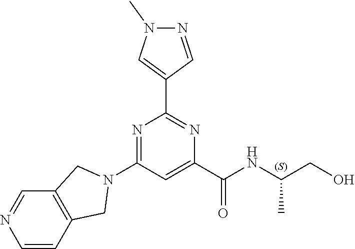 |
| 119 | 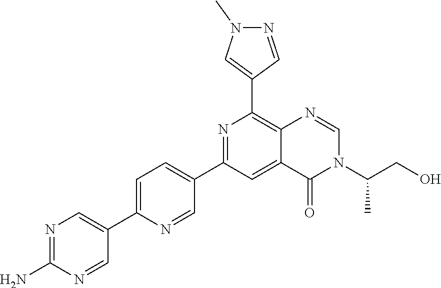 [SMILES: C[C@@H](CO)n1cnc2c(-c3cnn(C)c3)nc(-c3ccc(-c4cnc(N)nc4)nc3)cc2c1=O] |
| 120 | 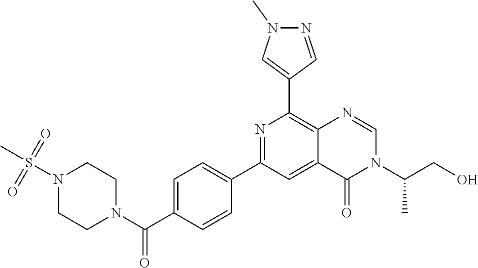 [SMILES: C[C@@H](CO)n1cnc2c(-c3cnn(C)c3)nc(-c3ccc(C(=O)N4CCN(S(C)(=O)=O)CC4)cc3)cc2c1=O] |
| 121 | 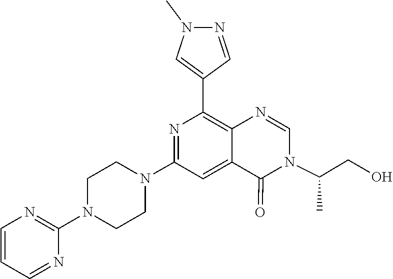 [SMILES: C[C@@H](CO)n1cnc2c(-c3cnn(C)c3)nc(N3CCN(c4ncccn4)CC3)cc2c1=O] |
| 122 | 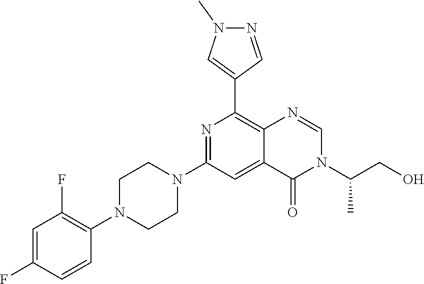 [SMILES: C[C@@H](CO)n1cnc2c(-c3cnn(C)c3)nc(N3CCN(c4ccc(F)cc4F)CC3)cc2c1=O] |
| 123 | 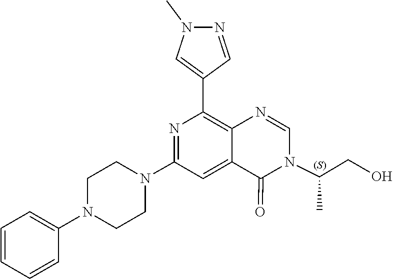 |
| 124 | 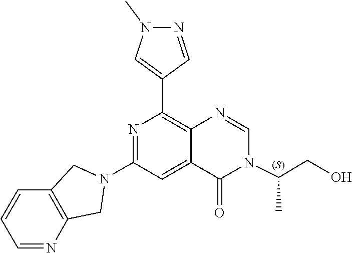 |
| 125 | 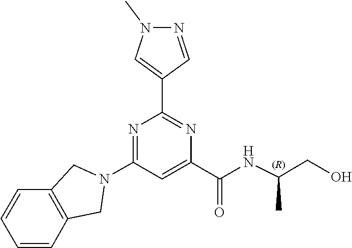 |
| 126 | 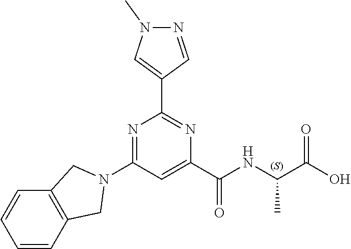 |
| 127 | 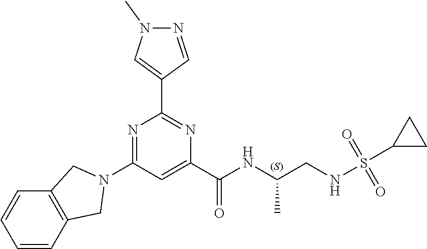 |
| 128 | 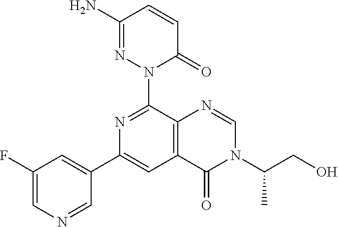 [SMILES: C[C@@H](CO)n1cnc2c(-n3nc(N)ccc3=O)nc(-c3cncc(F)c3)cc2c1=O] |
| 129 | 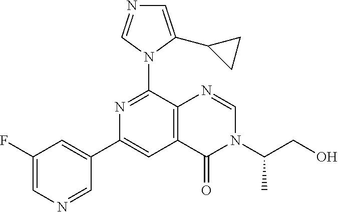 [SMILES: C[C@@H](CO)n1cnc2c(-n3cncc3C3CC3)nc(-c3cncc(F)c3)cc2c1=O] |

Unless otherwise indicated, structures depicted herein are also meant to include all isomeric (e.g., enantiomeric, diastereomeric, cis-trans, conformational, and rotational) forms of the structure. For example, the R and S configurations for each asymmetric center, (Z) and (E) double bond isomers, and (Z) and (E) conformational isomers are included in this disclosure, unless only one of the isomers is specifically indicated. Therefore, single stereochemical isomers as well as enantiomeric, diastereomeric, cis/trans, conformational, and rotational mixtures of the present compounds are within the scope of the disclosure. In some cases, the compounds disclosed herein are stereoisomers. “Stereoisomers” refer to compounds that differ in the chirality of one or more stereocenters. Stereoisomers include enantiomers and diastereomers. The compounds disclosed herein can exist as a single stereoisomer, or as a mixture of stereoisomers. Stereochemistry of the compounds shown herein indicate a relative stereochemistry, not absolute, unless discussed otherwise. As indicated herein, a single stereoisomer, diastereomer, or enantiomer refers to a compound that is at least more than 50% of the indicated stereoisomer, diastereomer, or enantiomer, and in some cases, at least 90% or 95% of the indicated stereoisomer, diastereomer, or enantiomer.

The compounds disclosed herein can have any stereochemical configuration at the sp³ carbon atoms. In some cases, the compounds of the disclosure are optically pure. As used herein, “optically pure” refers to the presence of only one enantiomer (to limits of detection) of a compound if multiple stereochemical configurations can exist. In various cases, the chiral moieties present in the compounds of the disclosure are derived from either natural or unnatural amino acids or saccharides.

Unless otherwise indicated, all tautomeric forms of the compounds of the disclosure are within the scope of the disclosure.

Additionally, unless otherwise indicated, structures depicted herein are also meant to include compounds that differ only in the presence of one or more isotopically enriched atoms. For example, compounds having the present structures except for the replacement of hydrogen by deuterium or tritium, or the replacement of a carbon by a ¹³C- or ¹⁴C-enriched carbon are within the scope of this disclosure. Such compounds are useful, for example, as analytical tools or probes in biological assays. Such compounds, especially deuterium analogs, can also be therapeutically useful. Thus, further disclosed herein are deuterated compounds or salts of Formula (I), in which one or more isotopes of hydrogen have been replaced with deuterium.

The compounds of the disclosure are defined herein by their chemical structures and/or chemical names. Where a compound is referred to by both a chemical structure and a chemical name, and the chemical structure and chemical name conflict, the chemical structure is determinative of the compound's identity.

As used herein, “alkyl” refers to straight chained and branched saturated hydrocarbon groups containing one to thirty carbon atoms, for example, one to six carbon atoms (e.g., 1, 2, 3, 4, 5, or 6). The term Cₙ means the alkyl group has “n” carbon atoms. For example, C₃ alkyl refers to an alkyl group that has 3 carbon atoms. C₁₋₆alkyl refers to an alkyl group having a number of carbon atoms encompassing the entire range (i.e., 1 to 6 carbon atoms), as well as all subgroups (e.g., 1-2, 1-3, 1-4, 1-5, 1-6, 2-3, 2-4, 2-5, 2-6, 3-4, 3-5, 3-6, 4-5, 4-6, 5-6, 1, 2, 3, 4, 5, and 6 carbon atoms). Nonlimiting examples of alkyl groups include, methyl, ethyl, n-propyl, isopropyl, n-butyl, sec-butyl(2-methylpropyl), and t-butyl (1,1-dimethylethyl). Unless otherwise indicated, an alkyl group can be an unsubstituted alkyl group or a substituted alkyl group.

As used herein, “alkylene” refers to a bivalent saturated aliphatic radical. The term Cₙ means the alkylene group has “n” carbon atoms. For example, C₁₋₆alkylene refers to an alkylene group having a number of carbon atoms encompassing the entire range, as well as all subgroups, as previously described for “alkyl” groups.

As used herein, the term “haloalkyl” refers to an alkyl group in which one or more of the hydrogen atoms are replaced by halogen. Such groups include but are not limited to, chloromethyl, fluoromethyl, difluoromethyl, trifluoromethyl, 1,1-difluoroethyl, 2-fluoroethyl, 1-chloro-2-fluoromethyl and 2-fluoroisobutyl. A haloalkyl may be further substituted or unsubstituted, and some cases relate to a haloalkyl having e.g., 1 to 6 carbon atoms, such as C₁-6 haloalkyl.

As used herein, the term “hydroxyalkyl” refers to an alkyl group in which one or more of the hydrogen atoms are replaced by a hydroxyl group (OH). Such groups include but are not limited to, hydroxymethyl, hydroxyethyl, and the like. A hydroxyalkyl may be further substituted or unsubstituted, and some cases relate to a hydroxyalkyl having e.g., 1 to 6 carbon atoms, such as C₁₋₆ hydroxyalkyl.

As used herein, the term “cycloalkyl” refers to an aliphatic cyclic hydrocarbon group containing three to eight carbon atoms (e.g., 3, 4, 5, 6, 7, or 8 carbon atoms). The term Cₙ means the cycloalkyl group has “n” carbon atoms. For example, C₅ cycloalkyl refers to a cycloalkyl group that has 5 carbon atoms in the ring. C₃-C₈ cycloalkyl refers to cycloalkyl groups having a number of carbon atoms encompassing the entire range (e.g., 3 to 8 carbon atoms), as well as all subgroups (e.g., 3-4, 3-5, 3-6, 3-7, 3-8, 4-5, 4-6, 4-7, 4-8, 5-6, 5-7, 5-8, 6-7, 6-8, 7-8, 3, 4, 5, 6, 7, and 8 carbon atoms). Nonlimiting examples of cycloalkyl groups include cyclopropyl, cyclobutyl, cyclopentyl, cyclohexyl, cycloheptyl, and cyclooctyl. The cycloalkyl groups described herein can be isolated or fused to another cycloalkyl group, a heterocycloalkyl group, an aryl group and/or a heteroaryl group. When a cycloalkyl group is fused to another cycloalkyl group, then each of the cycloalkyl groups can contain three to eight carbon atoms unless specified otherwise. Unless otherwise indicated, a cycloalkyl group can be unsubstituted or substituted.

The term “heterocyclyl” as used herein refers to a monocyclic, fused, spiro or bridged ring system which can be saturated or contain one or more units of unsaturation or be aromatic (i.e., heteroaryl), having 4 to 12 (or 4 to 10 or 4 to 8, e.g., as specified in the disclosure) total ring atoms in which 1-3 (e.g., one to three, or one, two, or three) ring atoms is a heteroatom selected from, N, S, and O. In cases where the heterocyclyl is spiro, the group can comprise, e.g., a 4-membered ring and spiro 5-membered ring; a 4-membered ring and spiro 6-membered ring; a 4-membered ring and spiro 7-membered ring; a 5-membered ring and spiro 6-membered ring; a 5-membered ring and spiro 7-membered ring; a 6-membered ring and spiro 6-membered ring; a 6-membered ring and spiro 7-membered ring; or a 7-membered ring and spiro 7-membered ring; and each ring of the spiro system may contain at least one ring heteroatom, or only one ring may contain at least one ring heteroatom and the other ring can be carbocyclic. In some cases, the heterocyclyl comprises 5-6 ring members. In some cases, the heterocyclyl comprises 5 ring members. In some cases, the heterocyclyl comprises 6 ring members. Examples of heterocyclyl groups include, but are not limited to, oxetanyl, azetidinyl, piperidinyl, piperazinyl, pyrrolidinyl, pyrazolidinyl, imidazolidinyl, azepanyl, diazepanyl, triazepanyl, oxazolidinyl, isoxazolidinyl, thiazolidinyl, isothiazolidinyl, oxazepanyl, thiazepanyl, tetrahydrofuranyl, tetrahydrothiophenyl, morpholino (including, for example, 3-morpholino, 4-morpholino), 2-thiomorpholino, 3-thiomorpholino, 4-thiomorpholino, 1-pyrrolidinyl, 2-pyrrolidinyl, 3-pyrrolidinyl, pyrrolidin-2-one, 1-tetrahydropiperazinyl, 2-tetrahydropiperazinyl, 3-tetrahydropiperazinyl, 1-piperidinyl, 2-piperidinyl, 3-piperidinyl, 1-pyrazolinyl, 3-pyrazolinyl, 4-pyrazolinyl, 5-pyrazolinyl, 1-piperidinyl, 2-piperidinyl, 3-piperidinyl, 4-piperidinyl, 2-thiazolidinyl, 3-thiazolidinyl, 4-thiazolidinyl, 1-imidazolidinyl, 2-imidazolidinyl, 4-imidazolidinyl, 5-imidazolidinyl, and 1,3-dihydro-imidazol-2-onyl. In some cases, heterocyclyl is oxetanyl, azetidinyl, diazepanyl (such as 1,4-diazepanyl), azepanyl, piperidinyl (such as 1-piperidinyl, 2-piperidinyl, 3-piperidinyl, or 4-piperidinyl), or pyrrolidinyl (such as 1-pyrrolidinyl, 2-pyrrolidinyl, or 3-pyrrolidinyl). In some cases, heterocyclyl is oxetanyl, azetidinyl, diazepanyl or azepanyl. In some cases, heterocyclyl is diazepanyl or azepanyl. In some cases, heterocyclyl is 1,4-diazepanyl or azepanyl. In some cases, heterocyclyl is 1,4-diazepanyl. In some cases, heterocyclyl is azepanyl. A heterocyclyl ring is unsubstituted or substituted as described herein.

As used herein, the term “aromatic ring” refers to a monocyclic or bicyclic aromatic group, having 5 to 10 ring atoms, and be all carbon ring atoms (i.e., aryl) or can include 1-3 ring heteroatoms (i.e., heteroaryl). Unless otherwise indicated, an aromatic ring can be unsubstituted or substituted. Aromatic rings can be unsubstituted or substituted as described elsewhere herein.

As used herein, the term “heteroaryl” refers to a heterocycyl group that is aromatic, having five to twelve total ring atoms (e.g., a monocyclic aromatic ring with 5-6 total ring atoms), and containing 1-3 heteroatoms selected from nitrogen, oxygen, and sulfur atom in the aromatic ring. Unless otherwise indicated, a heteroaryl group can be unsubstituted or substituted. Heteroaryl groups can be isolated (e.g., pyridyl) or fused to another heteroaryl group (e.g., purinyl), a cycloalkyl group (e.g., tetrahydroquinolinyl), a non-aromatic heterocyclyl group (e.g., dihydronaphthyridinyl), and/or an aryl group (e.g., benzothiazolyl and quinolyl). Examples of heteroaryl groups include, but are not limited to, pyrazolyl, thienyl, furyl, pyridyl, pyrrolyl, oxazolyl, quinolyl, thiophenyl, isoquinolyl, indolyl, triazinyl, triazolyl, isothiazolyl, isoxazolyl, imidazolyl, benzothiazolyl, pyrazinyl, pyrimidinyl, thiazolyl, and thiadiazolyl. When a heteroaryl group is fused to another heteroaryl group, then each ring can contain five or six total ring atoms and one to three heteroatoms in its aromatic ring.

As used herein, the term “alkoxy” as used herein refers to a “—O-alkyl” group.

As used herein, the term “haloalkoxy” as used herein refers to a “—O-haloalkyl” group.

As used herein, the term “halo” refers to a fluoro (F), chloro (Cl), bromo (Br), or iodo (I) group.

A “substituted” functional group (e.g., a substituted heterocyclyl, aromatic ring, or heteroaryl) is a functional, group having at least one hydrogen radical that is substituted with a non-hydrogen radical (i.e., a substituent). Examples of non-hydrogen radicals (or substituents) include, but are not limited to, alkyl, cycloalkyl, alkenyl, cycloalkenyl, alkynyl, ether, aryl, heteroaryl, heterocycloalkyl, hydroxyl, oxy (or oxo), alkoxyl, ester, thioester, acyl, carboxyl, cyano, nitro, amino, sulfhydryl, and halo. Substitutions are discussed in relation to specific functional groups or moieties herein.

### Pharmaceutically Acceptable Salts

The compounds described herein can exist in free form, or where appropriate, as salts. Those salts that are pharmaceutically acceptable are of particular interest since they are useful in administering the compounds described herein for medical purposes. Salts that are not pharmaceutically acceptable are useful in manufacturing processes, for isolation and purification purposes, and in some cases, for use in separating stereoisomeric forms of the compounds of the disclosure or intermediates thereof.

As used herein, the term “pharmaceutically acceptable salt” refers to salts of a compound which are, within the scope of sound medical judgment, suitable for use in contact with the tissues of humans and lower animals without undue side effects, such as, toxicity, irritation, allergic response and the like, and are commensurate with a reasonable benefit/risk ratio.

Pharmaceutically acceptable salts are well known in the art. For example, S. M. Berge et al., describe pharmaceutically acceptable salts in detail in J. Pharmaceutical Sciences, 1977, 66, 1-19, which is incorporated herein by reference. Pharmaceutically acceptable salts of the compounds described herein include those derived from suitable inorganic and organic acids and bases. These salts can be prepared in situ during the final isolation and purification of the compounds.

Where the compound described herein contains a basic group, or a sufficiently basic bioisostere, acid addition salts can be prepared by 1) reacting the purified compound in its free-base form with a suitable organic or inorganic acid and 2) isolating the salt thus formed. In practice, acid addition salts might be a more convenient form for use and use of the salt amounts to use of the free basic form.

Examples of pharmaceutically acceptable, non-toxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, glycolate, gluconate, glycolate, hemisulfate, heptanoate, hexanoate, hydrochloride, hydrobromide, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, palmoate, pectinate, persulfate, 3-phenylpropionate, phosphate, picrate, pivalate, propionate, salicylate, stearate, succinate, sulfate, tartrate, thiocyanate, p-toluenesulfonate, undecanoate, valerate salts, and the like.

Where the compound described herein contains a carboxyl group or a sufficiently acidic bioisostere, base addition salts can be prepared by 1) reacting the purified compound in its acid form with a suitable organic or inorganic base and 2) isolating the salt thus formed. In practice, use of the base addition salt might be more convenient and use of the salt form inherently amounts to use of the free acid form. Salts derived from appropriate bases include alkali metal (e.g., sodium, lithium, and potassium), alkaline earth metal (e.g., magnesium and calcium), ammonium and N⁺(C₁₋₄alkyl)₄ salts. This disclosure also envisions the quaternization of any basic nitrogen-containing groups of the compounds disclosed herein. Water or oil-soluble or dispersible products may be obtained by such quaternization.

Basic addition salts include pharmaceutically acceptable metal and amine salts. Suitable metal salts include the sodium, potassium, calcium, barium, zinc, magnesium, and aluminum. The sodium and potassium salts are usually preferred. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Suitable inorganic base addition salts are prepared from metal bases which include sodium hydride, sodium hydroxide, potassium hydroxide, calcium hydroxide, aluminum hydroxide, lithium hydroxide, magnesium hydroxide, zinc hydroxide and the like. Suitable amine base addition salts are prepared from amines which are frequently used in medicinal chemistry because of their low toxicity and acceptability for medical use. Ammonia, ethylenediamine, N-methyl-glucamine, lysine, arginine, ornithine, choline, N,N′-dibenzylethylenediamine, chloroprocaine, dietanolamine, procaine, N-benzylphenethylamine, diethylamine, tris(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, dicyclohexylamine and the like.

Other acids and bases, although not in themselves pharmaceutically acceptable, may be employed in the preparation of salts useful as intermediates in obtaining the compounds described herein and their pharmaceutically acceptable acid or base addition salts.

It should be understood that a compound disclosed herein can be present as a mixture/combination of different pharmaceutically acceptable salts. Also contemplated are mixtures/combinations of compounds in free form and pharmaceutically acceptable salts.

### Pharmaceutical Formulations

Also provided herein are pharmaceutical formulations that include an effective amount of compounds of the disclosure and one or more pharmaceutically acceptable excipients. As used herein, the term “formulation” is used interchangeable with “composition.”

An “effective amount” includes a “therapeutically effective amount” and a “prophylactically effective amount.” The term “therapeutically effective amount” refers to an amount effective in treating and/or ameliorating a disease or condition in a subject. The term “prophylactically effective amount” refers to an amount effective in preventing and/or substantially lessening the chances of a disease or condition in a subject. As used herein, the terms “patient” and “subject” may be used interchangeably and mean animals, such as dogs, cats, cows, horses, and sheep (i.e., non-human animals) and humans. Particular patients or subjects are mammals (e.g., humans). The terms “patient” and “subject” include males and females.

As used herein, the term “excipient” means any pharmaceutically acceptable additive, carrier, diluent, adjuvant, or other ingredient, other than the active pharmaceutical ingredient (API), suitably selected with respect to the intended form of administration, and consistent with conventional pharmaceutical practices.

The compounds of the disclosure can be administered alone or as part of a pharmaceutically acceptable composition or formulation. In addition, the compounds can be administered all at once, as for example, by a bolus injection, multiple times, e.g. by a series of tablets, or delivered substantially uniformly over a period of time, as for example, using transdermal delivery. It is also noted that the dose of the compound can be varied over time.

The compounds disclosed herein and other pharmaceutically active compounds, if desired, can be administered to a subject or patient by any suitable route, e.g. orally, topically, rectally, parenterally, (for example, subcutaneous injections, intravenous, intramuscular, intrasternal, and intrathecal injection or infusion techniques), or as a buccal, inhalation, or nasal spray. The administration can be to provide a systemic effect (e.g. enteral or parenteral). All methods that can be used by those skilled in the art to administer a pharmaceutically active agent are contemplated. In some cases, the disclosed formulations can be administered orally or topically.

Suitable oral compositions or formulations in accordance with the disclosure include without limitation tablets, troches, lozenges, aqueous or oily suspensions, dispersible powders or granules, emulsion, hard or soft capsules, syrups or elixirs. Compositions or formulations suitable for oral use may be prepared according to any method known to the art for the manufacture of pharmaceutical compositions.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in microencapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

The pharmaceutical compositions and formulations described herein may also be administered topically or transdermally, especially when the target of treatment includes areas or organs readily accessible by topical application, including diseases of the eye, the skin, or the lower intestinal tract. Suitable topical formulations are readily prepared for each of these areas or organs. Topical application for the lower intestinal tract, e.g., can be effected in a rectal suppository formulation or in a suitable enema formulation. Dosage forms for topical or transdermal administration of a compound described herein include ointments, pastes, creams, lotions, gels, powders, solutions, sprays, inhalants, suppositories, or patches.

For topical applications, the pharmaceutical compositions may be formulated in a suitable ointment, cream, lotion, or gel, containing the active component suspended or dissolved in one or more carriers, and any needed preservatives or buffers as may be required. Carriers for topical administration of the compounds of this disclosure include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2 octyldodecanol, benzyl alcohol and water.

Ophthalmic formulation, eardrops, and eye drops are also contemplated as being within the scope of this disclosure. Additionally, the present disclosure contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound described herein, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are specifically suppositories which can be prepared by mixing the compounds described herein with suitable non-irritating excipients or carriers such as cocoa butter, polyethylene glycol or a suppository wax which are solid at ambient temperature but liquid at body temperature and therefore melt in the rectum or vaginal cavity and release the active compound.

Sterile injectable forms of the compositions described herein may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

The pharmaceutical compositions may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The compounds for use in the methods of the disclosure can be formulated in unit dosage form. The term “unit dosage form” refers to physically discrete units suitable as unitary dosage for subjects undergoing treatment, with each unit containing a predetermined quantity of active material calculated to produce the desired therapeutic effect, optionally in association with a suitable pharmaceutical carrier. The unit dosage form can be for a single daily dose or one of multiple daily doses (e.g., about 1 to 4 or more times per day). When multiple daily doses are used, the unit dosage form can be the same or different for each dose.

The compounds of the disclosure can be administered to a subject or patient at dosage levels in the range of about 0.1 to about 3,000 mg per day. For a normal adult human having a body weight of about 70 kg, a dosage in the range of about 0.01 to about 100 mg per kilogram body weight is typically sufficient. The specific dosage and dosage range that will be used can potentially depend on a number of factors, including the requirements of the subject or patient, the severity of the condition or disease being treated, and the pharmacological activity of the compound being administered. The determination of dosage ranges and optimal dosages for a particular subject or patient is within the ordinary skill in the art.

### Methods of Treatment

The compounds disclosed herein can modulate the aryl hydrocarbon receptor (AHR). AHR is a ligand-activated transcription factor that has been implicated in a variety of conditions, including by modulating the immune system during steady state and during infection and inflammation. The AHR pathway has been recognized for its role in the pathogenesis of diseases and disorders including cancer and viral infections.

Many viral infections have also been found to involve AHR signaling. AHR is implicated in regulating the immune response, and AHR activation has been associated with adaptive immune response impairment and poor health outcomes in a variety of viral infections (e.g., influenza). Inhibition of AHR has also been associated with diminished production of viral particles in vivo (e.g., Dengue fever). AHR appears to play a role in controlling lipid biogenesis, a hallmark of Hepatitis C (HCV) infection. The role of AHR in the regulation of the host immune response against many viruses make modulation of AHR an attractive therapeutic target for viral infections.

Thus, the disclosure provides a method of modulating the aryl hydrocarbon receptor (AHR) in a cell comprising contacting the cell with a therapeutically effective amount of a compound or salt disclosed herein, or a formulation thereof, in an amount effective to modulate the AHR. In some cases, the contacting occurs in vitro. In some cases, the contacting occurs in vivo. In some cases, the contacting comprises administering to a subject in need thereof (e.g., suffers from a disease or disorder associated with aberrant AHR activity). As used herein, the terms “patient” and “subject” may be used interchangeably and mean animals, such as dogs, cats, cows, horses, and sheep (i.e., non-human animals) and humans. Particular patients are mammals (e.g., humans).

Another aspect of the disclosure provides a method of treating a disease or disorder in a subject, comprising administering to the subject a therapeutically effective amount of a compound or salt disclosed herein, or a composition thereof. In some cases, the terms “treating”, “treat” or “treatment” and the like can include preventative (e.g., prophylactic) and palliative treatment.

Diseases or disorders associated with aberrant AHR activity include inflammatory diseases or disorders, cancer, and viral infections. They also include a disease or disorder of the gastrointestinal tract, skin, lung, central nervous system, pancreas, eye, bones bone joints, a neuroinflammatory disease, or a neurodegenerative disease. In some cases, the disease or disorder of the gastrointestinal tract is selected from the group consisting of colitis, inflammatory bowel disease, Crohn's disease, celiac disease, necrotizing enterocolitis, irritable bowel syndrome, chronic idiopathic constipation, traveler's diarrhea, and colorectal cancer. In various cases, the disease or disorder of the skin is selected from the group consisting of atopic dermatitis, acne, psoriasis, and vitiligo. In some cases, the disease or disorder of the eye is abnormal eye movements, inflammatory ocular disease, autoimmune ocular disease, hereditary ocular disease, degenerative ocular disease, vascularization ocular disease, dry and wet age-related macular degeneration (“AMD”), Uveitis, retinitis pigmentosa (“RP”), primary open-angle glaucoma (“POAG”), primary congenital glaucoma, Behcet's disease, or Leber congenital amaurosis (“LCA”). In various cases, the disease or disorder of the lung is lung fibrosis, asthma or chronic obstructive pulmonary disease. In some cases, the disease or disorder of the bone joints is osteoporosis, rheumatoid arthritis, or bone cancer. In various cases, the disease or disorder is diabetes, cancer, a viral infection, or a bacterial infection. In some cases, the bacterial infection is a pulmonary infection, gastrointestinal infection, skin infection, ear infection or a septicemia.

In some cases, the disease or disorder is cancer. In some cases, a cancer to be treated using the compounds and methods described herein include, but are not limited to, a hematological cancer, a lymphoma, a myeloma, a leukemia, a neurological cancer, skin cancer, breast cancer, a prostate cancer, a cancer of the respiratory tract, a cancer of the reproductive organs, a cancer of the digestive tract, a colorectal cancer, lung cancer, head and neck cancer, a gastrointestinal cancer, a liver cancer, a pancreatic cancer, a genitourinary cancer, a bone cancer, renal cancer, and a vascular cancer. In some cases, the cancer is cancer is a liquid or solid tumor, hematological cancer, lymphoma, myeloma, leukemia, sarcoma, eye cancer, thyroid cancer, parathyroid cancer, neurological cancer, skin cancer, breast cancer, endocrine, uterine cancer, endometrial cancer, prostate cancer, colorectal cancer, lung cancer, head and neck cancer, gastrointestinal cancer, liver cancer, pancreatic cancer, genitourinary cancer, bone cancer, renal cancer, or vascular cancer. In some cases, the cancer is bone cancer. Contemplated bone cancers include, but are not limited to, Histiocytoma of Bone. In some cases, the cancer is endocrine cancer. Contemplated endocrine cancers include but are not limited to Multiple Endocrine Neoplasia Syndrome, adrenal cancer (such as without limitation adrenocortical cancer, adrenocortical carcinoma, or adrenocortical adenoma), thyroid cancer (such as without limitation anaplastic thyroid cancer), and parathyroid cancer. In some cases, the cancer is breast cancer. Contemplated breast cancers include, but are not limited to, triple negative breast cancer, invasive ductal carcinoma, invasive lobular carcinoma, ductal carcinoma in situ, and lobular carcinoma in situ. In some cases, the cancer is a cancer of the respiratory tract. Contemplated cancers of the respiratory tract include, but are not limited to, bronchogenic carcinoma, lung carcinoma (such as without limitation small-cell and non-small-cell lung carcinoma), as well as bronchial adenoma and pleuropulmonary blastoma. In some cases, the cancer is central nervous system cancer. Contemplated central nervous system cancers include, but are not limited to, spinal axis tumors, neurofibromatosis-1 associated malignant peripheral nerve sheath tumors (MPNST), and brain cancer. In some cases, the cancer is brain cancer. Contemplated brain cancers include, but are not limited to, glioma, low-grade glioma, astrocytoma (e.g. Grade I—Pilocytic Astrocytoma, Grade II—Low-grade Astrocytoma, Grade III—Anaplastic Astrocytoma, or Grade IV—Glioblastoma (GBM)), glioblastoma multiforme (GBM, also known as glioblastoma), medulloblastoma, craniopharyngioma, ependymoma, Pineal Parenchymal Tumors of Intermediate Differentiation, pinealoma, hemangioblastoma, acoustic neuroma, oligodendroglioma, meningioma, melanoma, neuroblastoma, Esthesioneuroblastoma, or retinoblastoma, medullary carcinoma, brain stem and hypophtalmic glioma, cerebellar and cerebral astrocytoma, as well as neuroectodermal and pineal tumor, chordoma, mixed glioma, optic nerve glioma, subependymoma, metastatic brain tumor, pituitary tumors, pituitary adenoma, primitive neuroectodermal (PNET) tumor, or schwannoma. In some cases, the cancer is a type found more commonly in children than adults, such as brain stem glioma, juvenile pilocytic astrocytoma, optic nerve glioma, pineal tumor, primitive neuroectodermal tumors (PNET), or rhabdoid tumor. In some cases, the patient is an adult human. In some cases, the patient is a child or pediatric patient. In some cases, the cancer is a cancer of the male reproductive organs. Contemplated cancers of the male reproductive organs include, but are not limited to, embryonal carcinoma, seminoma, penile cancer, prostate cancer, and testicular cancer. In some cases, the cancer is a cancer of the female reproductive system. Contemplated cancers of the female reproductive organs include, but are not limited to, ovarian epithelial cancer, fallopian tube cancer, uterine papillary serous carcinoma (UPSC), choriocarcinoma, endometrial, cervical, ovarian, vaginal, and vulvar cancer, as well as sarcoma of the uterus. In some cases, the cancer is an ovarian cancer. Contemplated ovarian cancers include, but are not limited to serous tumor, papillary serous cystadenocarcinoma, endometrioid tumor, mucinous cystadenocarcinoma, granulosa cell tumor, Sertoli-Leydig cell tumor and arrhenoblastoma. In some cases, the cancer is cervical cancer. Contemplated cervical cancers include, but are not limited to squamous cell carcinoma, adenocarcinoma, adenosquamous carcinoma, small cell carcinoma, neuroendocrine tumor, glassy cell carcinoma and villoglandular adenocarcinoma. In some cases, the cancer is a cancer of the digestive tract. Contemplated cancers of the digestive tract include, but are not limited to, anal, colon, colon carcinoma, colorectal, esophageal, stomach, gallbladder, gastrointestinal, Gastrointestinal Carcinoid Tumor, Gastrointestinal Stromal Tumors (GIST), duodenal, gastric, pancreatic, rectal, small-intestine, and salivary gland cancers. In some cases, the cancer is esophageal cancer. Contemplated esophageal cancer include, but are not limited to esophageal cell carcinomas and adenocarcinomas, as well as squamous cell carcinomas, leiomyosarcoma, malignant melanoma, rhabdomyosarcoma and lymphoma. In some cases, the cancer is gastric cancer. Contemplated gastric cancers include, but are not limited to intestinal type and diffuse type gastric adenocarcinoma. In some cases, the cancer is pancreatic cancer. Contemplated pancreatic cancers include, but are not limited to pancreatic ductal carcinoma, pancreatic adenocarcinoma, ductal adenocarcinoma, adenosquamous carcinomas and pancreatic endocrine tumors. In some cases, the cancer is cancer of the urinary tract. Contemplated cancers of the urinary tract include, but are not limited to, bladder, penile, kidney, renal pelvis, ureter, urethral and human papillary renal cancers. In some cases, the cancer is a kidney cancer. Contemplated kidney cancers include, but are not limited to renal cell carcinoma, carcinoma of the renal pelvis, urothelial cell carcinoma, juxtaglomerular cell tumor (reninoma), angiomyolipoma, renal oncocytoma, Bellini duct carcinoma, clear-cell sarcoma of the kidney, mesoblastic nephroma and Wilms' tumor. In some cases, the cancer is bladder cancer. Contemplated bladder cancers include, but are not limited to bladder carcinoma, transitional cell carcinoma, squamous cell carcinoma, adenocarcinoma, sarcoma and small cell carcinoma. In some cases, the cancer is eye cancer. Contemplated eye cancers include, but are not limited to, intraocular melanoma and retinoblastoma. In some cases, the cancer is liver cancer. Contemplated liver cancers include, but are not limited to, cystadenoma, hepatoma, bile duct carcinoma, Extrahepatic Bile Duct Cancer, hepatobilliary (hepatic and billiary duct) cancer, hepatoblastoma, hepatocellular carcinoma (liver cell carcinomas with or without fibrolamellar variant), cholangiocarcinoma (intrahepatic bile duct carcinoma), hepatocholangiocarcinoma and mixed hepatocellular cholangiocarcinoma. In some cases, the cancer is skin cancer. Contemplated skin cancers include, but are not limited to, basal cell carcinoma, sweat gland carcinoma, sebaceous gland carcinoma, squamous cell carcinoma, Kaposi's sarcoma, malignant melanoma, Merkel cell skin cancer, and non-melanoma skin cancer. In some cases, the cancer is head-and-neck cancer. Contemplated head-and-neck cancers include, but are not limited to, squamous cell cancer of the head and neck, laryngeal, hypopharyngeal, nasopharyngeal, oropharyngeal cancer, salivary gland cancer, lip and oral cavity cancer and squamous cell. In some cases, the cancer is lymphoma. Contemplated lymphomas include, but are not limited to, lymphocytic lymphoma, AIDS-related lymphoma, non-Hodgkin's lymphoma, cutaneous T-cell lymphoma, Burkitt lymphoma, Hodgkin's disease, and lymphoma of the central nervous system. In some cases, the cancer is sarcoma. Contemplated sarcomas include, but are not limited to, sarcoma of the soft tissue, fibrosarcoma, neurofibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, malignant fibrous histiocytoma, lymphosarcoma, and rhabdomyosarcoma. In some cases, the cancer is leukemia. Contemplated leukemias include, but are not limited to, acute leukemia (such as without limitation acute myeloid leukemia, acute lymphocytic leukemia, acute lymphoblastic leukemia, chronic lymphocytic leukemia, acute myelocytic leukemia, acute myeloblastic leukemia, acute promyelocytic leukemia, acute myelomonocytic leukemia, acute monocytic leukemia, and acute erythroleukemia), chronic leukemia (such as without limitation chronic myelocytic leukemia and chronic myelogenous leukemia), and hairy cell leukemia.

Other cancers contemplated for treatment with the compounds of the disclosure and methods described herein can be selected from, e.g., urothelial carcinomas, including, but not limited to, bladder cancer and all transitional cell carcinomas; head and neck squamous cell carcinoma; melanoma, including, but not limited to, uveal melanoma; ovarian cancer, including, but not limited to, a serous subtype of ovarian cancer; renal cell carcinoma, including, but not limited to, clear cell renal cell carcinoma subtype; cervical cancer; gastrointestinal/stomach (GIST) cancer, including but not limited to, stomach cancer; non-small cell lung cancer (NSCLC); acute myeloid leukemia (AML); and esophageal cancers. In some cases, the cancer is a urothelial carcinoma. In some cases, the cancer is bladder cancer. In some cases, the cancer is a transitional cell carcinoma. In some cases, the cancer is head and neck squamous cell carcinoma. In some cases, the cancer is a melanoma. In some cases, the cancer is a uveal melanoma. In some cases, the cancer is ovarian cancer. In some cases, the cancer is a serous subtype of ovarian cancer. In some cases, the cancer is renal cell carcinoma. In some cases, the cancer is a clear cell renal cell carcinoma subtype. In some cases, the cancer is cervical cancer. In some cases, the cancer is a gastrointestinal/stomach (GIST) cancer. In some cases, the cancer is a stomach cancer. In some cases, the cancer is non-small cell lung cancer (NSCLC). In some cases, the cancer is advanced and/or metastatic NSCLC. In some cases, the cancer is an esophageal cancer.

Other contemplated cancers include without limitation polycythemia vera, Waldenstrom's macroglobulinemia, multiple myeloma, heavy chain disease, epithelial carcinoma, Appendix Cancer, Atypical Teratoid/Rhabdoid Tumor, Central Nervous System Atypical Teratoid/Rhabdoid Tumor, Central Nervous System Embryonal Tumors, Bronchial Tumors, Carcinoid Tumor, Carcinoma of Unknown Primary, Central Nervous System Cancer, Childhood Cancers, Chordoma, Chronic Myeloproliferative Disorders, Ductal Carcinoma In Situ (DCIS), Embryonal Tumors, Endometrial Cancer, Ependymoblastoma, Extracranial Germ Cell Tumor, Extragonadal Germ Cell Tumor, Eye Cancer, FibrousGerm Cell Tumor, Gestational Trophoblastic Tumor, Heart Cancer, Histiocytosis, Langerhans Cell Cancer, Hypopharyngeal Cancer, Islet Cell Tumors, Langerhans Cell Histiocytosis, Laryngeal Cancer, Lip and Oral Cavity Cancer, Lobular Carcinoma In Situ (LCIS), Macroglobulinemia, Medulloepithelioma, Merkel Cell Carcinoma, Midline Tract Carcinoma Involving NUT Gene, Mouth Cancer, Multiple Endocrine Neoplasia Syndrome, Plasma Cell Neoplasm, Mycosis Fungoides, Myelodysplastic Syndrome, Myelodysplastic/Myeloproliferative Neoplasm, Myeloma, Chronic Myeloproliferative Disorder, Nasal Cavity Cancer, Paranasal Sinus Cancer, Nasopharyngeal Cancer, Oral Cancer, Oral Cavity Cancer, Lip Cancer, Oropharyngeal Cancer, Papillomatosis, Paraganglioma, Paranasal Sinus Cancer, Nasal Cavity Cancer, Pharyngeal Cancer, Pheochromocytoma, Pineoblastoma, Plasma Cell Neoplasm, Pleuropulmonary Blastoma, Transitional Cell Cancer, Sezary Syndrome, Supratentorial Primitive Neuroectodermal Tumors, Throat Cancer, Thymoma, Thymic Carcinoma, Transitional Cell Gestational Trophoblastic Tumor, Unknown Primary, Unusual Cancer of Childhood, or diffuse large B-cell lymphoma (DLBCL).

Also provided are methods and compositions for the diagnosis, prognosis and treatment of viral-associated cancers, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells; as well as virus-associated tumors in gastric cancer, nasopharyngeal carcinoma, cervical cancer, vaginal cancer, vulvar cancer, squamous cell carcinoma of the head and neck, and Merkel cell carcinoma.

In some cases, the compounds of the disclosure, or a composition thereof are useful for the treatment of a viral infection. In some cases, the viral infection to be treated using the compounds and methods described herein include, but are not limited to, coronavirus infections and flavivirus infections.

In some cases, the viral infection is a coronavirus infection. The term “coronavirus infection” as used herein means a disease caused by an infection with a coronavirus. Coronaviruses are a family of viruses that cause diseases in mammals and birds. Coronaviruses are in the subfamily Orthocoronavirinae in the family Coronaviridae, in the order Nidovirales. There are four main genera of coronaviruses, known as alpha, beta, gamma, and delta. Coronaviruses that affect humans include Human coronavirus 229E (HCoV-229E), Human coronavirus OC43 (HCoV—OC43), Severe acute respiratory syndrome-related coronavirus (SARS-CoV), Human coronavirus NL63 (HCoV-NL63, New Haven coronavirus), Human coronavirus HKU1, Middle East respiratory syndrome-related coronavirus (MERS-CoV, previously known as novel coronavirus 2012 and HCoV-EMC), and SARS-CoV-2 (also known as 2019-nCoV).

In some cases, the viral infection is a flavivirus infection. The term “flavivirus infection” as used herein means the disease caused by an infection with a flavivirus.

Flaviviruses are a family of viruses that cause diseases in mammals and insects. Flaviviruses are in the family Flaviviridae, in the order Amarillovirales. Flaviviruses have positive-sense, single-stranded RNA genomes which are non-segmented and around 10-11 kbp in length. In humans, flaviviruses cause hemorrhagic fever, encephalitis, and the birth defect microcephaly. Flavivirus infections range from being asymptomatic to causing death. There are vaccines or antiviral drugs to prevent or treat some, but not all, human flavivirus infections. Non-limiting examples of flaviviruses include West Nile virus, dengue virus, tick-borne encephalitis virus, yellow fever virus, Zika virus and several other viruses which may cause encephalitis. Thus, in some cases, the compounds of the disclosure (such as a compound of Formula (I), a compound of Table 1, and salts thereof), or pharmaceutically acceptable salt thereof can be used to treat flavivirus infections, such as West Nile fever, dengue fever, tick-borne encephalitis, yellow fever, and Zika fever (i.e., West Nile virus infection, dengue virus infection, tick-borne encephalitis virus infection, yellow fever virus infection, and Zika virus infection, respectively).

Another aspect of the disclosure provides the use of a compound or salt disclosed herein or a composition comprising a compound or salt disclosed herein in the treatment of a disease or disorder associated with aberrant AHR activity.

Also contemplated is the use of a compound or salt disclosed herein, or a composition comprising a compound or salt disclosed herein in the manufacture of a medicament in the treatment of a disease or disorder associated with aberrant AHR activity.

Use of a compound as disclosed herein, or a pharmaceutically acceptable salt thereof to treat a disease or disorder associated with aberrant AHR activity in a subject also is contemplated. Further, the use of a compound as disclosed herein, or a pharmaceutically acceptable salt thereof, also is contemplated. Furthermore, use of a compound as disclosed herein, or a pharmaceutically acceptable salt in the preparation of a medicament for use in treating the aforementioned conditions also are contemplated.

In some cases, the compound as disclosed herein, or pharmaceutically acceptable salt thereof, can be administered in combination with another therapeutic agent to treat a disease or disorder associated with aberrant AHR activity. Thus, in any of the methods disclosed herein, treatment of a disease or disorder can include co-administration of the compound of the disclosure, or a pharmaceutically acceptable salt thereof, in combination with another therapeutic agent. In some cases, the therapeutic agent is an anti-inflammatory agent. Contemplated anti-inflammatory agents include, but are not limited to, mesalazine, naproxen, ibuprofen, diclofenac, celecoxib, sulindac, oxaprozin, piroxicam, indomethacin, meloxicam, fenoprofen, difunisal, etodolac, ketorolac tromethamine, meclofenamate, nabumetone, salsalate, or any combination of the foregoing. In some cases, the therapeutic agent is an immune checkpoint inhibitor. Non-limiting examples of immune checkpoint inhibitors include PD1 or PDL1 antibodies, such as pembrolizumab, nivolumad, cemiplimab, atezolizumab, dostarlimab, durvalumab, or avelumab.

### Synthesis of the Compounds of the Disclosure

The compounds of the disclosure can be synthesized by any method known in the art. For example, the compounds of the disclosure can be synthesized as described in the examples below.

A general scheme for synthesis of compounds of the disclosure is shown below. Further information on particular reaction conditions is provided in detail in the Examples section.

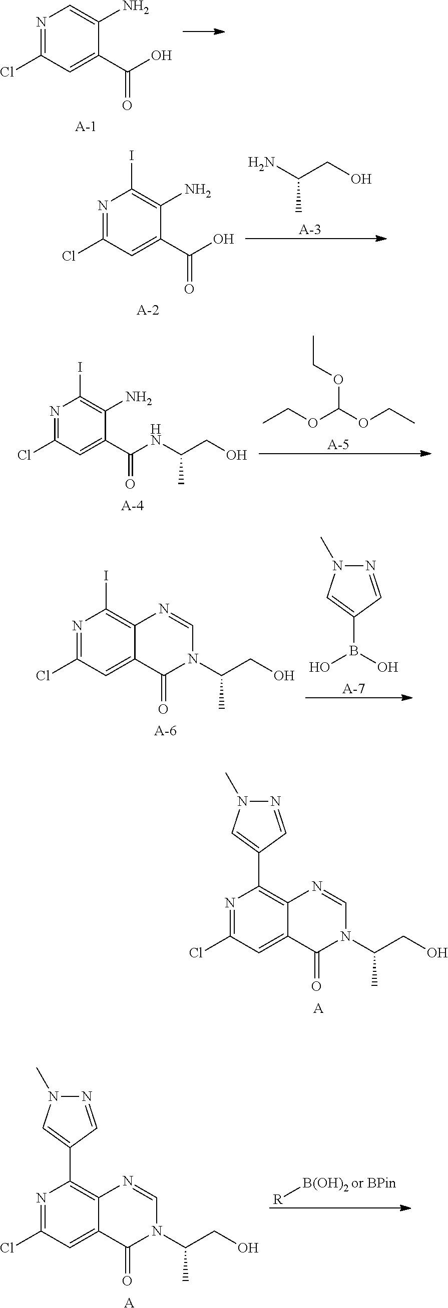

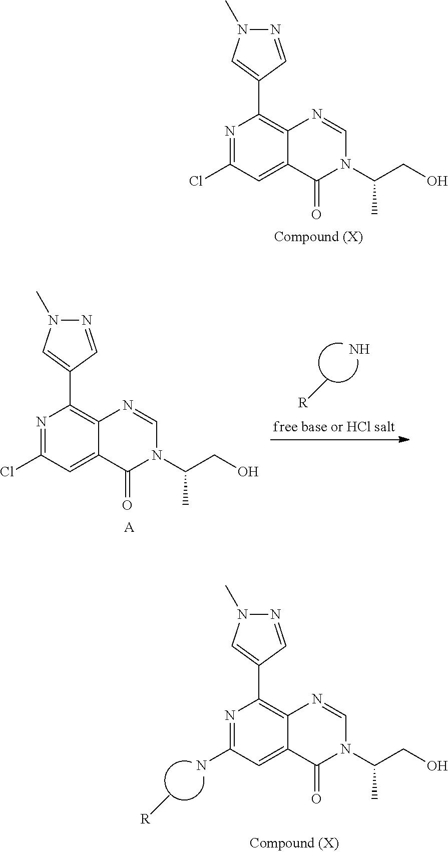

The following synthetic scheme provides additional routes for preparation of compounds of the disclosure.

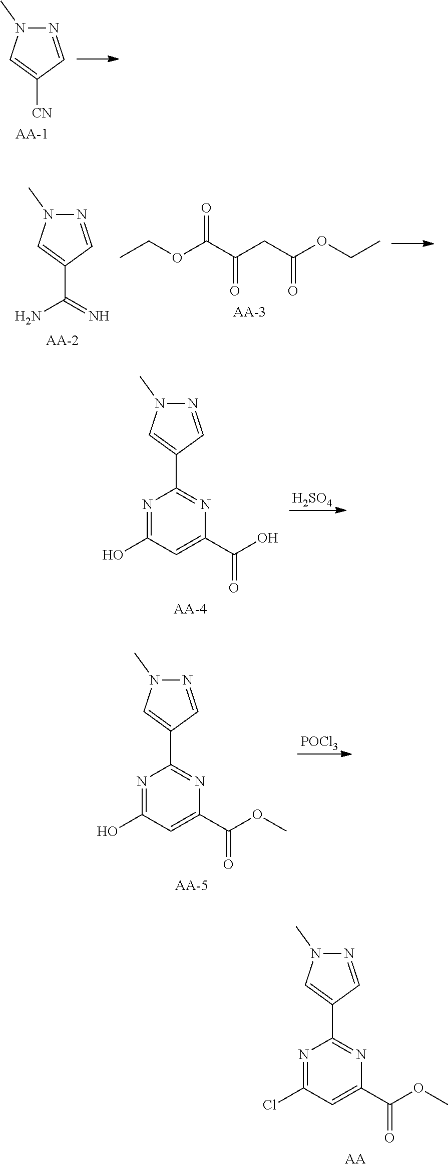

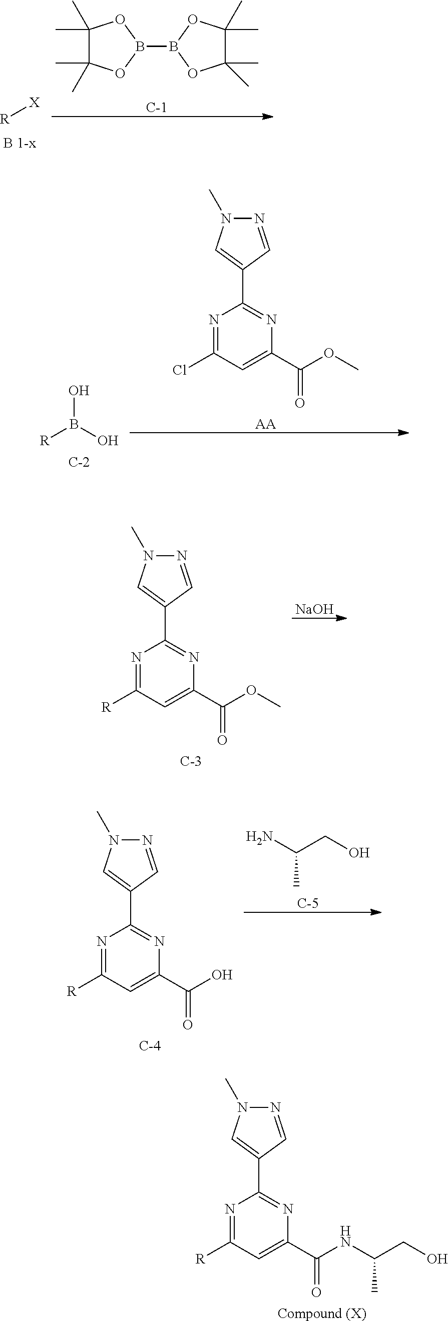

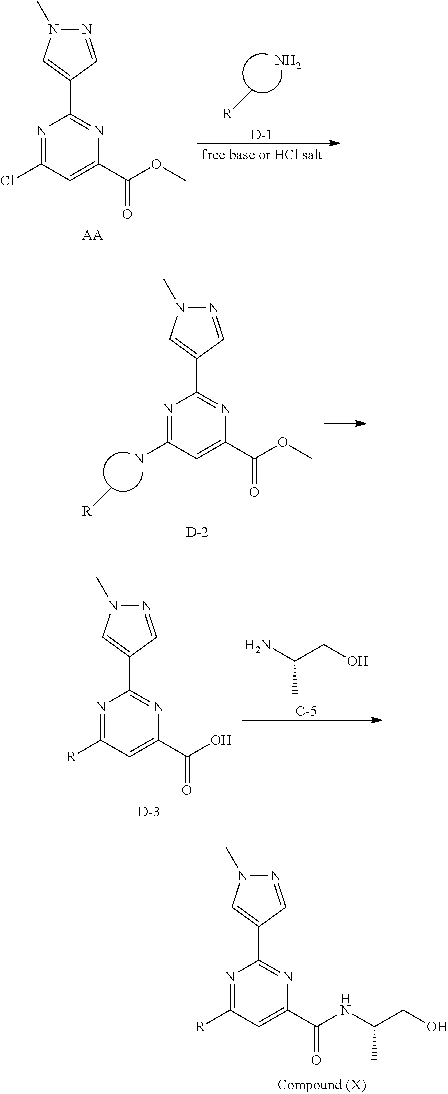

### Embodiments of the Disclosure

1. A compound, or pharmaceutically acceptable salt thereof, having a structure of Formula (I) or Formula (II):

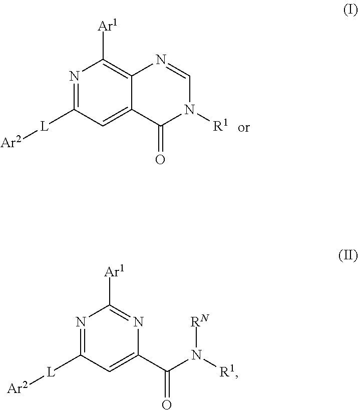

L is a bond, C(O), C₂₋₃alkenylene, or C₂₋₃alkynylene;
Ar¹ is phenyl, C₃₋₈cycloalkyl, or 4-8 membered aromatic or non-aromatic heterocyclyl having 1-3 ring heteroatoms selected from O, N, and S, and Ar¹ is optionally substituted with 1 or 2 R^{A};
each R^{A} is independently halo, OH, ═O, CN, C₁₋₆alkyl, C₁₋₆haloalkyl, C₁₋₆hydroxyalkyl, C₁₋₆alkoxy, C₁₋₆haloalkoxy, cyclopropyl, NR^{N}R^{N}, C(O)NR^{N}R^{N}, CO₂R^{N}, C(O)R^{N}, or SO₂—C₁₋₆alkyl;
each R^{N} is independently H or C₁₋₆alkyl;
Ar² is 5-10 membered aromatic or non-aromatic ring having 0-3 ring heteroatoms selected from O, N, and S, and Ar² is optionally substituted with 1, 2, or 3 R^{B};
each R^{B} is independently halo, CN, C₁₋₆alkyl, OH, ═O, C₁₋₆hydroxyalkyl, C(O)R^{N}, CO₂R^{N}, C(O)NR^{N}R^{N}, Het, C(O)—C₀₋₂alkylene-Het, or N(R^{N})—C₀₋₃alkylene-C(O)—C₀₋₃alkylene-Het;
Het is a 4-8-membered aromatic or non-aromatic ring having 0-3 ring heteroatoms selected from N, O, and S, and is optionally substituted with 1, 2, or 3 R^{C};
each R^{C} is independently halo, OH, ═O, CN, C₁₋₆alkyl, C₁₋₆haloalkyl, C₁₋₆hydroxyalkyl, C₁₋₆alkoxy, C₁₋₆haloalkoxy, NR^{N}R^{N}, C(O)NR^{N}R^{N}, CO₂R^{N}, C(O)R^{N}, or SO₂—C₁₋₆alkyl;
R¹ is C₁₋₆alkyl, C₁₋₆alkylene-R^{D}, C₁₋₆alkylene-C(O)R^{D}, C₁₋₆alkylene-NHSO₂cyclopropyl, or C₁₋₆alkylene-NR^{N}—C(O)R^{N}; and
R^{D} is OR^{N} or NR^{N}R^{N},
with the proviso that
(i) for compounds of Formula (I), when L is a bond, then (a) Ar² is substituted with at least one R^{B} selected from Het, C(O)—C₀₋₂alkylene-Het, and N(R^{N})—C₀₋₃alkylene-C(O)—C₀₋₃alkylene-Het or (b) Ar² comprises a 8-10-membered bicyclic aromatic or non-aromatic ring having 1-3 ring heteroatoms and is substituted with 0-3 R^{B}; and
(ii) for compounds of Formula (II), when L is a bond, Ar² is phenyl or a 6-membered aromatic heterocyclyl, Ar² is substituted with 1 R^{B}, and R^{B} is Het, then (a) Het is attached to Ar² via a carbon ring atom of Het or (b) Het is substituted with 1, 2, or 3 R^{C}.

2. The compound or salt of embodiment 1, wherein each R^{N} is independently H or methyl.

3. The compound or salt of embodiment 1 or 2, wherein R¹ is C₁₋₆alkyl or C₁-6alkylene-R^{D}.

4. The compound or salt of embodiment 3, wherein R^{D} is OR^{N}.

5. The compound or salt of embodiment 3, wherein R¹ is

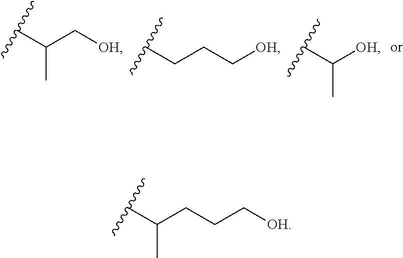

6. The compound or salt of any one of embodiments 1 to 42, wherein R¹ is

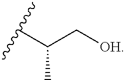 [SMILES: *[C@@H](C)CO]

7. The compound or salt of embodiment 1 or 2, having a structure of Formula (Ia):

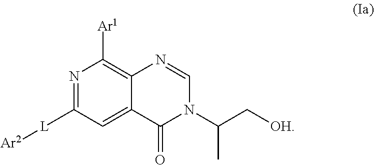

8. The compound or salt of embodiment 1 or 2, having a structure of Formula (IIa):

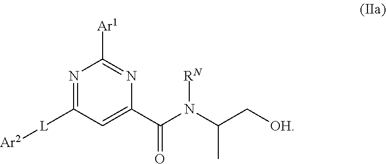

9. The compound or salt of any one of embodiments 1-8, wherein Ar¹ is heterocyclyl comprising 4-8 total ring atoms, wherein 1-3 of the ring atoms are selected from O, N, and S.

10. The compound or salt of any one of embodiments 1-9, wherein Ar¹ is substituted with 1 or 2 R^{A}.

11. The compound or salt of embodiment 9, wherein Ar¹ is unsubstituted.

12. The compound or salt of any one of embodiments 1-11, wherein Ar¹ is phenyl, C₃₋₈cycloalkyl, or pyrazole.

13. The compound or salt of any one of embodiments 1-7, 9, and 10, having a structure of Formula (Ib):

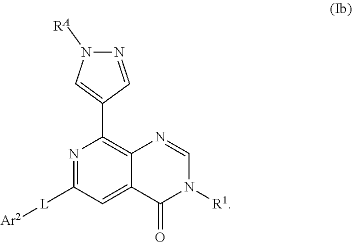

14. The compound or salt of any one of embodiments 1-6, 8, 9, and 10, having a structure of Formula (IIb):

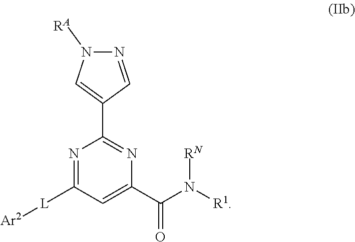

15. The compound or salt of any one of embodiments 9, 10, and 12-14, wherein at least one R^{A} is C₁₋₆alkyl, C₁₋₆haloalkyl, or C₁₋₆alkoxy, or at least one R^{A} is methyl.

16. The compound or salt of embodiment 1 or 2, having a structure of Formula (Ic):

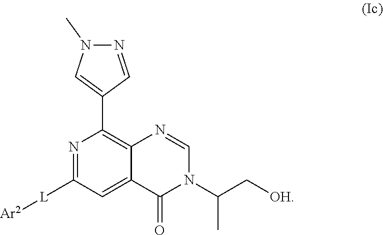

17. The compound or salt of embodiment 1 or 2, having a structure of Formula (IIc):

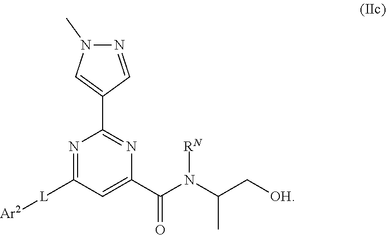

18. The compound or salt of any one of embodiments 1-17, wherein L is a bond.

19. The compound or salt of any one of embodiments 1-17, wherein L is C₂-3alkenylene.

20. The compound or salt of any one of embodiments 1-17, wherein L is C₂-3alkynylene.

21. The compound or salt of any one of embodiments 1-20, wherein Ar² is phenyl or naphthyl.

22. The compound or salt of any one of embodiments 1-20, wherein Ar² is a 5-10 membered aromatic or non-aromatic ring having 1-3 ring heteroatoms selected from O, N, and S.

23. The compound or salt of embodiment 22, wherein Ar² is an 8-10 bicyclic aromatic or non-aromatic ring having 1-3 ring heteroatoms selected from O, N, and S.

24. The compound or salt of embodiment 23, wherein Ar² is isoindoline, tetrahydroisoquinoline, tetrahydroquinoline, indazole, or pyrazolo[4,3-c]pyridine.

25. The compound or salt of any one of embodiments 1-24, wherein Ar² is unsubstituted.

26. The compound or salt of any one of embodiments 1-24, wherein Ar² is substituted with 1, 2, or 3 R^{B}.

27. The compound or salt of embodiment 26, wherein at least one R^{B} is halo, CN, C₁₋₆alkyl, OH, ═O, C₁₋₆hydroxyalkyl, C(O)R^{N}, CO₂R^{N}, or C(O)NR^{N}R^{N}.

28. The compound or salt of embodiment 26 or 27, wherein at least one R^{B} is Het, C(O)—C₀₋₂alkylene-Het, or N(R^{N})—C₀₋₃alkylene-C(O)—C₀₋₃alkylene-Het.

29. The compound or salt of embodiment 28, wherein Het comprises phenyl, pyridine, pyridine-2-one, pyrimidine, piperidine, piperazine, morpholine, azepane, 1,4-diazepane, pyrrolidine, tetrahydropyran, dihydropyran, or 8-oxa-3-azabicyclo[3.2.1]octane.

30. The compound or salt of embodiment 28 or 29, wherein Het is unsubstituted.

31. The compound or salt of embodiment 28 or 29, wherein Het is substituted with 1 or 2 R^{C}.

32. The compound or salt of embodiment 31, wherein at least one R^{C} is halo, OH, ═O, C₁₋₆alkyl, CO₂R^{N}, NR^{N}R^{N}, or SO₂—C₁₋₆alkyl.

33. The compound or salt of embodiment 32, wherein at least one R^{C} is F, Cl, OH, ═O, methyl, CO₂H, NH₂, or SO₂CH₃.

34. The compound or salt of embodiment 28 or 29, wherein Het is

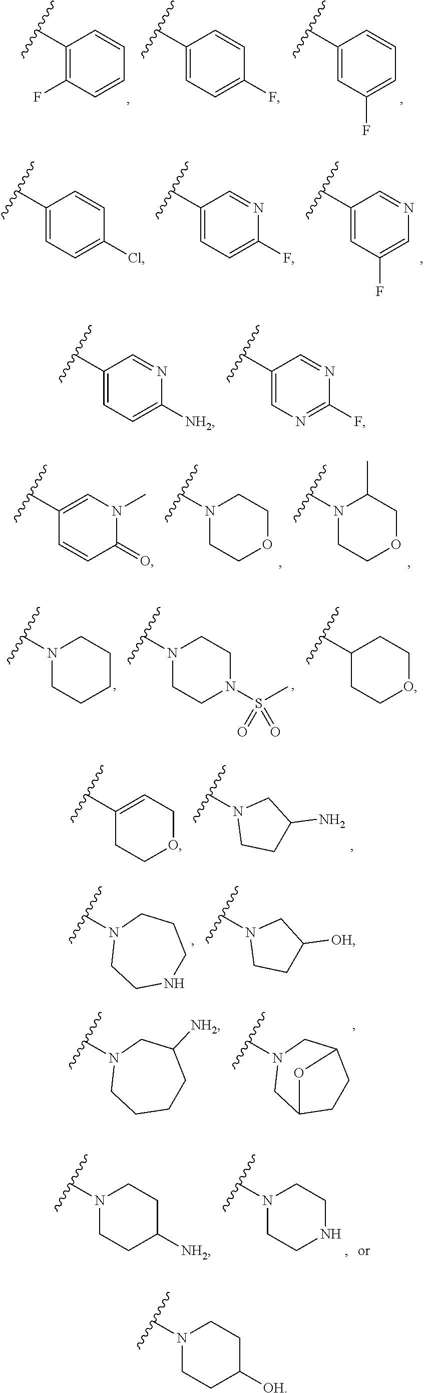

35. The compound or salt of any one of embodiments 1-20, wherein Ar² is

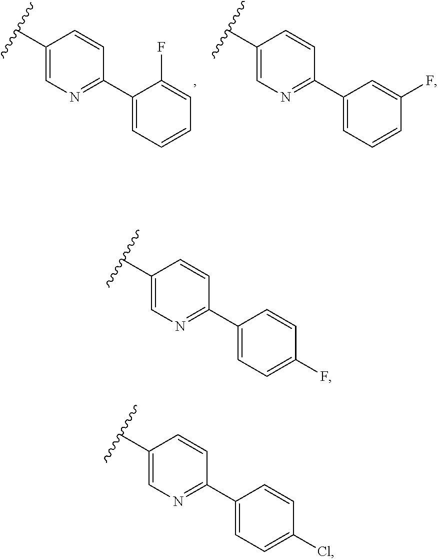 [SMILES: *c1ccc(-c2ccccc2F)nc1 ; *c1ccc(-c2cccc(F)c2)nc1 ; *c1ccc(-c2ccc(F)cc2)nc1 ; *c1ccc(-c2ccc(Cl)cc2)nc1]

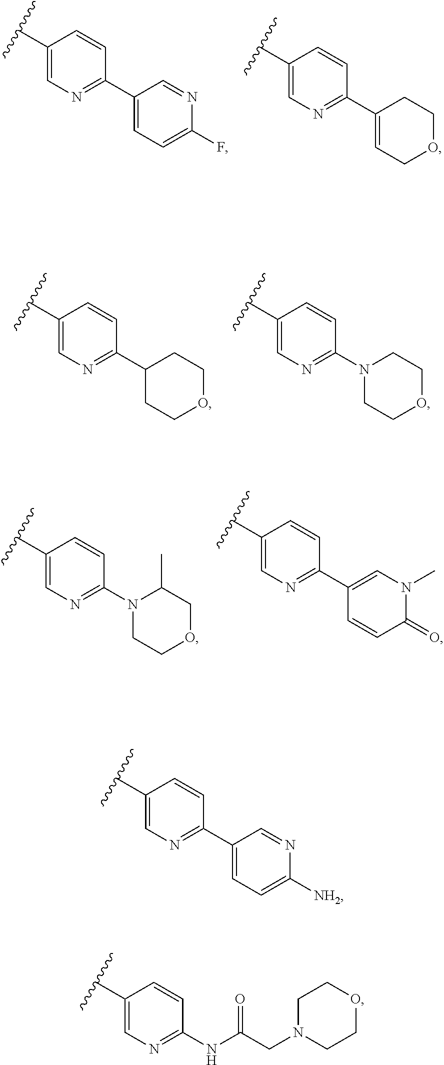

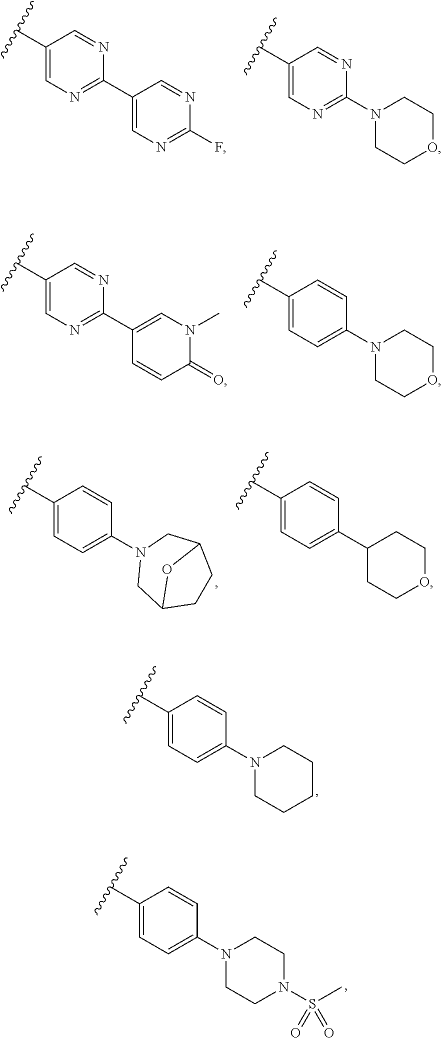

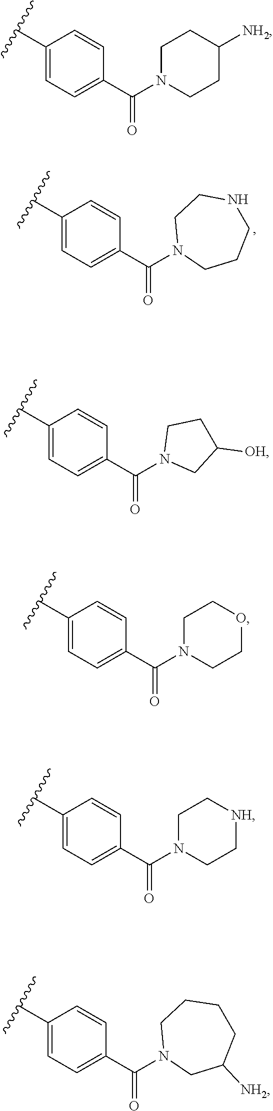 [SMILES: *c1ccc(C(=O)N2CCC(N)CC2)cc1 ; *c1ccc(C(=O)N2CCCNCC2)cc1 ; *c1ccc(C(=O)N2CCC(O)C2)cc1 ; *c1ccc(C(=O)N2CCOCC2)cc1 ; *c1ccc(C(=O)N2CCNCC2)cc1 ; *c1ccc(C(=O)N2CCCCC(N)C2)cc1]

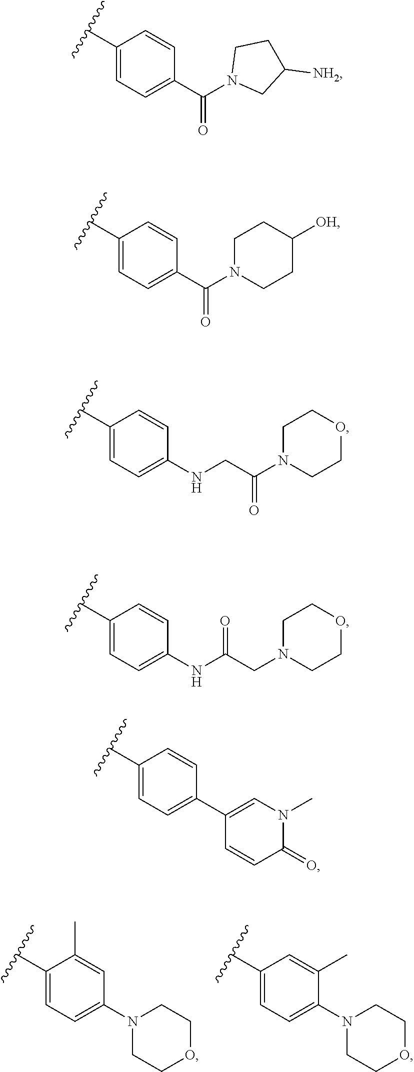

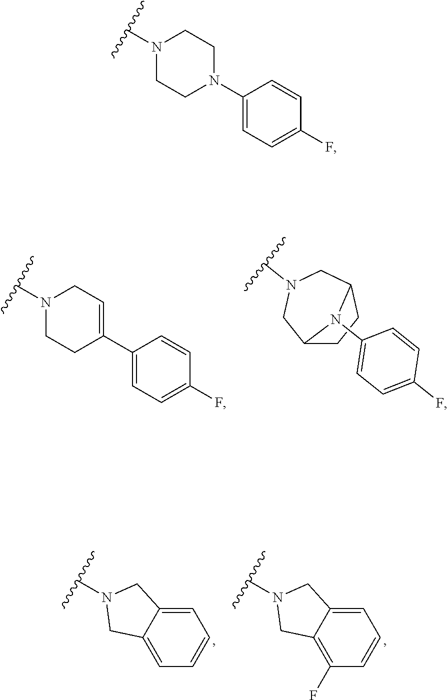 [SMILES: *N1CCN(c2ccc(F)cc2)CC1 ; *N1CC=C(c2ccc(F)cc2)CC1 ; *N1CC2CCC(C1)N2c1ccc(F)cc1 ; *N1Cc2ccccc2C1 ; *N1Cc2cccc(F)c2C1]

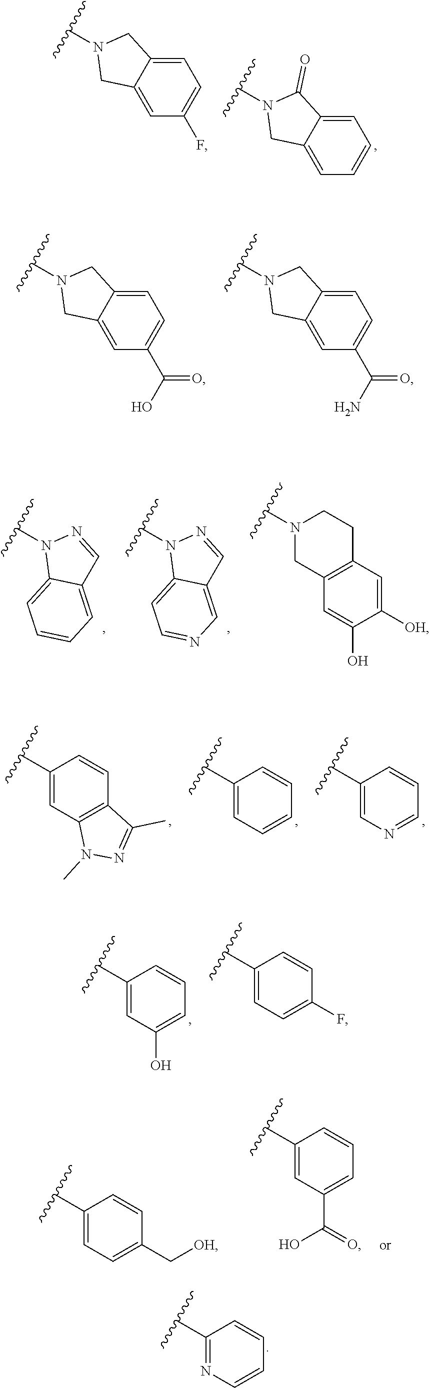

36. The compound or salt of any one of embodiments 1-20, wherein Ar² is

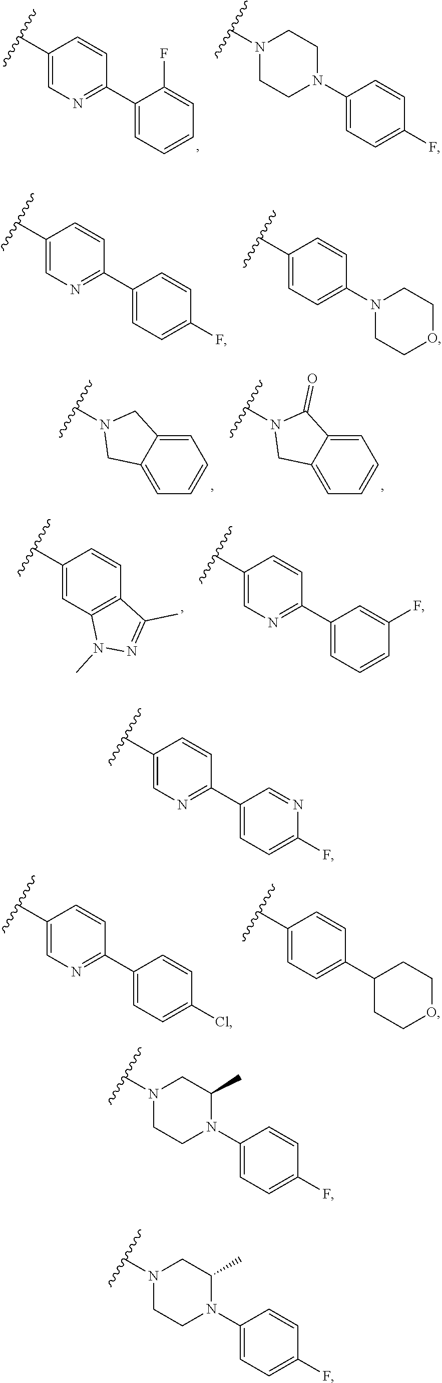

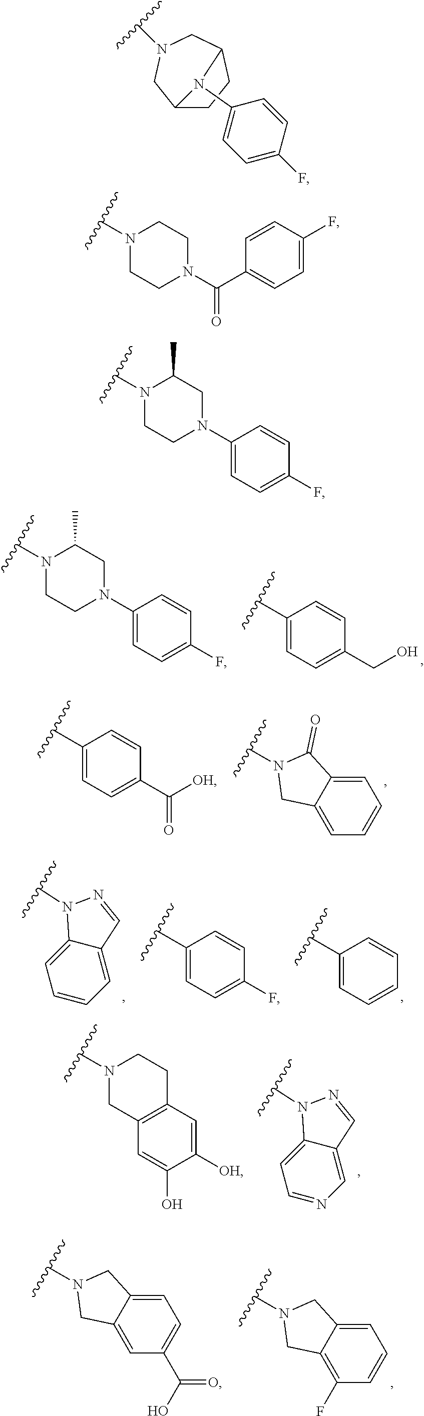

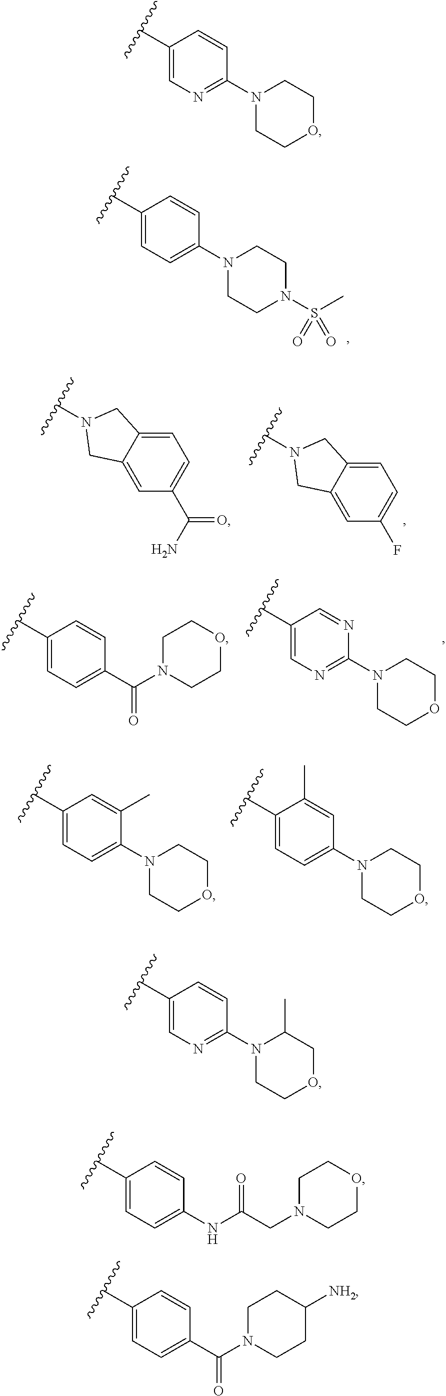

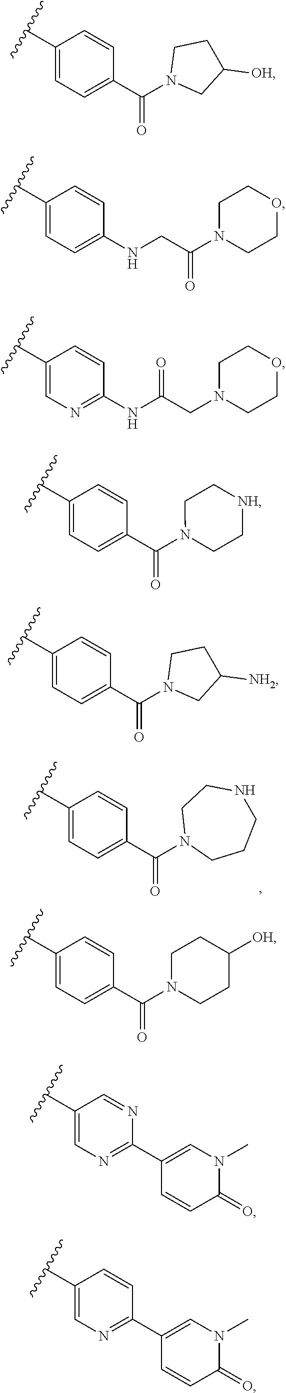

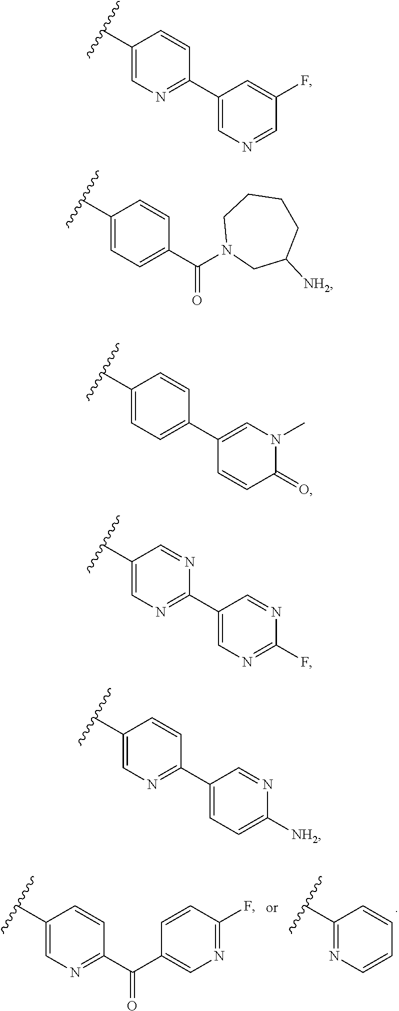

37. A compound as listed in Table 1, or a pharmaceutically acceptable salt thereof.

38. A pharmaceutical formulation comprising the compound or salt of any one of embodiments 1 to 37 and a pharmaceutically acceptable excipient.

39. A method of treating or preventing a disease or disorder associated with aberrant aryl hydrocarbon receptor (AHR) activity in a subject, comprising administering to the subject a therapeutically effective amount of the compound or salt of any one of embodiments 1-37.

40. The method of embodiment 39, wherein the disease or disorder is an inflammatory disease or disorder.

41. The method of embodiment 39, wherein the disease or disorder is a disease or disorder of the gastrointestinal tract, skin, lung, central nervous system, pancreas, eye, bones bone joints, a neuroinflammatory disease, or a neurodegenerative disease.

42. The method of embodiment 41, wherein the disease or disorder of the gastrointestinal tract is selected from the group consisting of colitis, inflammatory bowel disease, Crohn's disease, celiac disease, necrotizing enterocolitis, irritable bowel syndrome, chronic idiopathic constipation, traveler's diarrhea, and colorectal cancer.

43. The method of embodiment 41, wherein the disease or disorder of the skin is selected from the group consisting of atopic dermatitis, acne, psoriasis, and vitiligo.

44. The method of embodiment 41, wherein the disease or disorder of the eye is abnormal eye movements, inflammatory ocular disease, autoimmune ocular disease, hereditary ocular disease, degenerative ocular disease, vascularization ocular disease, dry and wet age-related macular degeneration (“AMD”), Uveitis, retinitis pigmentosa (“RP”), primary open-angle glaucoma (“POAG”), primary congenital glaucoma, Behcet's disease, or Leber congenital amaurosis (“LCA”).

45. The method of embodiment 41, wherein the disease or disorder of the lung is lung fibrosis, asthma or chronic obstructive pulmonary disease.

46. The method of embodiment 41, wherein the disease or disorder of the bone joints is osteoporosis, rheumatoid arthritis, or bone cancer.

47. The method of embodiment 39, wherein the disease or disorder is diabetes, cancer, a viral infection, or a bacterial infection.

48. The method of embodiment 47, wherein the viral infection is flavivirus infection or coronavirus infection.

49. The method of embodiment 47, wherein the bacterial infection is a pulmonary infection, gastrointestinal infection, skin infection, ear infection or a septicemia.

50. The method of any one of embodiments 39-49, further comprising administering to the patient a therapeutic agent.

51. The method of embodiment 50, wherein the therapeutic agent is an anti-inflammatory agent.

52. The method of embodiment 51, wherein the anti-inflammatory agent is selected from mesalazine, naproxen, ibuprofen, diclofenac, celecoxib, sulindac, oxaprozin, piroxicam, indomethacin, meloxicam, fenoprofen, difunisal, etodolac, ketorolac tromethamine, meclofenamate, nabumetone, salsalate, or any combination of the foregoing.

53. The method of embodiment 50, wherein the therapeutic agent is an immune checkpoint inhibitor.

54. A compound or salt of any one of embodiments 1-37 for use in the treatment or prevention of a disease or disorder associated with aberrant aryl hydrocarbon receptor (AHR) activity.

55. Use of the compound or salt of any one of embodiments 1-37 for treating or preventing a disease or disorder associated with aberrant aryl hydrocarbon receptor (AHR) activity.

### EXAMPLES

### Abbreviations

| | |
|---|---|
| ACN | acetonitrile |
| AcOH | acetic acid |
| aq. | aqueous |
| DCM | dichloromethane |
| DIEA | N-ethyl diisopropylamine (Hünig's base) |
| DMF | N,N-dimethylformamide |
| DMSO | dimethyl sulfoxide |
| EDCI | 1-ethyl-3-(3-dimethylaminopropyl)carbodiimide |
| eq or eq. or | equivalent |
| equiv. | |
| ESI or ES | electrospray ionization |
| EtOAc | ethyl acetate |
| EtOH | ethanol |
| g or gr | gram(s) |
| h | hour(s) |
| HATU | 1-[bis(dimethylamino)methylene]-1H-1,2,3- |
| | triazolo[4,5-b]pyridinium 3-oxide hexafluorophosphate |
| HOBT | hydroxybenzotriazole |
| LC-MS | liquid chromatography mass spectroscopy |
| m/z | mass divided by charge |
| M | molar |
| MeOH | methanol |
| mg | milligrams |
| min | minutes |
| mL | milliliters |
| MS | mass spectra |
| NIS | N-iodo-succinimide |
| NMR | nuclear magnetic resonance |
| Pd(dppf)Cl₂ | [1,1′-bis(diphenylphosphino)ferrocene] |
| | dichloropalladium(II) |
| PE | petroleum ether |
| RT or rt or r.t. | room temperature |
| sat. or satd. | saturated |
| TEA | triethylamine |
| temp | temperature |
| THF | tetrahydrofuran |

### Preparation of 3-amino-6-chloro-2-iodoisonicotinic acid (A-2)

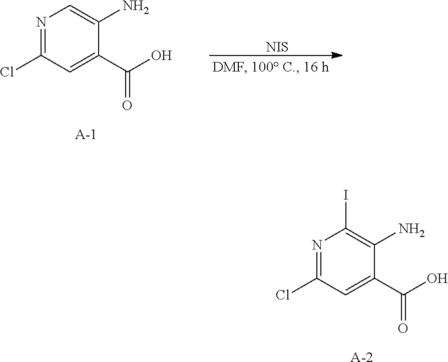 [SMILES: Nc1cnc(Cl)cc1C(=O)O ; "NIS" ; "A-1" ; Nc1c(C(=O)O)cc(Cl)nc1I ; "A-2"]

To a solution of 5-amino-2-chloroisonicotinic acid (1.0 g, 5.82 mmol) in DMF (30 mL) was added NIS (2.6 g, 11.64 mmol). The reaction mixture was stirred at 60° C. for 48 h. The desired mass was detected on LC-MS. The residue was then quenched with water, extracted with EtOAc, the organic layer was washed with brine, dried over Na₂SO₄, filtered and concentrated to afford A-2 (830.0 mg, yield: 48.0%) as a yellow solid. LCMS (ESI): calcd for C₆H₄ClIN₂O₂ [M+H]⁺ m/z 299.4, found 299.0.

### Preparation of (S)-3-amino-6-chloro-N-(1-hydroxypropan-2-yl)-2-iodoisonicotinamide (A-4)

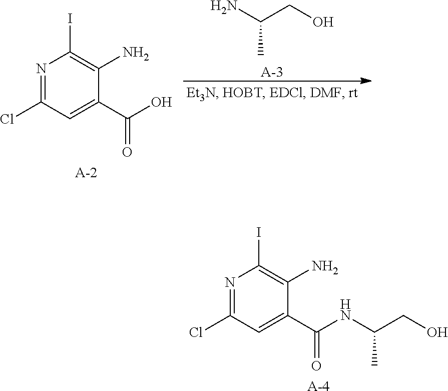

To a solution of A-2 (830.0 mg, 2.78 mmol) in DMF (30 mL) was added EDCI (640.0 mg, 3.33 mmol), HOBT (440.0 mg, 3.33 mmol) and TEA (420.0 mg, 4.17 mmol). After 30 min, A-3 (230.0 mg, 3.05 mmol) was added and the reaction mixture was stirred at 60° C. for 12 h. The desired mass was detected on LC-MS. The residue was extracted with EtOAc (50 mL), the organic layer was washed with brine, dried over Na₂SO₄, filtered and concentrated. The residue was purified by silica gel chromatography (eluting with PE/EtOAc from 100/0 to 50/50 in 30 mins) to afford A-4 (530.0 mg, yield: 53.6%) as a brown solid. LCMS (ESI): calcd for C₉H₁₁ClIN₃O₂ [M+H]⁺ m/z 356.5, found 356.1.

### Preparation of (S)-6-chloro-3-(1-hydroxypropan-2-yl)-8-iodopyrido[3,4-d]pyrimidin-4(3H)-one (A-6)

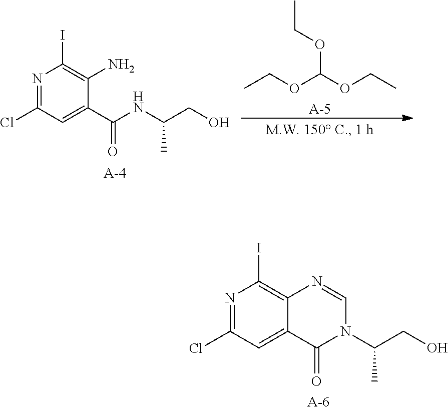

To a solution of A-4 (530.0 mg, 1.49 mmol) in AcOH (50 mL) was added triethoxymethane (330.0 mg, 2.23 mmol). The mixture was heated at 150° C. in a microwave reactor for 1 h under an atmosphere of N₂. The desired mass was detected on LC-MS. The residue mixture was then quenched to ice water, extracted with EtOAc. The organic phases were combined, washed with brine, dried over Na₂SO₄, filtered and concentrated in vacuo. The residue was purified by silica gel chromatography (eluting with PE/EtOAc from 100/0 to 80/20 in 30 mins) to afford A-6 (230.1 mg, yield: 42.2%) as a brown solid. LCMS (ESI): calcd for C₁₀H₉ClIN₃O₂ [M+H]⁺ m/z 366.5, found 366.0.

### Preparation of (S)-6-chloro-3-(1-hydroxypropan-2-yl)-8-(1-methyl-1H-pyrazol-4-yl)pyrido[3,4-d]pyrimidin-4(3H)-one (A)

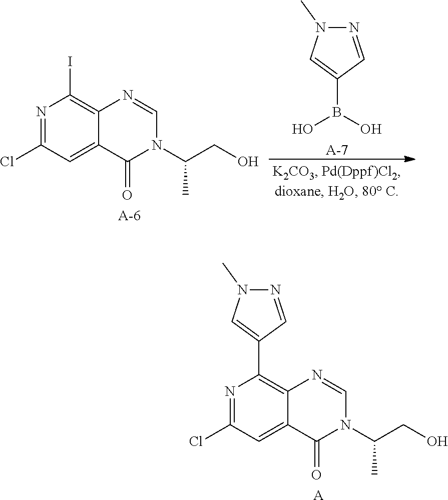

To a solution of A-6 (230.1 mg, 0.86 mmol) in 1,4-dioxane (10 mL) and H₂O (2 mL) was added A-7 (115.3 mg, 0.94 mmol), Pd(dppf)Cl₂ (125.7 mg, 0.17 mmol) and K₂CO₃ (237.7 mg, 1.72 mmol). The mixture was stirred at 80° C. for 12 hours under N₂. The desired mass was detected on LC-MS. The residue was extracted with EtOAc (100 mL×3). The organic phases were combined, washed with brine, dried over Na₂SO₄, filtered and concentrated in vacuo. The residue was purified by silica gel chromatography (eluting with PE/EtOAc form 100/0 to 80/20 in 30 mins) to afford A (124.9 mg, yield: 62.1%) as a brown solid. LCMS (ESI): calcd for C₁₄H₁₄ClN₅O₂ [M+H]⁺ m/z 320.7, found 320.2.

### Preparation of Compounds 1-13

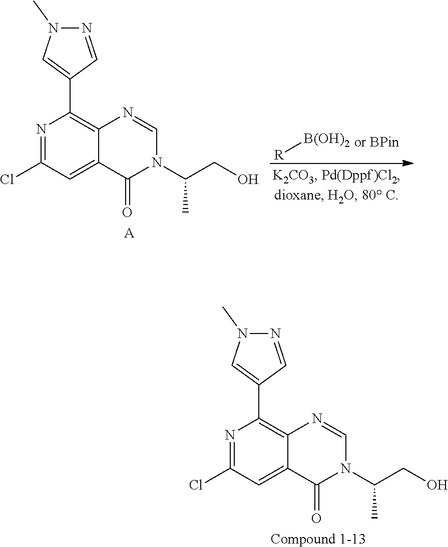

To a solution of intermediate A (1.0 eq) in dioxane (6 mL)/H₂O (2 ml), boric acid or borate ester (1.1 eq), K₂CO₃ (2.0 eq) and Pd(dppf)Cl₂ (0.05 eq) were added. The reaction mixture was stirred at 90° C. for 3 h. The desired mass was detected on LC-MS. The residue was quenched by ice water, extracted with EtOAc. The organic phases were combined, washed with brine, dried over Na₂SO₄, and concentrated in vacuo. The residue was purified by was purified by C18 column (eluting with ACN-H₂O (0.1% NH₃·H₂O) from 5/95 from 80/20 in 30 mins) to afford final compound.

### Preparation of Compounds 14-17

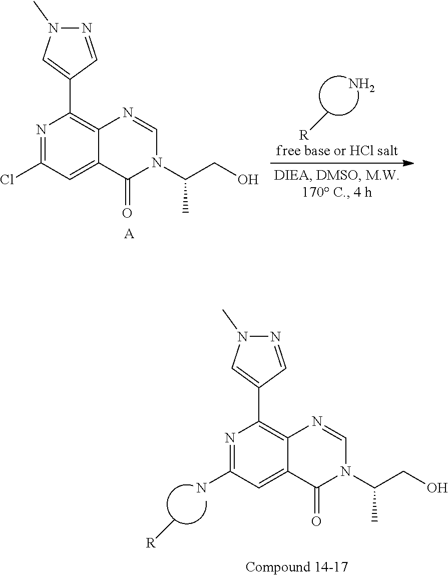

In a vial, intermediate A (1.0 eq), cyclo-amine (1.5 eq) and DIEA (3.0 eq) were dissolved in DMSO (3 ml). The mixture was reacted at 170° C. with microwave for 4 h. The desired mass was detected on LC-MS. The residue was quenched by ice water, extracted with EtOAc. The organic phases were combined, washed with brine, dried over Na₂SO₄, and concentrated in vacuo. The residue was purified by was purified by C₁₈ column (eluting with ACN-H₂O (0.1% NH₃·H₂O) from 5/95 from 80/20 in 30 mins) to afford final compound.

Compounds in the below table were prepared in a manner similar to that described above.

| | LCMS | |
|---|---|---|
| | (ESI): | |
| | [M + H]⁺ | Analytics |
| 1 | 457.3 | ¹H NMR (400 MHz, DMSO-d₆) δ 9.60 (d, J = 2.3 |
| | | Hz, 1H), 8.85 (s, 1H), 8.76 (dd, J = 8.4, 2.4 Hz, |
| | | 1H), 8.57 (s, 1H), 8.55 (s, 1H), 8.43 (s, 1H), 8.07 |
| | | (td, J = 7.9, 1.8 Hz, 1H), 7.96 (dd, J = 8.3, 2.1 Hz, |
| | | 1H), 7.60-7.49 (m, 1H), 7.44-7.34 (m, 2H), |
| | | 5.10 (t, J = 5.6 Hz, 1H), 4.96-4.85 (m, 1H), 3.98 |
| | | (s, 3H), 3.89-3.79 (m, 1H), 3.74-3.65 (m, 1H), |
| | | 1.46 (d, J = 7.1 Hz, 3H). |
| 2 | 388.2 | ¹H NMR (400 MHz, DMSO-d₆) δ 8.79 (s, 1H), |
| | | 8.52 (s, 1H), 8.50 (s, 1H), 7.91-7.83 (m, 2H), |
| | | 7.77-7.72 (m, 2H), 7.55-7.47 (m, 1H), 7.47- |
| | | 7.39 (m, 2H), 7.37-7.31 (m, 1H), 5.08 (t, J = 5.6 |
| | | Hz, 1H), 4.93-4.84 (m, 1H), 3.97 (s, 3H), 3.85- |
| | | 3.76 (m, 1H), 3.72-3.63 (m, 1H), 1.44 (d, J = 7.0 |
| | | Hz, 3H). |
| 3 | 447.3 | ¹H NMR (400 MHz, DMSO-d₆) δ 8.76 (s, 1H), |
| | | 8.50-8.45 (m, 2H), 8.18-8.11 (m, 3H), 7.08 (d, |
| | | J = 8.8 Hz, 2H), 5.13-5.04 (m, 1H), 4.95-4.84 |
| | | (m, 1H), 3.96 (s, 3H), 3.86-3.74 (m, 5H), 3.72- |
| | | 3.65 (m, 1H), 3.23 (t, J = 4.9 Hz, 4H), 1.44 (d, |
| | | J = 7.1 Hz, 3H). |
| 4 | 473.3 | ¹H NMR (400 MHz, DMSO-d₆) δ 8.75 (s, 1H), |
| | | 8.50-8.44 (m, 2H), 8.17-8.09 (m, 3H), 6.97 (d, |
| | | J = 8.7 Hz, 2H), 5.08 (t, J = 5.6 Hz, 1H), 4.93- |
| | | 4.85 (m, 1H), 4.51-4.41 (m, 2H), 3.96 (s, 3H), |
| | | 3.85-3.75 (m, 1H), 3.72-3.64 (m, 1H), 3.52 (d, |
| | | J = 11.4 Hz, 2H), 2.90 (dd, J = 11.5, 2.6 Hz, 2H), |
| | | 1.92-1.81 (m, 4H), 1.44 (d, J = 6.9 Hz, 3H). |
| 5 | 446.4 | ¹H NMR (400 MHz, DMSO-d₆) δ 8.78 (s, 1H), |
| | | 8.52 (s, 1H), 8.49 (s, 1H), 8.24 (s, 1H), 8.20 (d, |
| | | J = 8.1 Hz, 2H), 7.42 (d, J = 8.2 Hz, 2H), 5.09 (t, |
| | | J = 5.6 Hz, 1H), 4.94-4.81 (m, 1H), 4.04-3.92 |
| | | (m, 5H), 3.90-3.75 (m, 1H), 3.75-3.64 (m, 1H), |
| | | 3.52-3.43 (m, 2H), 2.97-2.73 (m, 1H), 1.83- |
| | | 1.69 (m, 4H), 1.44 (d, J = 7.0 Hz, 3H). |
| 6 | 457.3 | ¹H NMR (400 MHz, DMSO-d₆) δ 9.57-9.51 (m, |
| | | 1H), 8.84 (s, 1H), 8.72 (dd, J = 8.4, 2.4 Hz, 1H), |
| | | 8.57 (s, 1H), 8.54 (s, 1H), 8.41 (s, 1H), 8.31-8.20 |
| | | (m, 2H), 8.11 (d, J = 8.4 Hz, 1H), 7.40-7.31 (m, |
| | | 2H), 5.10 (t, J = 5.6 Hz, 1H), 5.00-4.86 (m, 1H), |
| | | 3.98 (s, 3H), 3.90-3.79 (m, 1H), 3.75-3.61 (m, |
| | | 1H), 1.46 (d, J = 7.0 Hz, 3H). |
| 7 | 473.3 | ¹H NMR (400 MHz, DMSO-d₆) δ 9.56 (s, 1H), |
| | | 8.84 (s, 1H), 8.73 (dd, J = 8.4, 2.4 Hz, 1H), 8.56 |
| | | (d, J = 9.7 Hz, 2H), 8.42 (s, 1H), 8.23 (d, J = 8.6 |
| | | Hz, 2H), 8.14 (d, J = 8.3 Hz, 1H), 7.60 (d, J = 8.6 |
| | | Hz, 2H), 5.11 (t, J = 5.6 Hz, 1H), 4.99-4.84 (m, |
| | | 1H), 3.98 (s, 3H), 3.88-3.78 (m, 1H), 3.76-3.63 |
| | | (m, 1H), 1.46 (d, J = 6.9 Hz, 3H). |
| 8 | 458.3 | ¹H NMR (400 MHz, DMSO-d₆) δ 9.57 (d, J = 2.3 |
| | | Hz, 1H), 9.03 (d, J = 2.6 Hz, 1H), 8.84 (s, 1H), |
| | | 8.80-8.68 (m, 2H), 8.56 (d, J = 10.8 Hz, 2H), |
| | | 8.42 (s, 1H), 8.20 (d, J = 8.4 Hz, 1H), 7.36 (dd, |
| | | J = 8.6, 2.8 Hz, 1H), 5.11 (t, J = 5.6 Hz, 1H), |
| | | 4.94-4.86 (m, 1H), 3.97 (s, 3H), 3.88-3.75 (m, 1H), |
| | | 3.75-3.64 (m, 1H), 1.46 (d, J = 7.0 Hz, 3H). |
| 9 | 445.3 | ¹H NMR (400 MHz, DMSO-d₆) δ 9.44 (d, J = 2.4 |
| | | Hz, 1H), 8.83 (s, 1H), 8.62 (dd, J = 8.4, 2.4 Hz, |
| | | 1H), 8.56 (s, 1H), 8.53 (s, 1H), 8.36 (s, 1H), 7.70 |
| | | (d, J = 8.4 Hz, 1H), 6.93-6.89 (m, 1H), 5.10 (t, |
| | | J = 5.6 Hz, 1H), 4.96-4.85 (m, 1H), 4.35-4.29 |
| | | (m, 2H), 3.97 (s, 3H), 3.87 (t, J = 5.4 Hz, 2H), |
| | | 3.85-3.76 (m, 1H), 3.76-3.66 (m, 1H), 2.66- |
| | | 2.57 (m, 2H), 1.45 (d, J = 7.1 Hz, 3H). |
| 10 | 430.3 | ¹H NMR (400 MHz, DMSO-d₆) δ 8.83 (s, 1H), |
| | | 8.57 (s, 1H), 8.55 (s, 1H), 8.48-8.44 (m, 2H), |
| | | 8.06 (dd, J = 8.6, 1.4 Hz, 1H), 7.82 (d, J = 8.5 Hz, |
| | | 1H), 5.11 (t, J = 5.6 Hz, 1H), 4.98-4.87 (m, 1H), |
| | | 4.10 (s, 3H), 3.98 (s, 3H), 3.88-3.79 (m, 1H), |
| | | 3.75-3.66 (m, 1H), 2.52 (s, 3H), 1.46 (d, J = 7.0 |
| | | Hz, 3H). |
| 11 | 447.3 | ¹H NMR (400 MHz, DMSO-d₆) δ 9.39 (d, J = 2.5 |
| | | Hz, 1H), 8.83 (s, 1H), 8.61-8.55 (m, 2H), 8.53 |
| | | (s, 1H), 8.33 (s, 1H), 7.46 (d, J = 8.2 Hz, 1H), 5.11 |
| | | (t, J = 5.6 Hz, 1H), 4.95-4.86 (m, 1H), 4.03- |
| | | 3.96 (m, 5H), 3.87-3.78 (m, 1H), 3.73-3.66 (m, |
| | | 1H), 3.56-3.46 (m, 2H), 3.10-3.00 (m, 1H), |
| | | 1.89-1.79 (m, 4H), 1.46 (d, J = 7.0 Hz, 3H). |
| 12 | 457.3 | ¹H NMR (400 MHz, DMSO-d₆) δ 9.58 (d, J = 2.2 |
| | | Hz, 1H), 8.86 (s, 1H), 8.76 (dd, J = 8.5, 2.4 Hz, |
| | | 1H), 8.58 (s, 1H), 8.56 (s, 1H), 8.44 (s, 1H), 8.19 |
| | | (d, J = 8.6 Hz, 1H), 8.07 (d, J = 8.0 Hz, 1H), |
| | | 8.05-7.98 (m, 1H), 7.63-7.55 (m, 1H), 7.36-7.30 |
| | | (m, 1H), 5.11 (t, J = 5.6 Hz, 1H), 4.95-4.86 (m, |
| | | 1H), 3.98 (s, 3H), 3.88-3.79 (m, 1H), 3.74-3.65 |
| | | (m, 1H), 1.46 (d, J = 7.1 Hz, 3H). |
| 13 | 445.4 | ¹H NMR (400 MHz, DMSO-d₆) δ 8.77 (s, 1H), |
| | | 8.50-8.46 (m, 2H), 8.19-8.08 (m, 3H), 7.19- |
| | | 7.08 (m, 2H), 4.95-4.85 (m, 1H), 3.97 (s, 3H), |
| | | 3.85-3.78 (m, 1H), 3.71-3.67 (m, 1H), 3.65- |
| | | 3.40 (m, 1H), 3.37-3.27 (m, 4H), 1.73-1.56 (m, |
| | | 6H), 1.44 (d, J = 7.0 Hz, 3H). |
| 14 | 464.3 | ¹H NMR (400 MHz, DMSO-d₆) δ 8.65 (s, 1H), |
| | | 8.36 (s, 1H), 8.22 (s, 1H), 7.13-7.01 (m, 5H), |
| | | 5.06 (t, J = 5.6 Hz, 1H), 4.90-4.81 (m, 1H), 3.93 |
| | | (s, 3H), 3.84-3.74 (m, 5H), 3.70-3.62 (m, 1H), |
| | | 3.24 (t, J = 5.2 Hz, 4H), 1.40 (d, J = 7.0 Hz, 3H). |
| 15 | 403.3 | ¹H NMR (400 MHz, DMSO-d₆) δ 8.67 (s, 1H), |
| | | 8.40 (s, 1H), 8.19 (s, 1H), 7.48-7.42 (m, 2H), |
| | | 7.37-7.31 (m, 2H), 6.85 (s, 1H), 5.07 (t, J = 5.6 |
| | | Hz, 1H), 4.95-4.82 (m, 5H), 3.94 (s, 3H), 3.83- |
| | | 3.74 (m, 1H), 3.71-3.62 (m, 1H), 1.41 (d, J = 7.0 |
| | | Hz, 3H). |
| 16 | 461.3 | ¹H NMR (400 MHz, DMSO-d₆) δ 7.82 (s, 1H), |
| | | 7.53 (s, 1H), 7.36 (s, 1H), 6.76-6.65 (m, 2H), |
| | | 6.40-6.30 (m, 2H), 6.21 (s, 1H), 5.52-5.45 (m, |
| | | 1H), 4.22 (t, J = 5.5 Hz, 1H), 4.06-3.96 (m, 1H), |
| | | 3.44-3.34 (m, 2H), 3.19-3.11 (m, 2H), 3.10 (s, |
| | | 3H), 2.98-2.90 (m, 1H), 2.88-2.75 (m, 1H), |
| | | 1.85-1.75 (m, 2H), 0.55 (d, J = 7.1 Hz, 3H). |
| 17 | 490.3 | ¹H NMR (400 MHz, DMSO-d₆) δ 8.60 (s, 1H), |
| | | 8.31 (s, 1H), 8.18 (s, 1H), 7.08-7.00 (m, 2H), |
| | | 7.00-6.95 (m, 2H), 6.92 (s, 1H), 5.04 (t, J = 5.5 |
| | | Hz, 1H), 4.88-4.80 (m, 1H), 4.47-4.41 (m, 2H), |
| | | 3.97-3.90 (m, 5H), 3.79-3.70 (m, 1H), 3.68- |
| | | 3.60 (m, 1H), 3.23-3.16 (m, 2H), 2.00-1.95 (m, |
| | | 2H), 1.82 (d, J = 7.0 Hz, 2H), 1.38 (d, J = 7.0 Hz, |
| | | 3H). |
| 20 | 406.3 | 1H NMR (400 MHz, DMSO-d6) δ 8.79 (s, 1H), |
| | | 8.52-8.48 (m, 2H), 7.90-7.84 (m, 2H), 7.83- |
| | | 7.77 (m, 2H), 7.45 (d, J = 15.9 Hz, 1H), 7.26 (t, |
| | | J = 8.9 Hz, 2H), 5.10 (t, J = 5.6 Hz, 1H), 4.92-4.84 |
| | | (m, 1H), 3.96 (s, 3H), 3.84-3.75 (m, 1H), 3.72- |
| | | 3.63 (m, 1H), 1.43 (d, J = 7.1 Hz, 3H). |
| 21 | 475.37 | 1H NMR (400 MHz, DMSO-d6) δ ppm 8.81 (s, |
| | | 1H), 8.56 (s, 1H), 8.51 (s, 1H), 8.39-8.31 (m, |
| | | 3H), 7.57 (d, J = 8.0 Hz, 2H), 5.10 (t, J = 5.6 Hz, |
| | | 1H), 4.95-4.85 (m, 1H), 3.97 (s, 3H), 3.82 (dt, |
| | | J = 12.2, 6.3 Hz, 1H), 3.73-3.49 (m, 9H), 1.45 (d, |
| | | J = 7.1 Hz, 3H). |
| 25 | 448.32 | 1H NMR (400 MHz, DMSO-d6) δ ppm 9.05 (d, J = |
| | | 2.5 Hz, 1H), 8.79 (s, 1H), 8.49 (d, J = 1.7 Hz, |
| | | 2H), 8.42 (dd, J = 9.0, 2.5 Hz, 1H), 8.17 (s, 1H), |
| | | 6.96 (d, J = 8.9 Hz, 1H), 5.09 (t, J = 5.6 Hz, 1H), |
| | | 4.96-4.82 (m, 1H), 3.96 (s, 3H), 3.81 (dt, J = |
| | | 12.4, 6.3 Hz, 2H), 3.70 (dt, J = 22.0, 5.3 Hz, 6H), |
| | | 3.58 (s, 2H), 1.44 (d, J = 7.0 Hz, 3H). |
| 26 | 449.27 | 1H NMR (400 MHz, DMSO-d6) δ ppm 9.26 (s, |
| | | 2H), 8.82 (s, 1H), 8.50 (d, J = 4.2 Hz, 2H), 8.22 |
| | | (s, 1H), 5.10 (t, J = 5.6 Hz, 1H), 4.94-4.83 (m, |
| | | 1H), 3.95 (s, 3H), 3.81 (q, J = 6.3, 5.4 Hz, 5H), |
| | | 3.69 (dt, J = 10.1, 5.4 Hz, 5H), 1.44 (d, J = 7.1 Hz, |
| | | 3H). |
| 27 | 462.29 | 1H NMR (400 MHz, DMSO-d6) δ ppm 9.05 (d, J = |
| | | 2.5 Hz, 1H), 8.79 (s, 1H), 8.49 (d, J = 3.4 Hz, |
| | | 2H), 8.40 (dd, J = 9.0, 2.6 Hz, 1H), 8.16 (s, 1H), |
| | | 6.90 (d, J = 9.0 Hz, 1H), 5.10 (t, J = 5.6 Hz, 1H), |
| | | 4.89 (td, J = 7.1, 4.8 Hz, 1H), 4.50-4.38 (m, 1H), |
| | | 3.96 (s, 5H), 3.90-3.62 (m, 4H), 3.52 (td, J = |
| | | 11.7, 3.1 Hz, 1H), 3.15 (td, J = 12.8, 3.9 Hz, 1H), |
| | | 1.45 (d, J = 7.0 Hz, 3H), 1.18 (d, J = 6.6 Hz, 3H). |
| 28 | 488.35 | 1H NMR (400 MHz, DMSO-d6) δ ppm 8.80 (s, |
| | | 1H), 8.53 (d, J = 16.1 Hz, 2H), 8.38-8.26 (m, |
| | | 3H), 7.55 (t, J = 6.4 Hz, 2H), 5.12 (s, 1H), 4.89 |
| | | (p, J = 6.8 Hz, 1H), 3.97 (s, 3H), 3.82 (dd, J = |
| | | 11.7, 7.3 Hz, 2H), 3.75-3.61 (m, 4H), 3.02 (t, |
| | | J = 5.2 Hz, 1H), 2.86 (ddd, J = 26.4, 11.6, 5.5 Hz, |
| | | 3H), 1.77 (dt, J = 37.5, 6.1 Hz, 2H), 1.45 (d, J = |
| | | 7.1 Hz, 3H). |
| 29 | 475.32 | 1H NMR (400 MHz, DMSO-d6) δ ppm 8.81 (s, |
| | | 1H), 8.56 (s, 1H), 8.52 (s, 1H), 8.34 (d, J = 8.3 |
| | | Hz, 3H), 7.67 (dd, J = 8.3, 3.8 Hz, 2H), 5.10 (t, |
| | | J = 5.6 Hz, 1H), 5.06-5.03 (m, 0.4H), 4.97 (d, J = |
| | | 3.4 Hz, 0.6H), 4.91 (s, 1H), 4.35 (s, 0.5H), 4.26 |
| | | (s, 0.5H), 3.97 (s, 3H), 3.82 (dt, J = 12.4, 6.3 Hz, |
| | | 2H), 3.73-3.56 (m, 4H), 2.03-1.76 (m, 2H), |
| | | 1.45 (d, J = 7.1 Hz, 3H). |
| 30 | 488.30 | 1H NMR (400 MHz, DMSO-d6) δ ppm 8.80 (s, |
| | | 1H), 8.53 (d, J = 14.8 Hz, 2H), 8.37-8.28 (m, |
| | | 3H), 7.51 (d, J = 8.1 Hz, 2H), 5.11 (s, 1H), 4.89 |
| | | (dt, J = 13.9, 7.0 Hz, 1H), 4.31 (s, 1H), 3.97 (s, |
| | | 3H), 3.82 (t, J = 9.8 Hz, 1H), 3.69 (d, J = 11.4 Hz, |
| | | 2H), 3.08-2.91 (m, 2H), 2.88-2.77 (m, 1H), 1.74 |
| | | (d, J = 39.9 Hz, 2H), 1.45 (d, J = 7.0 Hz, 3H), 1.22 |
| | | (s, 2H). |
| 31 | 524.30 | 1H NMR (400 MHz, DMSO-d6) δ ppm 8.76 (s, |
| | | 1H), 8.47 (d, J = 2.9 Hz, 2H), 8.16 (t, J = 4.4 Hz, |
| | | 3H), 7.11 (d, J = 8.7 Hz, 2H), 4.89 (h, J = 7.0 Hz, |
| | | 1H), 3.96 (s, 3H), 3.81 (dd, J = 11.7, 7.1 Hz, 1H), |
| | | 3.68 (dd, J = 11.6, 4.6 Hz, 1H), 3.39 (d, J = 4.7 |
| | | Hz, 4H), 3.27 (t, J = 4.9 Hz, 4H), 2.94 (s, 3H), |
| | | 1.44 (d, J = 7.1 Hz, 3H). |
| 32 | 474.37 | 1H NMR (400 MHz, DMSO-d6) δ ppm 8.80 (s, |
| | | 1H), 8.53 (d, J = 20.6 Hz, 2H), 8.45-8.25 (m, |
| | | 3H), 7.60 (d, J = 7.9 Hz, 2H), 5.14 (s, 1H), 4.90 |
| | | (s, 1H), 3.97 (s, 3H), 3.88-3.65 (m, 6H), 3.05 (s, |
| | | 5H), 1.45 (d, J = 7.3 Hz, 3H). |
| 34 | 474.37 | 1H NMR (400 MHz, DMSO-d6) δ ppm 8.81 (s, |
| | | 1H), 8.56 (s, 1H), 8.51 (s, 1H), 8.40-8.31 (m, |
| | | 3H), 7.69 (t, J = 9.3 Hz, 4H), 5.11 (t, J = 5.6 Hz, |
| | | 1H), 4.97-4.81 (m, 1H), 3.97 (s, 3H), 3.91-3.65 |
| | | (m, 5H), 3.63-3.42 (m, 2H), 2.22 (s, 1H), 1.98 |
| | | (s, 1H), 1.45 (d, J = 7.1 Hz, 3H). |
| 35 | 489.30 | 1H NMR (400 MHz, DMSO-d6) δ ppm 8.80 (s, |
| | | 1H), 8.55 (s, 1H), 8.51 (s, 1H), 8.34 (d, J = 1.9 |
| | | Hz, 1H), 8.32 (d, J = 4.0 Hz, 2H), 7.55-7.49 (m, |
| | | 2H), 5.11 (t, J = 5.6 Hz, 1H), 4.90 (td, J = 7.1, 4.7 |
| | | Hz, 1H), 4.82 (d, J = 4.0 Hz, 1H), 3.97 (s, 4H), |
| | | 3.87-3.62 (m, 3H), 3.56 (s, 1H), 3.22 (s, 2H), |
| | | 1.78 (d, J = 32.6 Hz, 2H), 1.45 (d, J = 7.0 Hz, 5H). |
| 39 | 505.35 | 1H NMR (400 MHz, DMSO-d6) δ ppm 10.19 (s, |
| | | 1H), 9.22 (d, J = 2.4 Hz, 1H), 8.82 (s, 1H), 8.69 |
| | | (dd, J = 8.6, 2.5 Hz, 1H), 8.53 (d, J = 5.8 Hz, 2H), |
| | | 8.31 (s, 1H), 8.25 (d, J = 8.7 Hz, 1H), 5.10 (t, J = |
| | | 5.7 Hz, 1H), 4.90 (td, J = 7.2, 4.7 Hz, 1H), 3.97 |
| | | (s, 3H), 3.82 (dt, J = 12.3, 6.2 Hz, 1H), 3.68 (dt, |
| | | J = 20.2, 4.9 Hz, 5H), 3.25 (s, 2H), 2.57 (t, J = 4.6 |
| | | Hz, 4H), 1.45 (d, J = 7.1 Hz, 3H). |
| 41 | 458.31 | 1H NMR (400 MHz, DMSO-d6) δ ppm 9.63 (d, J = |
| | | 2.3 Hz, 1H), 9.29 (d, J = 2.1 Hz, 1H), 8.86 (s, |
| | | 1H), 8.81 (dd, J = 8.3, 2.4 Hz, 1H), 8.70 (d, J = |
| | | 2.8 Hz, 1H), 8.57 (d, J = 8.6 Hz, 2H), 8.45 (d, J = |
| | | 10.0 Hz, 2H), 8.30 (d, J = 8.2 Hz, 1H), 5.11 (t, |
| | | J = 5.6 Hz, 1H), 4.92 (d, J = 12.2 Hz, 1H), 3.98 (s, |
| | | 3H), 3.83 (dt, J = 12.2, 6.0 Hz, 1H), 3.70 (dt, J = |
| | | 11.1, 5.2 Hz, 1H), 1.46 (d, J = 7.1 Hz, 3H). |
| 42 | 504.36 | 1H NMR (400 MHz, CD3OD) δ ppm 8.75 (s, |
| | | 1H), 8.53 (s, 1H), 8.42 (s, 1H), 8.27 (s, 1H), |
| | | 8.21-8.16 (m, 2H), 7.79-7.70 (m, 2H), 4.99 (td, J = |
| | | 7.1, 4.4 Hz, 1H), 4.03-3.93 (m, 4H), 3.86 (dd, J = |
| | | 11.8, 4.4 Hz, 1H), 3.82-3.75 (m, 4H), 3.23 (s, |
| | | 2H), 2.64 (t, J = 4.7 Hz, 4H), 1.56 (d, J = 7.0 Hz, |
| | | 3H). |
| 44 | 469.26 | 1H NMR (400 MHz, DMSO-d6) δ ppm 8.80 (s, |
| | | 1H), 8.52 (d, J = 11.0 Hz, 2H), 8.30 (dd, J = 14.6, |
| | | 11.3 Hz, 4H), 7.93 (d, J = 9.4 Hz, 1H), 7.75 (d, |
| | | J = 8.1 Hz, 2H), 6.53 (d, J = 9.4 Hz, 1H), 5.12 (s, |
| | | 1H), 4.90 (s, 1H), 3.97 (s, 3H), 3.81 (s, 1H), 3.70 |
| | | (s, 1H), 3.55 (s, 3H), 1.45 (d, J = 7.0 Hz, 3H). |
| 52 | 421.3 | 1H NMR (400 MHz, DMSO-d6) δ ppm 8.67 (s, |
| | | 1H), 8.41 (s, 1H), 8.20 (s, 1H), 7.41 (q, J = 7.1, |
| | | 6.7 Hz, 1H), 7.31 (d, J = 7.5 Hz, 1H), 7.17 (t, J = |
| | | 8.8 Hz, 1H), 6.88 (s, 1H), 5.07 (t, J = 5.7 Hz, 1H), |
| | | 5.01-4.78 (m, 5H), 3.94 (s, 3H), 3.79 (s, 1H), |
| | | 3.72-3.58 (m, 1H), 1.41 (d, J = 7.0 Hz, 3H). |
| 54 | 461.35 | 1H NMR (400 MHz, DMSO-d6) δ ppm 8.69 (s, |
| | | 1H), 8.51 (s, 1H), 8.36 (s, 1H), 7.80 (s, 1H), 7.46 |
| | | (d, J = 9.0 Hz, 1H), 6.91 (d, J = 7.2 Hz, 2H), 5.07 |
| | | (t, J = 5.6 Hz, 1H), 4.88 (s, 1H), 3.94 (s, 3H), |
| | | 3.85-3.73 (m, 5H), 3.72-3.61 (m, 1H), 3.19 (t, J = |
| | | 4.9 Hz, 4H), 2.45 (s, 3H), 1.44 (d, J = 7.1 Hz, 3H). |
| 56 | 421.30 | 1H NMR (400 MHz, DMSO-d6) δ ppm 8.65 (s, |
| | | 1H), 8.38 (s, 1H), 8.19 (s, 1H), 7.54-7.41 (m, |
| | | 1H), 7.29 (d, J = 9.2 Hz, 1H), 7.16 (t, J = 9.0 Hz, |
| | | 1H), 6.82 (s, 1H), 5.07 (t, J = 5.6 Hz, 1H), 4.85 |
| | | (d, J = 15.9 Hz, 5H), 3.94 (s, 3H), 3.77 (d, J = 12.1 |
| | | Hz, 1H), 3.66 (d, J = 12.5 Hz, 1H), 1.41 (d, J = |
| | | 7.0 Hz, 3H). |
| 57 | 461.35 | 1H NMR (400 MHz, DMSO-d6) δ ppm 8.77 (s, |
| | | 1H), 8.50 (d, J = 3.5 Hz, 2H), 8.20 (s, 1H), 8.10- |
| | | 8.02 (m, 2H), 7.15 (d, J = 8.4 Hz, 1H), 5.09 (t, J = |
| | | 5.6 Hz, 1H), 4.89 (td, J = 7.2, 4.7 Hz, 1H), 3.97 |
| | | (s, 3H), 3.86-3.73 (m, 5H), 3.68 (dt, J = 11.2, |
| | | 5.1 Hz, 1H), 2.92 (t, J = 4.5 Hz, 4H), 2.39 (s, 3H), |
| | | 1.44 (d, J = 7.1 Hz, 3H). |
| 119 | 456.33 | 1H NMR (400 MHz, DMSO-d6) δ ppm 9.48 (s, |
| | | 1H), 9.05 (s, 2H), 8.83 (s, 1H), 8.67 (d, J = 8.3 |
| | | Hz, 1H), 8.54 (d, J = 9.0 Hz, 2H), 8.38 (s, 1H), |
| | | 8.04 (d, J = 8.5 Hz, 1H), 7.54 (s, 1H), 7.17 (s, 1H), |
| | | 4.90 (s, 1H), 3.97 (s, 3H), 3.82 (s, 2H), 1.45 (d, |
| | | J = 6.9 Hz, 3H). |
| 120 | 552.25 | 1H NMR (400 MHz, DMSO-d6) δ ppm 8.80 (s, |
| | | 1H), 8.55 (s, 1H), 8.51 (s, 1H), 8.39-8.30 (m, |
| | | 3H), 7.59 (d, J = 7.9 Hz, 2H), 5.11 (t, J = 5.6 Hz, |
| | | 1H), 4.89 (d, J = 11.1 Hz, 1H), 3.97 (s, 3H), 3.87- |
| | | 3.46 (m, 6H), 3.20 (s, 4H), 2.92 (s, 3H), 1.45 (d, |
| | | J = 7.0 Hz, 3H). |
| 121 | 448.32 | 1H NMR (400 MHz, DMSO-d6) δ ppm 8.66 (s, |
| | | 1H), 8.41 (d, J = 4.7 Hz, 2H), 8.37 (s, 1H), 8.21 |
| | | (s, 1H), 7.09 (s, 1H), 6.67 (t, J = 4.7 Hz, 1H), 5.05 |
| | | (t, J = 5.6 Hz, 1H), 4.91-4.73 (m, 1H), 3.92 (d, |
| | | J = 9.4 Hz, 7H), 3.79-3.63 (m, 6H), 1.40 (d, J = |
| | | 7.1 Hz, 3H). |
| 122 | 482.28 | 1H NMR (400 MHz, DMSO-d6) δ ppm 8.65 (s, |
| | | 1H), 8.36 (s, 1H), 8.22 (s, 1H), 7.30-6.96 (m, |
| | | 4H), 5.05 (t, J = 5.5 Hz, 1H), 4.85 (s, 1H), 3.93 (s, |
| | | 3H), 3.85-3.72 (m, 5H), 3.70-3.59 (m, 1H), |
| | | 3.18-3.04 (m, 4H), 1.40 (d, J = 7.0 Hz, 3H). |
| 123 | 446.38 | 1H NMR (400 MHz, DMSO-d6) δ ppm 8.66 (s, |
| | | 1H), 8.36 (s, 1H), 8.22 (s, 1H), 7.29-7.22 (m, |
| | | 2H), 7.10 (s, 1H), 7.05-6.99 (m, 2H), 6.82 (t, |
| | | J = 7.3 Hz, 1H), 5.06 (t, J = 5.5 Hz, 1H), 4.85 (q, |
| | | J = 7.0 Hz, 1H), 3.93 (s, 3H), 3.77 (q, J = 6.6, 5.8 |
| | | Hz, 5H), 3.65 (dt, J = 10.9, 5.1 Hz, 1H), 3.30 (d, |
| | | J = 7.3 Hz, 4H), 1.40 (d, J = 7.1 Hz, 3H). |
| 124 | 404.31 | 1H NMR (400 MHz, DMSO-d6) δ ppm 8.69 (s, |
| | | 1H), 8.55-8.48 (m, 1H), 8.41 (s, 1H), 8.20 (s, |
| | | 1H), 7.88 (d, J = 7.9 Hz, 1H), 7.41-7.32 (m, 1H), |
| | | 6.90 (s, 1H), 5.07 (t, J = 5.5 Hz, 1H), 4.94 (s, 2H), |
| | | 4.87 (s, 3H), 3.95 (s, 3H), 3.79 (s, 1H), 3.69-3.65 |
| | | (m, 1H), 1.41 (d, J = 7.0 Hz, 3H). |
| 128 | 410.3 | ¹H NMR (400 MHz, CD₃OD) δ 9.20 (d, J = 1.7 |
| | | Hz, 1H),9.03 (s, 1H), 8.73 (d, J = 10.0 Hz, |
| | | 1H), 8.56 (d, J = 2.7 Hz, 1H), 8.53 (s, 1H), |
| | | 8.43-8.36 (m, 1H), 7.34 (d, J = 10.0 Hz, |
| | | 1H), 4.37-4.28 (m, 1H), 3.78-3.68 (m, 2H), |
| | | 1.38 (d, J = 6.8 Hz, 3H). |
| 129 | 407.3 | ¹H NMR (400 MHz, DMSO-d6) δ 9.34 (t, J = 1.7 Hz, |
| | | 1H), 8.90 (d, J = 1.3 Hz, 1H), 8.69 (d, J = 2.7 |
| | | Hz, 1H), 8.65 (s, 1H), 8.63-8.58 (m, 2H), 8.12 |
| | | (d, J = 1.3 Hz, 1H), 5.11 (t, J = 5.6 Hz, 1H), |
| | | 4.91 (td, J = 7.3, 4.8 Hz, 1H), 3.82 (dt, J = |
| | | 12.3, 6.5 Hz, 1H), 3.69 (dt, J = 11.1, 5.2 Hz, |
| | | 1H), 1.45 (d, J = 7.1 Hz, 3H), 0.85 (ddt, |
| | | J = 8.3, 5.4, 3.0 Hz, 2H), 0.80-0.75 (m, 2H). |

### Preparation of 1-methyl-1H-pyrazole-4-carboximidamide (AA-2)

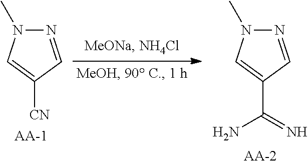

To a solution of 1-methyl-1H-pyrazole-4-carbonitrile, AA-1 (4.0 g, 37.37 mmol) in methanol (40 mL) was added a solution of 28% sodium methoxide in methanol (6.6 ml). After stirred at 25° C. for 3 h. Then, ammonium chloride (12.0 g, 226.42 mmol) was added. The mixture was stirred at 90° C. for 1 h. The desired mass was detected on LC-MS. The reaction solution was concentrated under reduced pressure to get crude product which was purified by flash chromatography (eluting with DCM/MeOH from 100/0 to 70/30 in 20 mins) to afford AA-2 (4.0 g, yield: 86.3%) as a white solid. LCMS (ESI): calced for C₅H₉N₄ [M+H]⁺ m/z 125.1, found 125.2.

### Preparation of 6-hydroxy-2-(1-methyl-1H-pyrazol-4-yl)pyrimidine-4-carboxylic acid (AA-4)

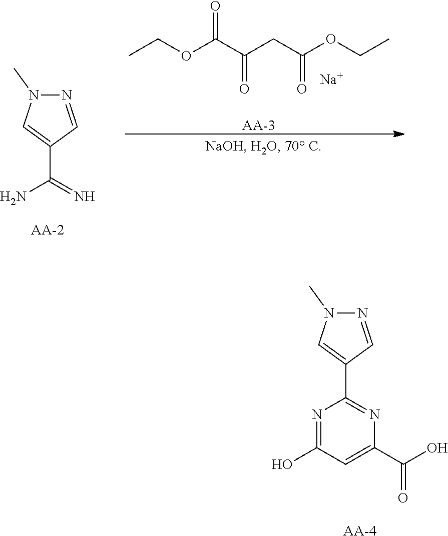

To a solution of AA-3 (8.2 g, 38.71 mmol) in water (80 mL) was added sodium methoxide (3.1 g, 77.42 mmol) in water (3 mL). After 10 minutes, AA-2 (4.0 g, 32.24 mmol) was added. The reaction mixture was stirred at 70° C. for 16 h. The desired mass was detected on LC-MS. After cooled down to room temperature, the reaction solution was adjusted to pH=4 with 6 M HCl. Filtrated out and collected solids to get product AA-4 (3.0 g, yield: 42.3%) as a brown solid. LCMS (ESI): calced for C₉H₉N₄O₃ [M+H]⁺ m/z 221.1, found 221.1.

### Preparation of methyl 6-hydroxy-2-(1-methyl-1H-pyrazol-4-yl)pyrimidine-4-carboxylate (AA-5)

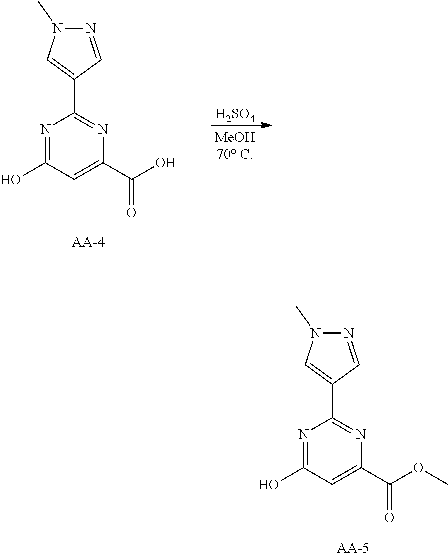 [SMILES: Cn1cc(-c2nc(O)cc(C(=O)O)n2)cn1 ; "H2SO4" ; "AA-4" ; COC(=O)c1cc(O)nc(-c2cnn(C)c2)n1 ; "AA-5"]

To a solution of AA-4 (3.0 g, 13.63 mmol) in MeOH (30 mL) was added sulfuric acid (2 mL). The mixture was stirred at 70° C. for 2 h. The desired mass was detected on LC-MS. The resulting solution was filtered out and collected solids to get product AA-5 (2.8 g, yield: 97.7%) as a white solid. LCMS (ESI): calced for C₁₀H₁₁N₄O₃ [M+H]⁺ m/z 235.1, found 235.2.

### Preparation of methyl 6-chloro-2-(1-methyl-1H-pyrazol-4-yl)pyrimidine-4-carboxylate (AA)

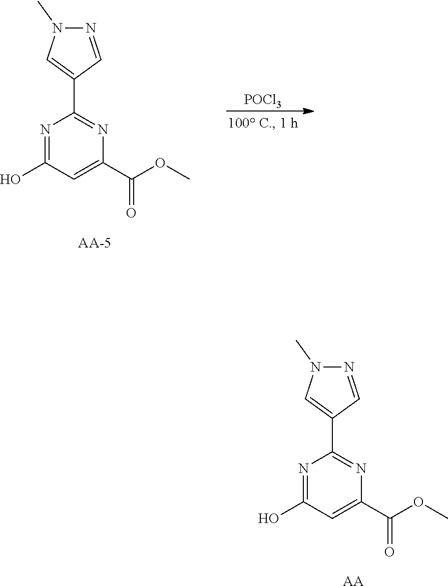 [SMILES: COC(=O)c1cc(O)nc(-c2cnn(C)c2)n1 ; "POCl3" ; "AA-5" ; COC(=O)c1cc(O)nc(-c2cnn(C)c2)n1 ; "AA"]

To a solution of AA-5 (2.8 g, 11.96 mmol) was added phosphoryl trichloride (60 mL). The mixture was stirred at 100° C. for 1 h. The desired mass was detected on LC-MS. The reaction solution was concentrated under reduced pressure and the solution was adjusted to pH=7 with saturated sodium bicarbonate solution, extracted with EtOAc. the organic layer was concentrated to crude product AA (2.2 g, yield: 93.9%) as a white solid. LCMS (ESI): calced for C₁₀H₁₀N₄O₃ [M+H]⁺ m/z 253.0, found 253.2.

### Preparation of Compound (C-3)

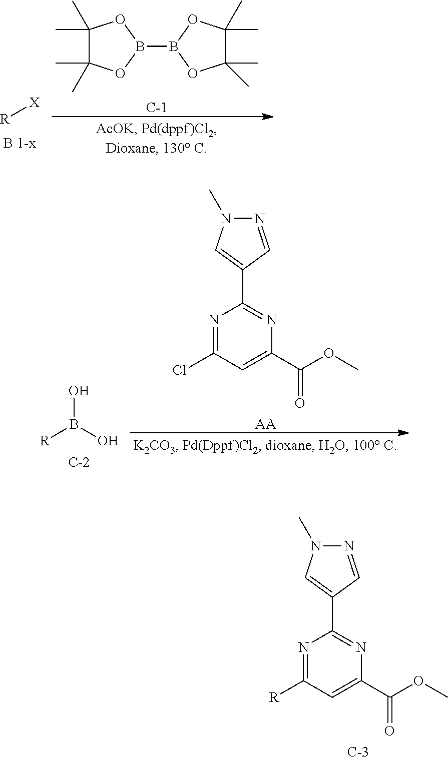

To a solution of compound (B 1-x) (1.0 eq), compound (C-1) (1.2 eq), Potassium acetate (1.5 eq) and Pd(dppf)Cl₂ (0.01 eq) in 1,4-dioxane (10 ml). The reaction mixture was stirred at 80° C. for 16 h under N₂. The desired mass was detected on LC-MS and was added compound (AA) (0.8 eq), Potassium carbonate (2.0 eq) and Pd(dppf)Cl₂ (0.01 eq) and H₂O (3 ml). The reaction mixture was stirred at 80° C. for 16 h under N₂. The desired mass was detected on LC-MS. The solvent was removed by vacuum. The residue was purified by flash chromatography (eluting with DCM/MeOH from 100/0 to 90/10 in 30 mins) to afford compound (C-3).

### Preparation of Compound (C-4)

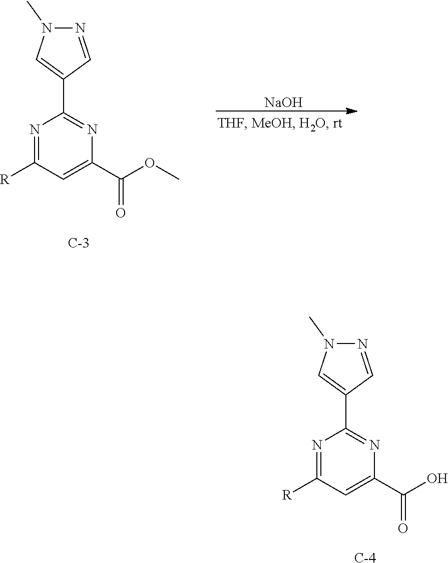

To a solution of compound (C-3) (1.0 eq) in (MeOH:THF:H₂O=6:3:1, 10 mL) was added sodium hydroxide (2.0 eq). The mixture was stirred at RT for 4 h. The desired mass was detected on LC-MS. The reaction solution was concentrated under reduced pressure and adjusted to pH=4 with 2 N HCl. The solution was filtered and solids collected to get compound (C-4).

### Preparation of Compound of the Disclosure

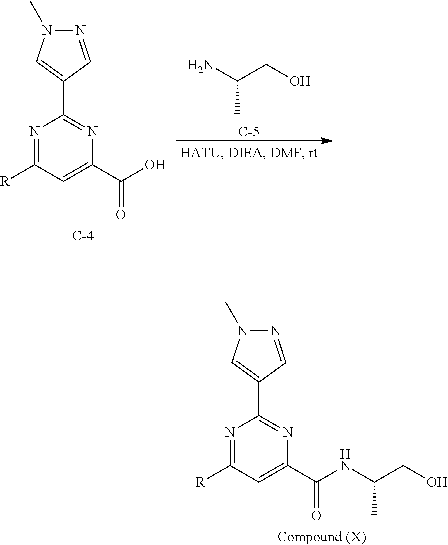

To a solution of compound (C-4) (1.0 eq) in DMF (5 ml) was added HATU (2.0 eq) and DIEA (3.0 eq). After 30 min, compound (C-5) was added. The reaction mixture was stirred at RT for 2 h. The desired mass was detected on LC-MS. The resulting was then quenched to water, extracted with EtOAc, washed with saturated brine, dried over Na₂SO₄ and concentrated. The residue was purified by flash chromatography (eluting with DCM/MeOH from 100/0 to 90/10 in 30 mins) to afford the desired compound.

### Preparation of Compound (D-2)

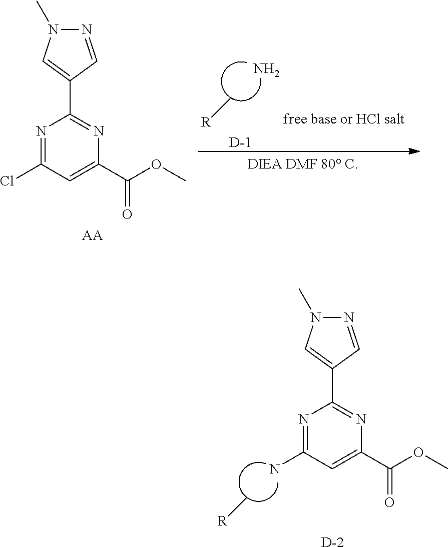

To a solution of compound AA (1.0 eq) in DMF (5 ml) was added compound (D-1) (1.2 eq) and DIEA (3.0 eq). The reaction mixture was stirred at 80° C. for 2 h. The desired mass was detected on LC-MS. The resulting was then quenched to water, extracted with EtOAc, washed with saturated brine, dried over Na₂SO₄ and concentrated. The residue was purified by flash chromatography (eluting with DCM/MeOH from 100/0 to 90/10 in 30 mins) to afford compound (D-2).

### Preparation of Compound (D-3)

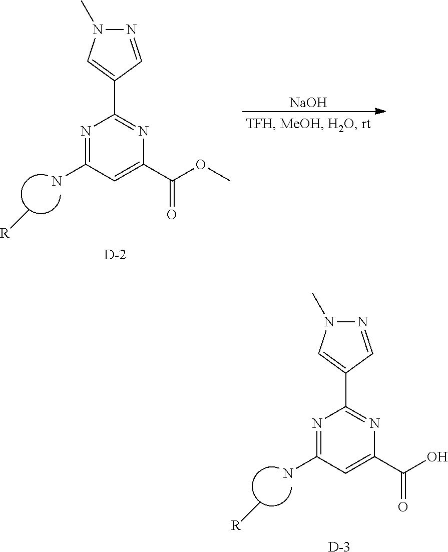

To a solution of compound (D-2) (1.0 eq) in (MeOH:THF:H₂O=6:3:1, 10 mL) was added sodium hydroxide (2.0 eq). The mixture was stirred at RT for 4 h. The desired mass was detected on LC-MS. The reaction solution was concentrated under reduced pressure and adjusted to pH=4 with 2 N HCl. The solution was filtered, and the solids collected to get compound (D-3).

### Preparation of a Compound of the Disclosure

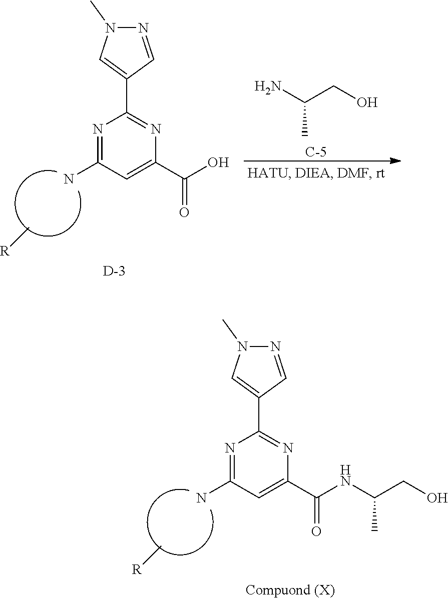

To a solution of compound (D-3) (1.0 eq) in DMF (5 ml) was added HATU (2.0 eq) and DIEA (3.0 eq). After 30 min, compound (C-5) was added. The reaction mixture was stirred at RT for 2 h. The desired mass was detected on LC-MS. The resulting was then quenched to water, extracted with EtOAc, washed with saturated brine, dried over Na₂SO₄ and concentrated. The residue was purified by flash chromatography (eluting with DCM/MeOH from 100/0 to 90/10 in 30 mins) to afford the desired compound.

The compounds in the following table were prepared in a manner similar to that as described above.

| | LCMS | |
|---|---|---|
| | (ESI): | |
| | [M + H]⁺ | Analytics |
| 59 | 433.30 | ¹H NMR (400 MHZ, DMSO-d₆) δ 9.65 (d, J = 2.3 Hz, |
| | | 1H), 8.82 (dd, J = 8.4, 2.4 Hz, 1H), 8.71 (s, 1H), 8.62 |
| | | (d, J = 8.5 Hz, 1H), 8.40 (s, 1H), 8.35 (s, 1H), 8.11- |
| | | 7.98 (m, 2H), 7.56 (tdd, J = 7.3, 5.0, 1.8 Hz, 1H), |
| | | 7.44-7.35 (m, 2H), 4.91 (t, J = 5.7 Hz, 1H), 4.12 (dt, |
| | | J = 14.7, 6.4 Hz, 1H), 3.97 (s, 3H), 3.62-3.46 (m, 2H), |
| | | 1.25 (s, 3H). |
| 60 | 440.3 | ¹H NMR (400 MHZ, DMSO-d₆) δ 8.42 (s, 1H), 8.39 |
| | | (d, J = 8.5 Hz, 1H), 8.15 (s, 1H), 7.15 (s, 1H), 7.12- |
| | | 7.07 (m, 2H), 7.05-7.00 (m, 2H), 4.90 (t, J = 5.6 Hz, |
| | | 1H), 4.08-3.98 (m, 1H), 3.92 (s, 3H), 3.91-3.84 |
| | | (m, 4H), 3.57-3.42 (m, 2H), 3.24-3.16 (m, 4H), |
| | | 1.19 (d, J = 6.7 Hz, 3H). |
| 61 | 433.30 | ¹H NMR (400 MHZ, DMSO-d₆) δ 9.59 (d, J = 2.3 Hz, |
| | | 1H), 8.81-8.75 (m, 1H), 8.70 (s, 1H), 8.61 (d, J = |
| | | 8.7 Hz, 1H), 8.39 (s, 1H), 8.34 (s, 1H), 8.31-8.25 |
| | | (m, 2H), 8.17 (d, J = 8.4 Hz, 1H), 7.41-7.34 (m, |
| | | 2H), 4.96-4.86 (m, 1H), 3.97 (s, 3H), 3.64-3.44 |
| | | (m, 2H), 1.25 (s, 3H). |
| 62 | 423.30 | ¹H NMR (400 MHZ, DMSO-d₆) δ 8.60 (s, 1H), 8.53 |
| | | (d, J = 8.6 Hz, 1H), 8.31 (s, 1H), 8.23 (d, J = 8.8 Hz, |
| | | 2H), 8.08 (s, 1H), 7.08 (d, J = 8.9 Hz, 2H), 4.90 (t, |
| | | J = 5.6 Hz, 1H), 4.14-4.04 (m, 1H), 3.95 (s, 3H), |
| | | 3.81-3.72 (m, 4H), 3.59-3.44 (m, 2H), 3.31-3.27 (m, |
| | | 4H), 1.26-1.22 (m, 3H). |
| 63 | 379.30 | ¹H NMR (400 MHZ, DMSO-d₆) δ 8.42 (d, J = 7.1 Hz, |
| | | 2H), 8.17 (s, 1H), 7.49-7.40 (m, 2H), 7.37-7.32 |
| | | (m, 2H), 6.95 (s, 1H), 5.00-4.83 (m, 5H), 4.09-3.99 |
| | | (m, 1H), 3.93 (s, 3H), 3.57-3.43 (m, 2H), 1.28-1.18 |
| | | (m,3H). |
| 64 | 393.30 | ¹H NMR (400 MHZ, DMSO-d₆) δ 8.78 (s, 1H), 8.57 |
| | | (s, 1H), 8.52 (d, J = 8.5 Hz, 1H), 8.28 (s, 1H), 7.88 |
| | | (d, J = 7.6 Hz, 1H), 7.79-7.72 (m, 2H), 7.62-7.55 |
| | | (m, 1H), 5.21 (s, 2H), 4.91 (t, J = 5.6 Hz, 1H), 4.11- |
| | | 4.03 (m, 1H), 3.95 (s, 3H), 3.57-3.46 (m, 2H), 1.22 |
| | | (d, J = 6.7 Hz, 3H). |
| 65 | 493.467 | ¹H NMR (400 MHz, DMSO) δ 9.23 (s, 1H), 8.68 (s, |
| | | 1H), 8.46 (d, J = 8.7 Hz, 1H), 7.26 (s, 1H), 7.13- |
| | | 7.06 (m, 2H), 7.06-6.98 (m, 2H), 4.87 (t, J = 5.6 Hz, |
| | | 1H), 4.09-4.01 (m, 1H), 3.93 (s, 3H), 3.55-3.41 |
| | | (m, 2H), 3.20 (t, J = 5.1 Hz, 4H), 1.20 (d, J = 6.8 Hz, |
| | | 3H). |
| 66 | 453.494 | ¹H NMR (400 MHz, DMSO) δ 8.54 (d, J = 8.6 Hz, |
| | | 1H), 8.34 (d, J = 1.3 Hz, 1H), 8.32-8.27 (m, 1H), |
| | | 7.60-7.53 (m, 1H), 7.40-7.36 (m, 1H), 7.36-7.32 |
| | | (m, 1H), 7.12-7.06 (m, 2H), 7.05-6.98 (m, 2H), |
| | | 4.89 (t, J = 5.6 Hz, 1H), 4.10-4.02 (m, 1H), 3.95 (s, |
| | | 3H), 3.56-3.43 (m, 2H), 3.22 (t, J = 5.2 Hz, 4H), |
| | | 1.20 (d, J = 6.7 Hz, 3H). |
| 67 | 406.35 | 1H NMR (400 MHz, DMSO-d6) δ 8.69 (s, 1H), 8.63- |
| | | 8.59 (m, 2H), 8.41 (d, J = 6.0 Hz, 2H), 8.14-8.08 |
| | | (m, 1H), 7.87 (d, J = 8.5 Hz, 1H), 5.01-4.87 (m, |
| | | 1H), 4.12 (s, 3H), 3.98 (s, 3H), 3.61-3.46 (m, 2H), |
| | | 2.53 (s, 3H), 1.24-1.22 (m, 3H). |
| 68 | 433.30 | ¹H NMR (400 MHz, DMSO-d₆) δ 9.62 (d, J = 2.3 Hz, |
| | | 1H), 8.82 (dd, J = 8.4, 2.3 Hz, 1H), 8.71 (s, 1H), 8.63 |
| | | (d, J = 8.6 Hz, 1H), 8.40 (s, 1H), 8.36 (s, 1H), 8.24 |
| | | (d, J = 8.5 Hz, 1H), 8.13-8.00 (m, 2H), 7.64-7.56 |
| | | (m, 1H), 7.40-7.31 (m, 1H), 4.14-4.07 (m, 1H), |
| | | 3.97 (s, 3H), 3.59-3.52 (m, 3H), 1.23 (s, 3H). |
| 69 | 434.25 | ¹H NMR (400 MHz, DMSO-d₆) δ 9.64 (d, J = 2.3 Hz, |
| | | 1H), 9.07 (d, J = 2.5 Hz, 1H), 8.88-8.83 (m, 1H), |
| | | 8.81-8.73 (m, 1H), 8.72 (s, 1H), 8.63 (d, J = 8.6 Hz, |
| | | 1H), 8.39 (d, J = 14.1 Hz, 2H), 8.28 (d, J = 8.4 Hz, |
| | | 1H), 7.42-7.35 (m, 1H), 3.97 (s, 3H), 3.59-3.47 |
| | | (m, 3H), 1.23 (s, 3H). |
| 70 | 449.28 | 1H NMR (400 MHz, DMSO-d6) δ 9.52 (d, J = 2.2 |
| | | Hz, 1H), 8.56 (dd, J = 8.4, 2.3 Hz, 1H), 8.33 (s, 1H), |
| | | 8.27 (s, 1H), 8.19 (d, J = 12.5 Hz, 2H), 8.08-8.03 |
| | | (m, 2H), 7.87 (d, J = 8.4 Hz, 1H), 7.52-7.46 (m, |
| | | 2H), 4.39-4.27 (m, 1H), 4.02 (s, 3H), 3.92-3.71 |
| | | (m, 2H), 1.40 (d, J = 6.8 Hz, 3H). |
| 71 | 422.37 | 1H NMR (400 MHz, DMSO-d6) δ 8.63 (s, 1H), 8.59 |
| | | (d, J = 8.6 Hz, 1H), 8.34 (s, 1H), 8.31-8.25 (m, 2H), |
| | | 8.18 (s, 1H), 7.50-7.44 (m, 2H), 4.91 (s, 1H), 4.15- |
| | | 4.05 (m, 1H), 4.00-3.97 (m, 1H), 3.96 (s, 3H), 3.61- |
| | | 3.42 (m, 4H), 2.95-2.82 (m, 1H), 1.78-1.68 (m, |
| | | 4H), 1.22 (d, J = 6.7 Hz, 3H). |
| 72 | 454.35 | 1H NMR (400 MHz, DMSO-d6) δ 8.42 (d, J = 10.4 |
| | | Hz, 2H), 8.16 (s, 1H), 7.17 (s, 1H), 7.14-6.95 (m, |
| | | 4H), 4.07-3.97 (m, 2H), 3.91 (s, 3H), 3.76-3.58 |
| | | (m, 5H), 3.48 (s, 4H), 1.18 (d, J = 6.7 Hz, 3H), 0.91 |
| | | (d, J = 6.4 Hz, 3H). |
| 73 | 454.38 | 1H NMR (400 MHz, Methanol-d4) δ 8.37 (s, 1H), |
| | | 8.18 (s, 1H), 7.24 (s, 1H), 7.20-7.11 (m, 2H), |
| | | 7.10-7.02 (m, 2H), 4.24-4.17 (m, 1H), 4.17-3.98 (m, |
| | | 2H), 3.96 (s, 3H), 3.93-3.75 (m, 3H), 3.66 (d, J = |
| | | 5.2 Hz, 2H), 3.29-3.19 (m, 2H), 1.29 (d, J = 6.8 Hz, |
| | | 3H), 1.00 (d, J = 6.3 Hz, 3H). |
| 74 | 466.36 | 1H NMR (400 MHz, DMSO-d6) δ 8.37 (d, J = 6.2 |
| | | Hz, 2H), 8.10 (s, 1H), 7.09-7.00 (m, 3H), 6.99- |
| | | 6.92 (m, 2H), 4.40 (d, J = 3.8 Hz, 2H), 4.06-3.96 |
| | | (m, 1H), 3.90 (s, 3H), 3.52-3.41 (m, 5H), 1.94 (dd, |
| | | J = 9.1, 4.4 Hz, 2H), 1.71 (d, J = 7.9 Hz, 2H), 1.17 (d, |
| | | J = 6.7 Hz, 3H). |
| 75 | 468.34 | 1H NMR (400 MHz, DMSO-d6) δ 8.43 (s, 1H), 8.40 |
| | | (d, J = 8.6 Hz, 1H), 8.13 (s, 1H), 7.58-7.50 (m, 2H), |
| | | 7.31 (t, J = 8.9 Hz, 2H), 7.10 (s, 1H), 4.95 (t, J = 5.6 |
| | | Hz, 1H), 4.06-3.96 (m, 1H), 3.90 (s, 3H), 3.87- |
| | | 3.57 (m, 6H), 3.53-3.42 (m, 4H), 1.17 (d, J = 6.7 |
| | | Hz, 3H). |
| 76 | 454.37 | 1H NMR (400 MHz, DMSO-d6) δ 8.41 (s, 1H), 8.39 |
| | | (d, J = 8.6 Hz, 1H), 8.14 (d, J = 0.8 Hz, 1H), 7.14 (s, |
| | | 1H), 7.11-7.05 (m, 2H), 7.01-6.95 (m, 2H), 4.91 |
| | | (t, J = 5.6 Hz, 1H), 4.59-4.13 (m, 1H), 4.08-3.92 |
| | | (m, 2H), 3.91 (s, 3H), 3.67-3.39 (m, 4H), 3.32-3.21 |
| | | (m, 2H), 3.12-2.97 (m, 1H), 1.18 (d, J = 6.7 Hz, |
| | | 3H), 0.89 (d, J = 6.4 Hz, 3H). |
| 77 | 454.37 | 1H NMR (400 MHz, DMSO-d6) δ 8.42 (d, J = 6.6 |
| | | Hz, 2H), 8.15 (s, 1H), 7.13-7.05 (m, 3H), 7.04- |
| | | 6.97 (m, 2H), 4.48 (s, 1H), 4.09-3.98 (m, 1H), 3.91 |
| | | (s, 3H), 3.54-3.32 (m, 7H), 2.99-2.90 (m, 1H), 2.81- |
| | | 2.70 (m, 1H), 1.30 (d, J = 6.6 Hz, 3H), 1.18 (d, J = |
| | | 6.7 Hz, 3H). |

### Preparation of Compound (E-2)

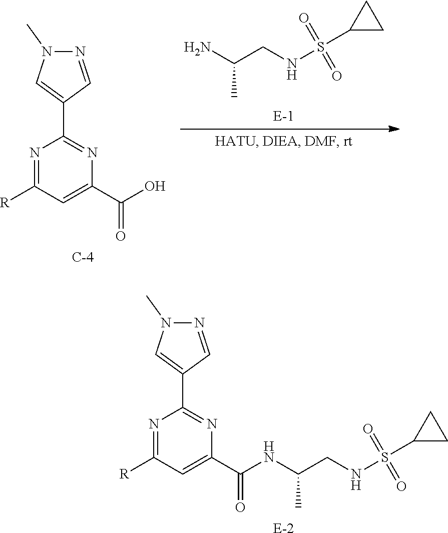

To a solution of compound (C-4) (1.0 eq) in DMF (5 ml) was added HATU (2.0 eq) and DIEA (3.0 eq). After 30 min, compound (E-1) was added. The reaction mixture was stirred at RT for 2 h. The desired mass was detected on LC-MS. The resulting was then quenched to water, extracted with EtOAc, washed with saturated brine, dried over Na₂SO₄ and concentrated. The residue was purified by flash chromatography (eluting with DCM/MeOH from 100/0 to 90/10 in 30 mins) to afford the compound (E-2)

### Preparation of Compound (X)

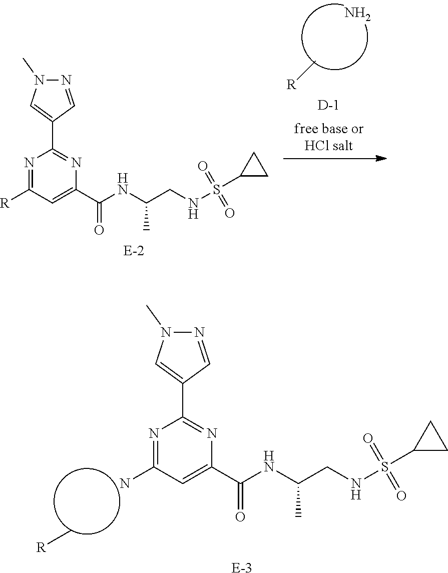

To a solution of compound (E-2) (1.0 eq) in DMF (5 ml) was added compound (E-5) (1.2 eq) and K₂CO₃ (3.0 eq). The reaction mixture was stirred at 80° C. for 2 h. The desired mass was detected on LC-MS. The resulting was then quenched to water, extracted with EtOAc, washed with saturated brine, dried over Na₂SO₄ and concentrated. The residue was purified by flash chromatography (eluting with DCM/MeOH from 100/0 to 90/10 in 30 mins) to afford compound (X).

### Preparation of Compound (F-2)

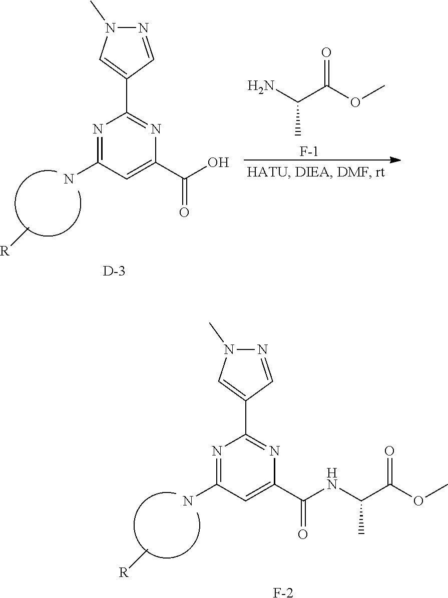

To a solution of compound (D-3) (1.0 eq) in DMF (5 ml) was added HATU (2.0 eq) and DIEA (3.0 eq). After 30 min, compound (F-1) was added. The reaction mixture was stirred at RT for 2 h. The desired mass was detected on LC-MS. The resulting was then quenched to water, extracted with EtOAc, washed with saturated brine, dried over Na₂SO₄ and concentrated. The residue was purified by flash chromatography (eluting with DCM/MeOH from 100/0 to 90/10 in 30 mins) to afford the desired compound (F-2).

### Preparation of Compound (X)

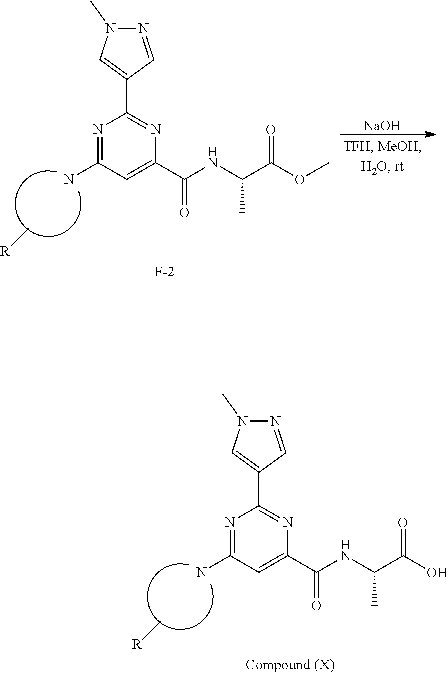

To a solution of compound (F-5) (1.0 eq) in (MeOH:THF:H₂O=6:3:1, 10 mL) was added sodium hydroxide (2.0 eq). The mixture was stirred at RT for 4 h. The desired mass was detected on LC-MS. The reaction solution was concentrated under reduced pressure and adjusted to pH=4 with 2 N HCl. The solution was filtered, and the solids collected to get compound (X).

The compounds in the below table were made in a manner as described above.

| | LCMS | |
|---|---|---|
| | (ESI): | |
| | [M + H]⁺ | Analytics |
| 88 | 397.3 | 1H NMR (400 MHz, DMSO-d6) δ 8.48-8.38 |
| | | (m, 2H), 8.17 (d, J = 9.1 Hz, 1H), 7.45-7.37 |
| | | (m, 1H), 7.32-7.25 (m, 1H), 7.17 (t, J = 8.8 |
| | | Hz, 1H), 6.95 (d, J = 15.2 Hz, 1H), 4.99 (s, |
| | | 2H), 4.94 (d, J = 5.6 Hz, 1H), 4.91 (d, J = 12.7 |
| | | Hz, 2H), 4.10-3.98 (m, 1H), 3.91 (s, 3H), |
| | | 3.53-3.45 (m, 2H), 1.20 (d, J = 6.7 Hz, 3H). |
| 90 | 424.3 | 1H NMR (400 MHz, DMSO-d6) δ 9.11 (d, J = |
| | | 2.5 Hz, 1H), 8.62 (s, 1H), 8.54 (d, J = 8.6 Hz, |
| | | 1H), 8.45 (dd, J = 9.1, 2.5 Hz, 1H), 8.32 (d, |
| | | J = 0.7 Hz, 1H), 8.10 (s, 1H), 6.96 (d, J = 9.1 Hz, |
| | | 1H), 4.92 (t, J = 5.7 Hz, 1H), 4.14-4.03 (m, |
| | | 1H), 3.95 (s, 3H), 3.72 (dd, J = 5.8, 3.7 Hz, |
| | | 4H), 3.63 (dd, J = 5.8, 3.8 Hz, 4H), 3.56-3.48 |
| | | (m, 2H), 1.22 (d, J = 6.8 Hz, 3H). |
| 91 | 500.3 | 1H NMR (400 MHz, DMSO-d6) δ 8.60 (s, |
| | | 1H), 8.55 (d, J = 8.6 Hz, 1H), 8.32 (d, J = 0.6 |
| | | Hz, 1H), 8.27-8.21 (m, 2H), 8.09 (s, 1H), 7.16- |
| | | 7.09 (m, 2H), 4.92 (t, J = 5.6 Hz, 1H), 4.14- |
| | | 4.04 (m, 1H), 3.95 (s, 3H), 3.57-3.48 (m, 2H), |
| | | 3.46 (t, J = 5.7 Hz, 4H), 3.26 (t, J = 5.1 Hz, |
| | | 4H), 2.93 (s, 3H), 1.22 (d, J = 6.7 Hz, 3H). |
| 93 | 397.3 | ¹H NMR (400 MHz, DMSO-d₆) δ 8.47-8.32 |
| | | (m, 2H), 8.14 (s, 1H), 7.49-7.39 (m, 1H), 7.27 |
| | | (d, J = 8.4 Hz, 1H), 7.16 (td, J = 9.0, 2.5 Hz, |
| | | 1H), 6.90 (s, 1H), 4.91 (q, J = 6.9, 6.1 Hz, 3H), |
| | | 4.80 (d, J = 15.1 Hz, 2H), 4.11-3.99 (m, 1H), |
| | | 3.91 (s, 3H), 3.50 (qt, J = 10.9, 5.6 Hz, 2H), |
| | | 1.20 (d, J = 6.7 Hz, 3H). |
| 96 | 437.3 | 1H NMR (400 MHz, DMSO-d6) δ 8.62 (s, 1H), |
| | | 8.57 (d, J = 8.6 Hz, 1H), 8.34 (s, 1H), 8.18- |
| | | 8.10 (m, 3H), 7.13 (d, J = 8.4 Hz, 1H), 4.93 |
| | | (t, J = 5.6 Hz, 1H), 4.16-4.06 (m, 1H), 3.96 |
| | | (s, 3H), 3.80-3.73 (m, 4H), 3.59-3.44 (m, |
| | | 2H), 2.96-2.90 (m, 4H), 2.37 (s, 3H), 1.22 (d, |
| | | J = 6.7 Hz, 3H). |
| 98 | 480.4 | 1H NMR (400 MHz, DMSO-d6) δ 10.21 (s, |
| | | 1H), 8.64 (s, 1H), 8.57 (d, J = 8.6 Hz, 1H), 8.35 |
| | | (t, J = 4.4 Hz, 3H), 8.17 (s, 1H), 7.85 (d, J = |
| | | 8.8 Hz, 2H), 4.91 (t, J = 5.7 Hz, 1H), 4.16- |
| | | 4.05 (m, 1H), 3.96 (s, 3H), 3.70 (s, 4H), 3.58- |
| | | 3.41 (m, 4H), 2.97-2.55 (m, 4H), 1.22 (d, J = |
| | | 6.8 Hz, 3H). |
| 105 | 450.4 | 1H NMR (400 MHz, DMSO-d6) δ 8.67 (s, 1H), |
| | | 8.62 (d, J = 8.6 Hz, 1H), 8.40 (d, J = 8.1 |
| | | Hz, 2H), 8.37 (s, 1H), 8.25 (s, 1H), 7.69 (t, J = |
| | | 7.3 Hz, 2H), 4.93 (s, 1H), 4.16-4.05 (m, 1H), |
| | | 3.66-3.47 (m, 6H), 3.10 (dd, J = 10.2, 4.5 Hz, |
| | | 1H), 2.54 (s, 1H), 2.07-1.90 (m, 1H), 1.72- |
| | | 1.61 (m, 1H), 1.23 (d, J = 6.6 Hz, 3H). |
| 112 | 445.3 | 1H NMR (400 MHz, DMSO-d6) δ 8.66 (s, 1H), |
| | | 8.60 (d, J = 8.6 Hz, 1H), 8.40 (d, J = 8.6 |
| | | Hz, 2H), 8.37 (d, J = 0.7 Hz, 1H), 8.33 (d, J = |
| | | 2.8 Hz, 1H), 8.24 (s, 1H), 7.96 (dd, J = 9.5, 2.8 |
| | | Hz, 1H), 7.81 (s, 1H), 7.80 (d, J = 1.9 Hz, 1H), |
| | | 6.53 (d, J = 9.5 Hz, 1H), 4.92 (t, J = 5.6 Hz, |
| | | 1H), 4.14-4.06 (m, 1H), 3.97 (s, 3H), 3.55 (s, |
| | | 3H), 3.53-3.47 (m, 2H), 1.23 (d, J = 6.7 Hz, |
| | | 3H). |
| 117 | 380.3 | 1H NMR (400 MHz, DMSO-d6) δ 8.51 (d, J = |
| | | 4.9 Hz, 1H), 8.47-8.41 (m, 2H), 8.18 (d, J = |
| | | 9.0 Hz, 1H), 7.87 (s, 1H), 7.40-7.34 (m, 1H), |
| | | 7.00-6.95 (m, 1H), 5.01-4.84 (m, 5H), 4.09- |
| | | 4.01 (m, 1H), 3.92 (s, 3H), 3.67-3.57 (m, |
| | | 1H), 3.18-3.09 (m, 1H), 1.20 (d, J = 6.7 Hz, |
| | | 3H). |
| 125 | 379.3 | 1H NMR (400 MHz, DMSO-d6) δ 8.45-8.40 |
| | | (m, 2H), 8.16 (s, 1H), 7.44 (d, J = 5.5 Hz, 2H), |
| | | 7.35 (dd, J = 5.6, 3.1 Hz, 2H), 6.95 (s, 1H), |
| | | 4.96 (s, 2H), 4.92 (d, J = 5.5 Hz, 1H), 4.86 (s, |
| | | 2H), 4.09-4.00 (m, 1H), 3.92 (s, 3H), 3.53- |
| | | 3.46 (m, 2H), 1.20 (d, J = 6.7 Hz, 3H). |
| 126 | 393.3 | ¹H NMR (400 MHz, DMSO-d₆) δ 8.90 (d, J = |
| | | 7.9 Hz, 1H), 8.44 (s, 1H), 8.19 (s, 1H), 7.44 (q, |
| | | J = 4.2 Hz, 2H), 7.34 (dd, J = 5.6, 3.1 Hz, 2H), |
| | | 6.94 (s, 1H), 4.96 (s, 2H), 4.86 (s, 2H), 4.56- |
| | | 4.45 (m, 1H), 3.92 (s, 3H), 1.48 (d, J = 7.3 Hz, |
| | | 3H). |
| 127 | 482.3 | 1H NMR (400 MHz, DMSO-d6) δ 8.61 (d, J = |
| | | 8.3 Hz, 1H), 8.40 (s, 1H), 8.20 (s, 1H), 7.46- |
| | | 7.28 (m, 5H), 6.91 (s, 1H), 4.93 (s, 2H), 4.81 |
| | | (s, 2H), 4.16-4.05 (m, 1H), 3.90 (s, 3H), 3.22 |
| | | (t, J = 6.2 Hz, 2H), 2.66-2.57 (m, 1H), 1.25 |
| | | (d, J = 6.6 Hz, 3H), 0.93 (dd, J = 6.6, 2.5 Hz, |
| | | 4H). |

### Synthesis

### Biological Assay

The AhR reporter assay was performed following the protocol of the Human Aryl Hydrocarbon Receptor (AhR) Reporter Assay System (INDIGO Bioscience, #IB06001-32). AhR Reporter Cell was thawed and pre-incubated in Cell Recovery Medium (CRM) for 6 hours. The media was then removed and the cells were incubated with Compound Screening Medium (CSM) supplemented with 200 μM kynurenic acid for 24 hours in the absence (negative control) or presence of 10 nM test compounds. For positive inhibition control, cells activated with 200 μM kynurenic acid were incubated in the presence of literature AHR antagonists; alternatively un-activated cells were used as positive inhibition control. Following 24 hours incubation, the media was removed and Luciferase Detection Reagent (LDR) added. The firefly luciferase activity was quantified using a plate reader. Results were normalized by positive and negative controls.

The results of AHR inhibition at 10 nM for compounds as disclosed herein is reported in the below table.

| | Inhibition |
|---|---|
| | @10 nM |
| 1 | 56% |
| 2 | 51% |
| 3 | 66% |
| 4 | 53% |
| 5 | 40% |
| 6 | 96% |
| 7 | 90% |
| 8 | 83% |
| 9 | 29% |
| 10 | 28% |
| 11 | 3% |
| 12 | 72% |
| 13 | 64% |
| 14 | 85% |
| 15 | 99% |
| 16 | 82% |
| 17 | 35% |
| 20 | 70% |
| 21 | −0.3% |
| 25 | 8% |
| 26 | 24% |
| 27 | 45% |
| 28 | −5% |
| 29 | 8% |
| 30 | −5% |
| 31 | 24% |
| 32 | −16% |
| 34 | 6% |
| 35 | 5% |
| 39 | −4% |
| 41 | 39% |
| 42 | 16% |
| 44 | 44% |
| 52 | 35% |
| 54 | 1% |
| 56 | 99% |
| 57 | 29% |
| 59 | 24% |
| 60 | 21% |
| 61 | 62% |
| 62 | 29% |
| 63 | 58% |
| 64 | 49% |
| 65 | 7% |
| 66 | 69% |
| 67 | 33% |
| 68 | 62% |
| 69 | 60% |
| 70 | 87% |
| 71 | 37% |
| 72 | 13% |
| 73 | 23% |
| 74 | 13% |
| 75 | 24% |
| 76 | 1% |
| 77 | 4% |
| 88 | 21% |
| 90 | 1% |
| 91 | 7% |
| 93 | 99% |
| 96 | 10% |
| 98 | 5% |
| 105 | −3% |
| 112 | 10% |
| 117 | 46% |
| 119 | 2% |
| 120 | −10% |
| 121 | 4% |
| 122 | 46% |
| 123 | 91% |
| 124 | 77% |
| 125 | 23% |
| 126 | 5% |
| 127 | 52% |
| 128 | 4% |
| 129 | 7% |

1. A compound, or pharmaceutically acceptable salt thereof, having a structure of Formula (I) or Formula (II): 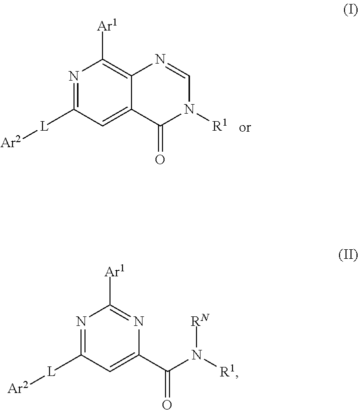
L is a bond, C(O), C₂₋₃alkenylene, or C₂₋₃alkynylene;
Ar¹ is phenyl, C₃₋₈cycloalkyl, or 4-8 membered aromatic or non-aromatic heterocyclyl having 1-3 ring heteroatoms selected from O, N, and S;
each R^{A} is independently halo, OH, ═O, CN, C₁₋₆alkyl, C₁₋₆haloalkyl, C₁₋₆hydroxyalkyl, C₁₋₆alkoxy, C₁₋₆haloalkoxy, cyclopropyl, NR^{N}R^{N}, C(O)NR^{N}R^{N}, CO₂R^{N}, C(O)R^{N}, or SO₂—C₁₋₆alkyl;
each R^{N} is independently H or C₁₋₆alkyl;
Ar² is 5-10 membered aromatic or non-aromatic ring having 0-3 ring heteroatoms selected from O, N, and S;
each R^{B} is independently halo, CN, C₁₋₆alkyl, OH, ═O, C₁₋₆hydroxyalkyl, C(O)R^{N}, CO₂R^{N}, C(O)NR^{N}R^{N}, Het, C(O)—C₀₋₂alkylene-Het, or N(R^{N})—C₀₋₃alkylene-C(O)—C₀₋₃alkylene-Het;
Het is a 4-8-membered aromatic or non-aromatic ring having 0-3 ring heteroatoms selected from N, O, and S, and is optionally substituted with 1, 2, or 3 R^{C};
each R^{C} is independently halo, OH, ═O, CN, C₁₋₆alkyl, C₁₋₆haloalkyl, C₁₋₆hydroxyalkyl, C₁₋₆alkoxy, C₁₋₆haloalkoxy, NR^{N}R^{N}, C(O)NR^{N}R^{N}, CO₂R^{N}, C(O)R^{N}, or SO₂—C₁₋₆alkyl;
R¹ is C₁₋₆alkyl, C₁₋₆alkylene-R^{D}, C₁₋₆alkylene-C(O)R^{D}, C₁₋₆alkylene-NHSO₂cyclopropyl, or C₁₋₆alkylene-NR^{N}—C(O)R^{N}; and
R^{D} is OR^{N} or NR^{N}R^{N},
with the proviso that
(i) for compounds of Formula (I), when L is a bond, then (a) Ar² is substituted with at least one R^{B} selected from Het, C(O)—C₀₋₂alkylene-Het, and N(R^{N})—C₀₋₃alkylene-C(O)—C₀₋₃alkylene-Het or (b) Ar² comprises a 8-10-membered bicyclic aromatic or non-aromatic ring having 1-3 ring heteroatoms and is substituted with 0-3 R^{B}; and
(ii) for compounds of Formula (II), when L is a bond, Ar² is phenyl or a 6-membered aromatic heterocyclyl, Ar² is substituted with 1 R^{B}, and R^{B} is Het, then (a) Het is attached to Ar² via a carbon ring atom of Het or (b) Het is substituted with 1, 2, or 3 R^{C}.

2. (canceled)

3. The compound or salt of claim 1, wherein R¹ is C₁₋₆alkyl or C₁₋₆alkylene-R^{D}.

4.-6. (canceled)

7. The compound or salt of claim 1, having a structure of Formula (Ia) or Formula (IIa):

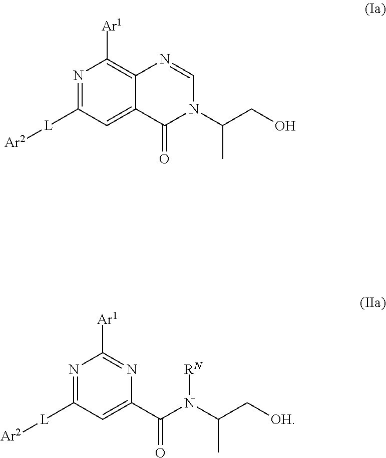

8. (canceled)

9. The compound or salt of claim 1, wherein Ar¹ is heterocyclyl comprising 4-8 total ring atoms, wherein 1-3 of the ring atoms are selected from O, N, and S.

10. The compound or salt of claim 1, wherein Ar¹ is unsubstituted, or substituted with 1 or 2 R^{A}; wherein at least one R^{A} is C₁₋₆alkyl, C₁₋₆haloalkyl, or C₁₋₆alkoxy, or at least one R^{A} is methyl.

11.-12. (canceled)

13. The compound or salt of claim 1, having a structure of Formula (Ib) or Formula (IIb):

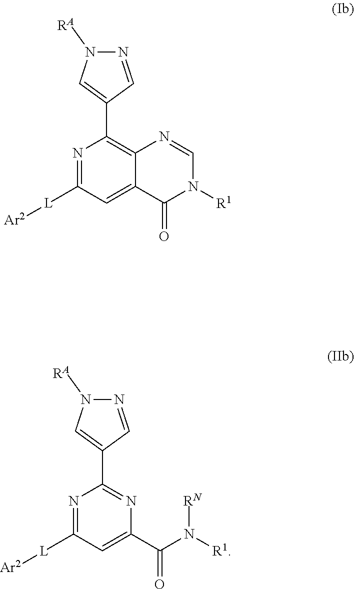

14.-15. (canceled)

16. The compound or salt of claim 1, having a structure of Formula (Ic) or Formula (IIc):

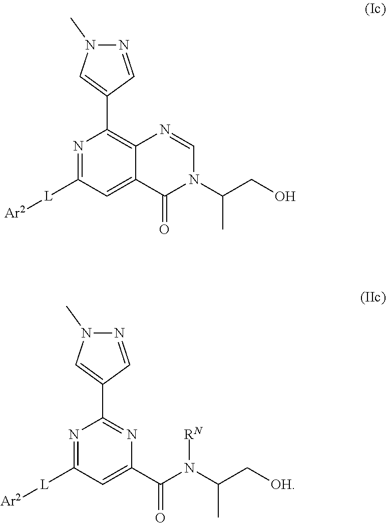

17. (canceled)

18. The compound or salt of claim 1, wherein L is a bond, C₂₋₃alkenylene, or C₂₋₃alkynylene.

19.-21. (canceled)

22. The compound or salt of claim 1, wherein Ar² is a 5-10 membered aromatic or non-aromatic ring having 1-3 ring heteroatoms selected from O, N, and S; or an 8-10 bicyclic aromatic or non-aromatic ring having 1-3 ring heteroatoms selected from O, N, and S.

23.-24. (canceled)

25. The compound or salt of claim 1, wherein Ar² is unsubstituted or substituted with 1, 2, or 3 R^{B}, wherein at least one R^{B} is halo, CN, C₁₋₆alkyl, OH, ═O, C₁₋₆hydroxyalkyl, C(O)R^{N}, CO₂R^{N}, C(O)NR^{N}R^{N}, Het, C(O)—C₀₋₂alkylene-Het, or N(R^{N})—C₀₋₃alkylene-C(O)—C₀₋₃alkylene-Het, and wherein Het is unsubstituted or substituted with 1 or 2 R^{C}, at least one R^{C} is halo, OH, ═O, C₁₋₆alkyl, CO₂R^{N}, NR^{N}R^{N}, or SO₂—C₁₋₆alkyl.

26.-33. (canceled)

34. The compound or salt of claim 25, wherein Het is

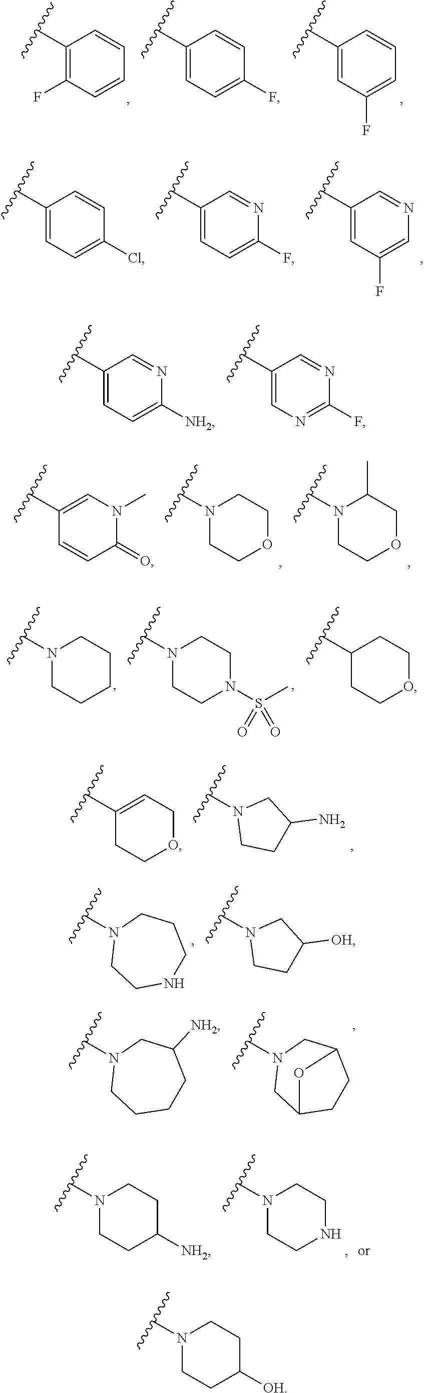

35. The compound or salt of claim 1, wherein Ar² is

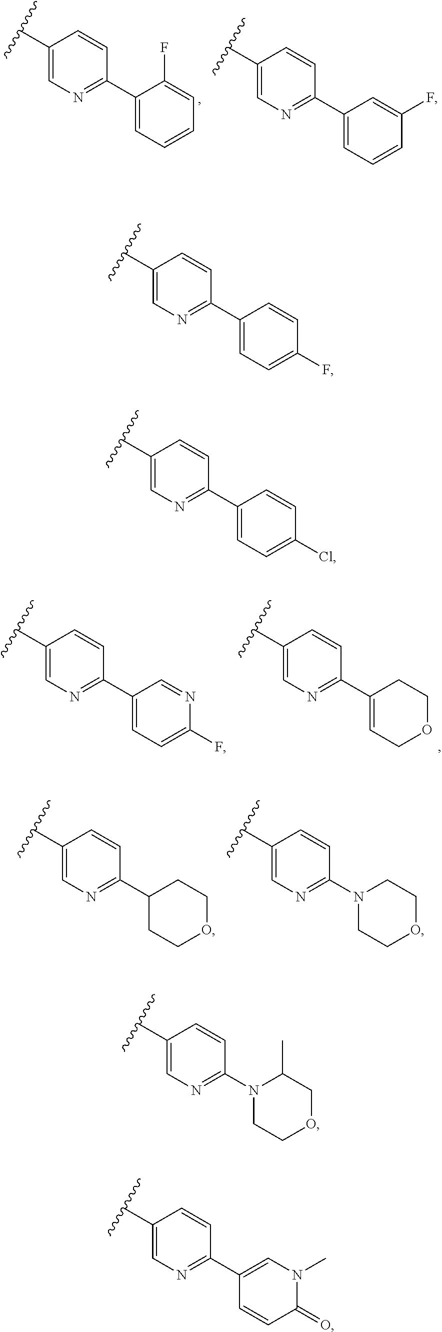

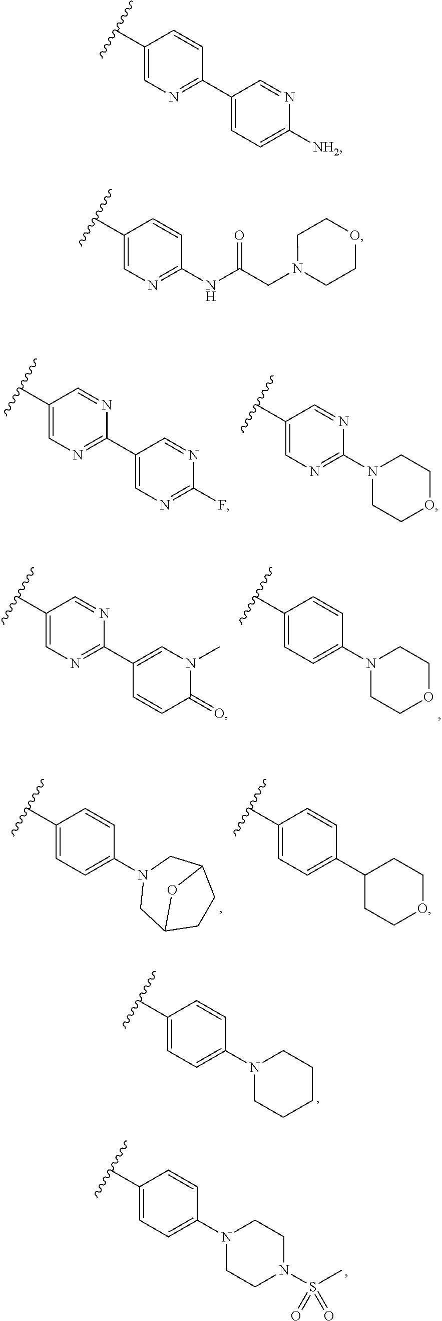

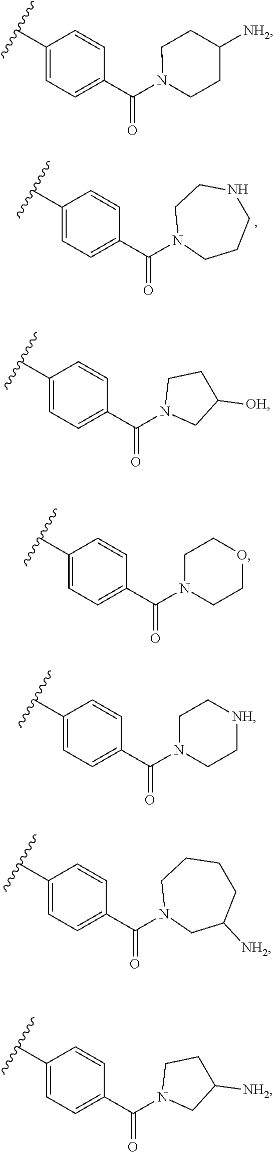

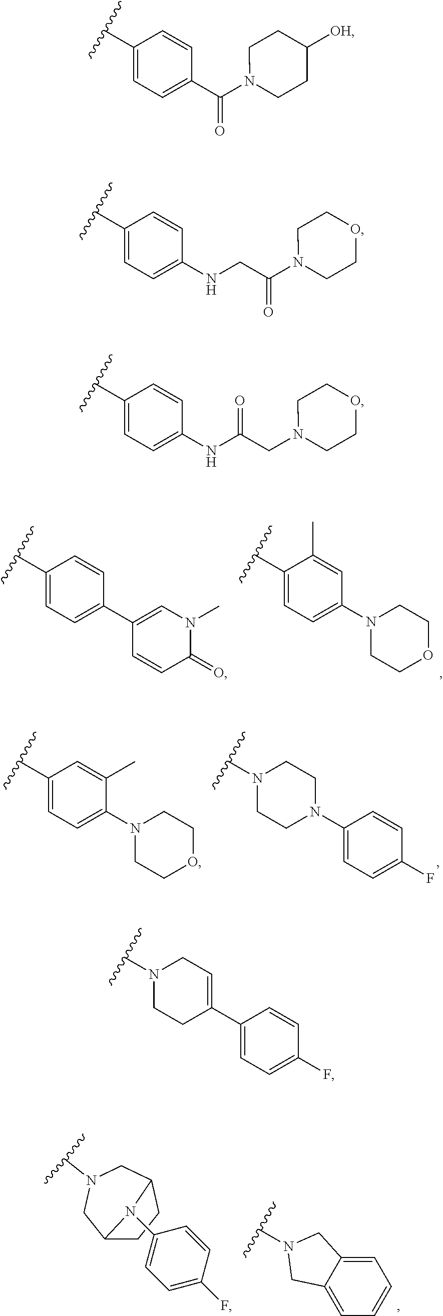

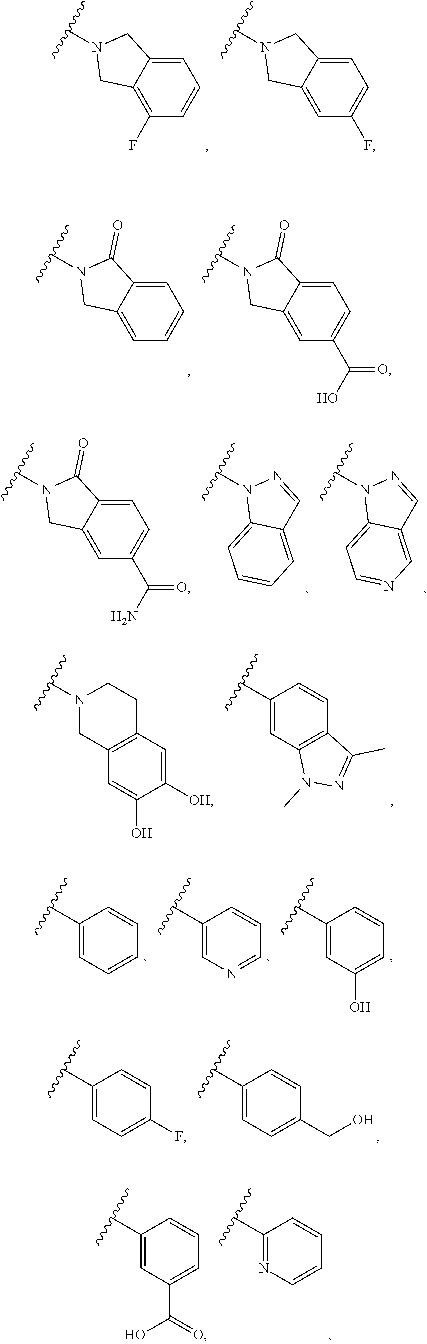

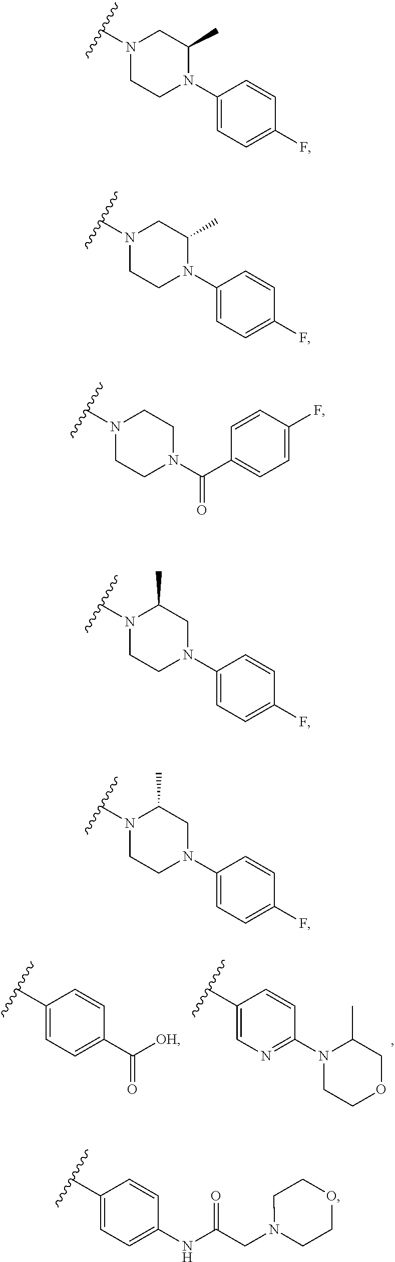

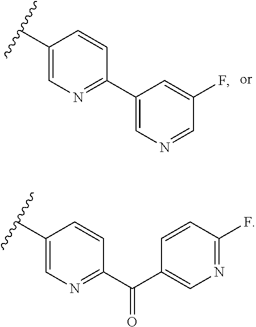 [SMILES: *c1ccc(-c2cncc(F)c2)nc1 ; "or" ; *c1ccc(C(=O)c2ccc(F)nc2)nc1]

36. (canceled)

37. A compound as listed in Table 1, or a pharmaceutically acceptable salt thereof.

38. A pharmaceutical formulation comprising the compound or salt of claim 1 and a pharmaceutically acceptable excipient.

39. A method of treating or preventing a disease or disorder associated with aberrant aryl hydrocarbon receptor (AHR) activity in a subject, comprising administering to the subject a therapeutically effective amount of the compound or salt of claim 1.

40. The method of claim 39, wherein the disease or disorder is an inflammatory disease or disorder, or a disease or disorder of the gastrointestinal tract, skin, lung, central nervous system, pancreas, eye, bones bone joints, a neuroinflammatory disease, or a neurodegenerative disease; or is diabetes, cancer, a viral infection, or a bacterial infection.

41.-49. (canceled)

50. The method of claim 39, further comprising administering to the patient a therapeutic agent.

51. The method of claim 50, wherein the therapeutic agent is an anti-inflammatory agent or an immune checkpoint inhibitor.

52.-53. (canceled)

54. A compound or salt of claim 1 for use in the treatment or prevention of a disease or disorder associated with aberrant aryl hydrocarbon receptor (AHR) activity.

55. Use of the compound or salt of claim 1 for treating or preventing a disease or disorder associated with aberrant aryl hydrocarbon receptor (AHR) activity.
